(19) United States
(12) Patent Application Publication
(10) Pub. No.: US 2021/0347866 A1
(43) Pub. Date: Nov. 11, 2021

(54) PEPTIDE IMMUNOGEN CONSTRUCTS DIRECTED AGAINST DIPEPTIDE REPEAT PROTEINS FROM C9ORF72

(71) Applicants: UNITED NEUROSCIENCE, Grand Cayman (KY); UNS IP HOLDINGS, LLC, Dallas, TX (US)

(72) Inventors: Chang Yi WANG, Cold Spring Harbor, NY (US); Ajay VERMA, Needham, MA (US)

(21) Appl. No.: 17/282,336

(22) PCT Filed: Oct. 1, 2019
(86) PCT No.: PCT/US2019/053967

§ 371 Date: Apr. 1, 2021

Related U.S. Application Data
(60) Provisional application No. 62/739,794, filed on Oct. 1, 2018.

(51) Int. Cl. C07K 16/18; C07K 14/47; A61K 47/54; A61K 39/39

(52) U.S. Cl. CPC C07K 16/18; C07K 14/47; A61K 2039/55561; A61K 39/39; A61K 47/549

(57) ABSTRACT

The present disclosure is directed to dipeptide repeat (DPR) peptide immunogen constructs, compositions containing the constructs, antibodies elicited by the constructs, and methods for making and using the constructs and compositions thereof. The disclosed DPR peptide immunogen constructs contain a B cell epitope derived from a DPR protein from C9orf72, including repeats of poly-GA, poly-GP, poly-GR, poly-PR, and poly-PA, linked to a heterologous T helper cell (Th) epitope directly or through an optional heterologous spacer. The B cell epitope portion of the peptide immunogen constructs contain about 10 to about 25 repeats of the respective dipeptide sequence. The disclosed peptide immunogen constructs stimulate the generation of highly specific antibodies directed against the DPR sequences. The disclosed peptide immunogen constructs can be used as an immunotherapy for patients suffering from amyotrophic lateral sclerosis (ALS), frontotemporal dementia (FTD), and/or any other condition caused by the presence of DPRs.

The present application is a PCT International Application that claims the benefit of U.S. Provisional Application Ser. No. 62/739,794, filed Oct. 1, 2018, which is incorporated herein by reference in its entirety.

### FIELD OF THE INVENTION

This disclosure relates to peptide immunogen constructs based on portions of a Dipeptide Repeat (DPR) protein from C9orf72 and formulations thereof for the prevention and treatment of amyotrophic lateral sclerosis (ALS) and frontotemporal dementia (FTD).

### BACKGROUND OF THE INVENTION

Expansion of a GGGGCC hexanucleotide sequence within the intron of the human C9ORF72 gene is associated with both amyotrophic lateral sclerosis (ALS) and frontotemporal dementia (FTD) in humans (WO 2014/159247 by Ranum, et al.). It has been shown that the unconventional non-ATG translation of the sense transcript in the three alternate reading frames, i.e., of the expanded hexanucleotide repeats, results in the production, generation, and aggregation of three different polypeptides, each composed of repeating units of two amino acids (dipeptide repeats, DPRs), i.e., poly-(Gly-Ala; GA), poly-(Gly-Pro; GP) and poly-(Gly-Arg; GR) (WO 2016/050822A2 by Montrasio). Furthermore, translation of corresponding antisense transcripts results in the generation of poly-(Pro-Arg; PR), poly-(Pro-Ala; PA), and poly-(Gly-Pro; GP). These C9ORF72-dipeptide repeat (DPR) expansions have been shown to account for up to 30% of FTD, 50% of ALS, and 80% of FTD-ALS patients with the highest mutation frequencies observed in US and EU Caucasian populations.

ALS is a debilitating disease with varied etiology that affects 2 in 100,000 people (WO 2014/159247 by Ranum, et al.; U.S. Pat. No. 9,448,232 by Petrucelli). ALS has traditionally been considered a disorder in which degeneration of upper and lower motor neurons and is characterized by rapidly progressing weakness, muscle atrophy, muscle spasticity, difficulty speaking (dysarthria), difficulty swallowing (dysphagia), and difficulty breathing (dyspnea). Although the order and rate of symptoms varies from person to person, eventually most subjects are not able to walk, get out of bed on their own, or use their hands and arms and most subjects with ALS will eventually die from respiratory failure, usually within three to five years from the onset of symptoms. ALS is increasingly recognized to be a multisystem disorder with impairment of frontotemporal functions such as cognition and behavior in up to 50% of patients. Riluzole (RILUTEK®) is the only currently available treatment for ALS and only slows progression and increases survival to a modest extent.

FTD belongs to a group of clinically, pathologically and genetically heterogeneous disorders associated with atrophy or shrinkage in the frontal lobe and temporal lobe of the brain. It is the second most common cause of early-onset dementia, behind Alzheimer's disease. Cognitive symptoms are variable and include dementia, changes of the behavior, as well as personality, language dysfunctions, and/or psychosis that are due to the degeneration of the frontal and temporal cortex. Due to its symptoms FTD can be divided into three groups (i) behavioral-variant frontotemporal dementia (bvFTD), which involves changes in personality, behavior, and judgment; (ii) primary progressive aphasia (PPA), which involves changes in the ability to communicate—to use language to speak, read, write, and understand what others are saying; and (iii) corticobasal syndrome (CBS) and progressive supranuclear palsy (PSP), which affect the control of movement, thinking, and language abilities. Patients with FTD die 5-10 years after symptom onset, since no suitable therapy is available. However, 50% of FTD patient were shown to have a positive family history and, compared to ALS, FTD seems to represent a disease continuum with a shared underlying pathogenesis.

New methods for diagnosing and treating ALS and/or FTD would greatly benefit ALS and FTD subjects. The genetic mutation leading to RNA expansion in the C9orf72 leads to an autosomal dominant form of hereditary neurodegeneration characterized by the presence of both ALS and FTD. The C9orf72 for of ALS/FTD is no recognized as the most common genetic form of these diseases.

### SUMMARY OF THE INVENTION

The present disclosure is directed to individual peptide immunogen constructs targeting portions of a Dipeptide Repeat (DPR) protein from C9orf72 and formulations thereof for the prevention and treatment of amyotrophic lateral sclerosis (ALS) and frontotemporal dementia (FTD). The present disclosure is also directed to compositions containing the peptide immunogen constructs, methods of making and using the peptide immunogen constructs, and antibodies produced by the peptide immunogen constructs.

In certain embodiments, the DPR peptide immunogen construct can be represented by the formula:

{(Th)ₘ-(A)ₙ-(DPR)-(A)ₙ-(Th)ₘ}_{y}-X

wherein

Th is a heterologous T helper epitope;

A is a heterologous spacer;

(DPR) is a B cell epitope having repeats of poly-GA, poly-GP, poly-GR, poly-PR, or poly-PA;

X is an α-COOH or α-CONH₂ of an amino acid;

each m is from 0 to about 4;

each n is from 0 to about 10; and

y is from 1 to about 5.

The various components of the disclosed DPR peptide immunogen construct are described below.

### REFERENCES

1. RANUM, L., et al. “Di-amino acid repeat-containing proteins associated with ALS”, WO 2014/159247, Oct. 2, 2014
2. PETRUCELLI, L., et al. “Methods and materials for detecting C9ORF72 hexanucleotide repeat expansion positive frontotemporal lobar degeneration or C9ORF72 hexanucleotide repeat expansion positive amyotrophic lateral sclerosis”, U.S. Pat. No. 9,448,232, Sep. 20, 2016
3. MONTRASIO, F., et al. “Human-derived anti-dipeptide repeats (DPRs) antibody”, WO 2016/050822, Apr. 7, 2016
4. FREIBAUM, B. D., et al., “The role of dipeptide repeats in C9ORF72-related ALS-FTD”, Frontiers in Molecular Neuroscience, 10(35): 1-35 (2017)

### BRIEF DESCRIPTION OF THE DRAWINGS

FIG. 1—is a schematic of translation products of the three alternate frames of the sense RNA transcript generated from the expanded C9ORF72 region GGGGCC repeat (SEQ ID NO: 225) that would generate repeating dipeptides of (Glycine-Arginine; GR)ₙ (SEQ ID NO: 226), (Glycine-Proline; GP)ₙ (SEQ ID NO: 227), and (Glycine-Alanine; GA)ₙ (SEQ ID NO: 228), and the translation products of the three alternate frames of the anti-sense RNA GGCCCC repeat (SEQ ID NO: 229) that would generate repeating dipeptides of (Glycine-Proline; GP)ₙ (SEQ ID NO: 230), (Proline-Arginine; PR)ₙ (SEQ ID NO: 231), and (Proline-Alanine; PA)ₙ (SEQ ID NO: 232).

FIG. 2—is a table that summarizes several structural and functional characteristics of individual DPR species and their influence on their interactions and toxicity.

FIGS. 3A-3I—are graphs illustrating the antibody titers obtained after immunizing guinea pigs with the peptide immunogen constructs disclosed herein. Specifically, the antibody titers obtained after immunization with poly-GA constructs (SEQ ID NOs: 68, 69, 70, and 88) are shown in FIGS. 3A-3D, respectively. Antibody titers obtained after immunization with poly-GP constructs (SEQ ID NOs: 98 and 99) are shown in FIGS. 3E-3F, respectively. Antibody titers obtained after immunization with poly-GR constructs (SEQ ID NOs: 130 and 148) are shown in FIGS. 3G-3H, respectively. Antibody titers obtained after immunization with a poly-GR construct (SEQ ID NO: 161) is shown in FIG. 3I.

FIGS. 4A-4D—are graphs illustrating the antibody titers obtained after immunizing guinea pigs with the peptide immunogen constructs disclosed herein. Specifically, the antibody titers obtained after immunization with poly-GA constructs (SEQ ID NOs: 68, 69, 70, 80, and 88) are shown with high titers; whereas antibody titers obtained after immunization with (GA)₅ or (GA)₁₀ and (GA)₁₅ (SEQ ID NOs: 1 and 2), containing the poly-GA sequences that are not linked to UBITh® peptides for immunogenicity enhancement, were not immunogenic, as shown in FIG. 4A. The antibody titers obtained after immunization with poly-GP constructs (SEQ ID NOs: 98, 99, and 110) are shown with high titers; whereas antibody titers obtained after immunization with (GP)₁₀ and (GP)₁₅ (SEQ ID NOs: 4 and 5), containing the poly-GP sequences that are not linked to UBITh® peptides for immunogenicity enhancement, were not immunogenic, as shown in FIG. 4B. The antibody titers obtained after immunization with poly-GR constructs (SEQ ID NOs: 130 and 148) are shown with high titers; whereas antibody titers obtained after immunization with (GR)₁₀, (GR)₁₅, and (GR)₂₅ (SEQ ID NOs: 7, 8, and 9), containing poly-GR sequences that are not linked to UBITh® peptides for immunogenicity enhancement, were not immunogenic, as shown in FIG. 4C. The antibody titers obtained after immunization with poly-PR constructs (SEQ ID NOs: 161 and 173) are shown with high titers; whereas antibody titers obtained after immunization with (PR)₁₀ (SEQ ID NO: 10), containing a poly-PR sequence that is not linked to UBITh® peptides for immunogenicity enhancement, displayed immunogenicity slightly above background, as shown in FIG. 4D.

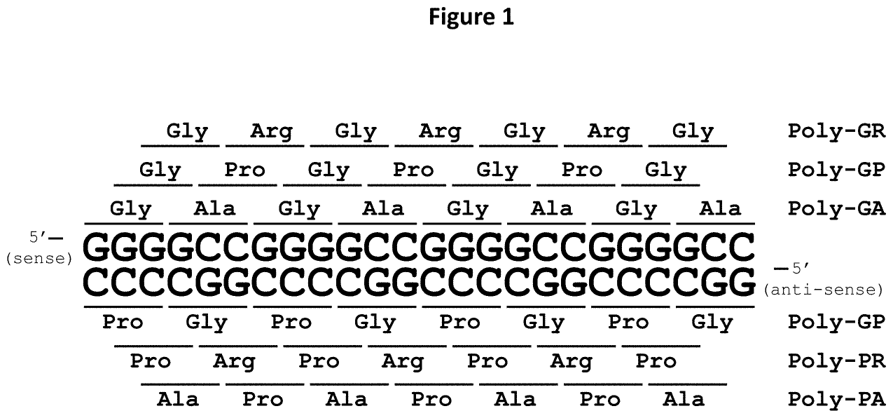

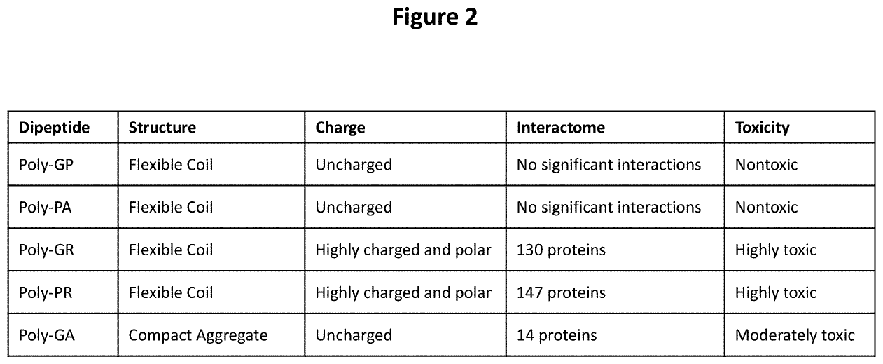

### DETAILED DESCRIPTION OF THE INVENTION

The present disclosure is directed to individual peptide immunogen constructs targeting portions of a Dipeptide Repeat (DPR) protein from C9orf72 and formulations thereof for the prevention and treatment of amyotrophic lateral sclerosis (ALS) and frontotemporal dementia (FTD). The present disclosure is also directed to compositions containing the peptide immunogen constructs, methods of making and using the peptide immunogen constructs, and antibodies produced by the peptide immunogen constructs.

The disclosed peptide immunogen constructs contain a B cell epitope and a T helper cell epitope and have about 20 or more amino acids.

The peptide immunogen constructs contain a B cell epitope from portions of a Dipeptide Repeat (DPR) protein from C9orf72, including sequences of poly-GA repeats, poly-GP repeats, poly-GR repeats, poly-PR repeats, and poly-PA repeats. The B cell epitope can be linked to a heterologous T helper cell (Th) epitope derived from pathogen proteins through an optional heterologous spacer. The disclosed peptide immunogen constructs stimulate the generation of highly specific antibodies directed against the DPRs. The disclosed peptide immunogen constructs can be used as an immunotherapy for patients suffering from amyotrophic lateral sclerosis (ALS), frontotemporal dementia (FTD), and/or any other condition caused by the presence of DPRs.

The B cell epitope portion of the peptide immunogen constructs have amino acid sequences derived from a Dipeptide Repeat (DPR) Protein from C9orf72, including sequences of poly-GA repeats (SEQ ID NOs: 1-3), poly-GP repeats (SEQ ID NOs: 4-5), poly-GR repeats (SEQ ID NOs: 7-9), poly-PR repeats (SEQ ID NOs: 10-12), and poly-PA repeats (SEQ ID NOs: 13-15), as shown in Table 1.

The peptide immunogen constructs of the present disclosure contain a heterologous Th epitope amino acid sequence derived from a pathogenic protein (e.g., SEQ ID NOs: 16-67), as shown in Table 2. In certain embodiments, the heterologous Th epitope is derived from natural pathogens, such as Diphtheria Toxin (SEQ ID NO: 55), Plasmodium Falciparum (SEQ ID NO: 66), Cholera Toxin (SEQ ID NO: 48). In other embodiments, the heterologous Th epitope is an idealized artificial Th epitope derived from Measles Virus Fusion protein (MVF 1 to 5) or Hepatitis B Surface Antigen (HBsAg 1 to 3) in the form of either single sequence (e.g., SEQ ID NOs: 21, 22, 32, 33, and 43-46) or combinatorial sequences (e.g., SEQ ID NOs: 20, 25, 28, 31, 39, and 42). The peptide immunogen constructs of the present disclosure are capable of eliciting antibody production directed against the B cell epitope region of the constructs without activating an inflammatory T cell response.

In some embodiments, the peptide immunogen constructs contain a B cell epitope from a DPR linked to a heterologous T helper cell (Th) epitope through an optional heterologous spacer. In certain embodiments, the peptide immunogen constructs contain a B cell antigenic site having the amino acid sequence from a DPR (e.g., SEQ ID NOs: 1 to 15) linked to a heterologous Th epitope derived from a pathogenic protein (e.g., SEQ ID NOs: 16 to 67) through an optional heterologous spacer. In some embodiments, the optional heterologous spacer is a molecule or chemical structure capable of linking two amino acids and/or peptides together. In certain embodiments, the spacer is a naturally occurring amino acid, a non-naturally occurring amino acid, or a combination thereof. In specific embodiments, the peptide immunogen constructs have the amino acid sequence of SEQ ID NOs: 68 to 217, shown in Tables 3-7.

The present disclosure is also directed to compositions containing a DPR peptide immunogen construct. In some embodiments, the disclosed compositions contain more than one DPR peptide immunogen construct. In certain embodiments, the compositions contain a mixture of DPR peptide immunogen constructs (e.g., any combination of SEQ ID NOs: 68 to 217) to cover a broad genetic background in patients. Compositions containing a mixture of peptide immunogen constructs can lead to a higher percentage in responder rate upon immunization for the prevention and/or treatment of ALS, FTD, and/or any other condition caused by the presence of DPRs compared to compositions containing only a single peptide immunogen construct.

The present disclosure is also directed to pharmaceutical compositions for the prevention and/or treatment of ALS, FTD, or any other condition caused by the presence of DPRs. In some embodiments, the pharmaceutical compositions contain the disclosed peptide immunogen constructs in the form of a stabilized immunostimulatory complex formed through electrostatic associations by mixing a CpG oligomer with a composition containing a peptide immunogen complex. Such stabilized immunostimulatory complexes are able to further enhance the immunogenicity of the peptide immunogen constructs. In some embodiments, the pharmaceutical compositions contain adjuvants such as mineral salts, including alum gel (ALHYDROGEL), aluminum phosphate (ADJUPHOS), or water-in-oil emulsions including MONTANIDE ISA 51 or 720.

The present disclosure is also directed to antibodies directed against the disclosed DPR peptide immunogen constructs. In particular, the peptide immunogen constructs of the present disclosure are able to stimulate the generation of highly specific antibodies that are cross-reactive with the DPR amino acid sequences (SEQ ID NOs: 1-15) when administered to a subject. The highly specific antibodies produced by the peptide immunogen constructs are cross reactive with recombinant DPR-containing proteins. The disclosed antibodies bind with high specificity to the respective DPR without much, if any, directed to the heterologous Th epitopes employed for immunogenicity enhancement, which is in sharp contrast to the conventional protein or other biological carriers used for such peptide antigenicity enhancement.

The present disclosure also includes methods for preventing and/or treating ALS, FTD, and/or any other condition caused by the presence of DPRs using the disclosed peptide immunogen constructs and/or antibodies directed against the peptide immunogen constructs. In some embodiments, the methods for preventing and/or treating ALS, FTD, and/or any other condition caused by the presence of DPRs including administering to a host a composition containing a disclosed peptide immunogen construct. In certain embodiments, the compositions utilized in the methods contain a disclosed peptide immunogen construct in the form of a stable immunostimulatory complex with negatively charged oligonucleotides, such as CpG oligomers, through electrostatic association, which complexes are further supplemented, optionally, with mineral salts or oil as adjuvant, for administration to patients with ALS, FTD, and/or any other condition caused by the presence of DPRs. The disclosed methods also include dosing regimens, dosage forms, and routes for administering the peptide immunogen constructs to a host at risk for, or with, ALS, FTD, and/or any other condition caused by the presence of DPRs.

The section headings used herein are for organizational purposes only and are not to be construed as limiting the subject matter described. All references or portions of references cited in this application are expressly incorporated by reference herein in their entirety for any purpose.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all amino acid sizes, and all molecular weight or molecular mass values, given for polypeptides are approximate, and are provided for description. Although methods and materials similar or equivalent to those described herein can be used in the practice or testing of the disclosed method, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Dipeptide Repeat (DPR) Peptide Immunogen Construct

The present disclosure provides peptide immunogen constructs containing a B cell epitope with an amino acid sequence from a DPR covalently linked to a heterologous T helper cell (Th) epitope directly or through an optional heterologous spacer.

The phrase “DPR peptide immunogen construct” or “peptide immunogen construct”, as used herein, refers to a peptide containing (a) a B cell epitope having about 20 or more amino acid residues of a DPR; (b) a heterologous Th epitope; and (c) an optional heterologous spacer.

In certain embodiments, the DPR peptide immunogen construct can be represented by the formula:

{(Th)ₘ-(A)ₙ-(DPR)-(A)ₙ-(Th)ₘ}_{y}-X

wherein

Th is a heterologous T helper epitope;

A is a heterologous spacer;

(DPR) is a B cell epitope having repeats of poly-GA, poly-GP, poly-GR, poly-PR, or poly-PA;

X is an α-COOH or α-CONH₂ of an amino acid;

each m is from 0 to about 4;

each n is from 0 to about 10; and

y is from 1 to about 5.

The various components of the disclosed DPR peptide immunogen construct are described below.

a. B Cell Epitopes (DPRs)

The B cell epitope portion of the peptide immunogen constructs have amino acid sequences derived from a Dipeptide Repeat (DPR) protein from C9orf72, including repeats of poly-GA, poly-GP, poly-GR, poly-PR, or poly-PA.

In some embodiments, the B cell epitope is a DPR having more than one repeat of poly-GA, poly-GP, poly-GR, poly-PR, or poly-PA. The B cell epitope can have between 2 to 50 repeats of poly-GA, poly-GP, poly-GR, poly-PR, or poly-PA. For example, the B cell epitope can have 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 repeats of poly-GA, poly-GP, poly-GR, poly-PR, or poly-PA.

In some embodiments, the B cell epitope is a DPR having about 10 to about 25 repeats of poly-GA, poly-GP, poly-GR, poly-PR, or poly-PA. In certain embodiments, the DPR contains 10, 15, or 25 repeats of poly-GA (SEQ ID NOs: 1-3), poly-GP (SEQ ID NOs: 4-5), poly-GR (SEQ ID NOs: 7-9), poly-PR (SEQ ID NOs: 10-12), or poly-PA (SEQ ID NOs: 13-15), as shown in Table 1.

In some embodiments, the B cell epitope portion of the DPR peptide immunogen constructs does not contain an endogenous T helper cell epitope. Accordingly, the B cell epitope portion is not immunogenic, or has very little immunogenicity, on its own.

b. Heterologous T Helper Cell Epitopes (Th Epitopes)

The present disclosure provides peptide immunogen constructs containing a B cell epitope derived from a Dipeptide Repeat (DPR) protein from C9orf72 covalently linked to a heterologous T helper cell (Th) epitope directly or through an optional heterologous spacer.

The heterologous Th epitope in the peptide immunogen construct enhances the immunogenicity of the DPR fragments, which facilitates the production of specific high titer antibodies directed against the DPR through rational design.

The term “heterologous”, as used herein, refers to an amino acid sequence that is derived from an amino acid sequence that is not part of, or homologous with, the wild-type sequence of the DPR. Thus, a heterologous Th epitope is a Th epitope derived from an amino acid sequence that is not naturally found in the DPR (i.e., the Th epitope is not autologous to the DPR). Since the Th epitope is heterologous to the DPR, the natural amino acid sequence of the DPR is not extended in either the N-terminal or C-terminal directions when the heterologous Th epitope is covalently linked to the DPR fragment.

The heterologous Th epitope of the present disclosure can be any Th epitope that does not have an amino acid sequence naturally found in a DPR. The Th epitope can have an amino acid sequence derived from any species (e.g., human, pig, cattle, dog, rat, mouse, guinea pigs, etc.). The Th epitope can also have promiscuous binding motifs to MHC class II molecules of multiple species. In certain embodiments, the Th epitope comprises multiple promiscuous MHC class II binding motifs to allow maximal activation of T helper cells leading to initiation and regulation of immune responses. The Th epitope is preferably immunosilent on its own, i.e., little, if any, of the antibodies generated by the DPR peptide immunogen construct will be directed towards the Th epitope, thus allowing a very focused immune response directed to the targeted B cell epitope of the DPR.

Th epitopes of the present disclosure include, but are not limited to, amino acid sequences derived from foreign pathogens, as exemplified in Table 2 (SEQ ID NOs: 16 to 67). Further, the heterologous Th epitope can include an idealized artificial Th epitope in the form of either single sequence (e.g., SEQ ID NOs: 21, 22, 32, 33, and 43-46) or combinatorial sequences (e.g., SEQ ID NOs: 20, 25, 28, 31, 39, and 42). The heterologous Th epitope peptides presented as a combinatorial sequence (e.g., SEQ ID NOs: 20, 25, 28, 31, 39, and 42), contain a mixture of amino acid residues represented at specific positions within the peptide framework based on the variable residues of homologues for that particular peptide. An assembly of combinatorial peptides can be synthesized in one process by adding a mixture of the designated protected amino acids, instead of one particular amino acid, at a specified position during the synthesis process. Such combinatorial heterologous Th epitope peptides assemblies can allow broad Th epitope coverage for animals having a diverse genetic background. Representative combinatorial sequences of heterologous Th epitope peptides include SEQ ID NOs: 20, 25, 28, 31, 39, and 42, as shown in Table 2. Th epitope peptides of the present invention provide broad reactivity and immunogenicity to animals and patients from genetically diverse populations.

DPR peptide immunogen constructs comprising Th epitopes are produced simultaneously in a single solid-phase peptide synthesis in tandem with the DPR fragment. Th epitopes also include immunological analogues of known Th epitopes. Immunological Th analogues include immune-enhancing analogues, cross-reactive analogues, and segments of any of these Th epitopes that are sufficient to enhance or stimulate an immune response to the DPR fragments.

Functional immunologically analogues of the Th epitope peptides are also effective and included as part of the present disclosure. Functional immunological Th analogues can include conservative substitution in an amino acid position, a change in overall charge, a covalent attachment to another moiety, or amino acid additions, insertions, or deletions, and/or any combination thereof. Such immunologically functional analogues do not essentially modify the Th-stimulating function of the disclosed Th epitopes.

Conservative substitutions are when one amino acid residue is substituted for another amino acid residue with similar chemical properties. For example, the nonpolar (hydrophobic) amino acids include alanine, leucine, isoleucine, valine, proline, phenylalanine, tryptophan and methionine; the polar neutral amino acids include glycine, serine, threonine, cysteine, tyrosine, asparagine, and glutamine; the positively charged (basic) amino acids include arginine, lysine and histidine; and the negatively charged (acidic) amino acids include aspartic acid and glutamic acid.

The conservative substitutions, additions, and insertions can be accomplished with natural or non-natural amino acids. Non-naturally occurring amino acids include, but are not limited to, ε-N Lysine, β-alanine, ornithine, norleucine, norvaline, hydroxyproline, thyroxine, γ-amino butyric acid, homoserine, citrulline, aminobenzoic acid, 6-Aminocaproic acid (Aca; 6-Aminohexanoic acid), hydroxyproline, mercaptopropionic acid (MPA), 3-nitro-tyrosine, pyroglutamic acid, and the like. Naturally-occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

Table 2 identifies another variation of a functional analogue for Th epitope peptides. In particular, SEQ ID NOs: 21 and 22 of MvF1 and MvF2 Th are functional analogues of SEQ ID NOs: 31 and 32 of MvF4 and MvF5 in that they differ in the amino acid frame by the deletion (SEQ ID NOs: 21 and 22) or the inclusion (SEQ ID NOs: 31 and 32) of two amino acids each at the N- and C-termini. The differences between these two series of analogous sequences would not affect the function of the Th epitopes contained within these sequences. Therefore, functional immunological Th analogues include several versions of the Th epitope derived from Measles Virus Fusion protein MvF1-5 Ths (SEQ ID NOs: 21 to 33) and from Hepatitis Surface protein HBsAg 1-3 Ths (SEQ ID NOs: 34 to 46).

The Th epitope in the disclosed peptide immunogen construct can be covalently linked at the N- or C-terminal end, or both, of the DPR sequence. In some embodiments, the Th epitope is covalently linked to the N-terminal end of the DPR peptide. In other embodiments, the Th epitope is covalently linked to the C-terminal end of the DPR peptide. In certain embodiments, more than one Th epitope is covalently linked to the DPR fragment. When more than one Th epitope is linked to the DPR fragment, each Th epitope can have the same amino acid sequence or different amino acid sequences. In addition, when more than one Th epitope is linked to the DPR fragment, the Th epitopes can be arranged in any order. For example, the Th epitopes can be consecutively linked to the N-terminal end of the DPR fragment, or consecutively linked to the C-terminal end of the DPR fragment, or a Th epitope can be covalently linked to the N-terminal end of the DPR fragment while a separate Th epitope is covalently linked to the C-terminal end of the DPR fragment. There is no limitation in the arrangement of the Th epitopes in relation to the DPR fragment.

In some embodiments, the Th epitope is covalently linked to the DPR fragment directly. In other embodiments, the Th epitope is covalently linked to the DPR fragment through a heterologous spacer described in further detail below.

c. Heterologous Spacer

The disclosed peptide immunogen constructs optionally contain a heterologous spacer that covalently links the B cell epitope derived from a DPR protein to the heterologous T helper cell (Th) epitope.

As discussed above, the term “heterologous”, refers to an amino acid sequence that is derived from an amino acid sequence that is not part of, or homologous with, the wild-type sequence of the DPR. Thus, the natural amino acid sequence of the DPR is not extended in either the N-terminal or C-terminal directions when the heterologous spacer is covalently linked to the B cell epitope from DPR because the spacer is heterologous to the DPR sequence.

The spacer is any molecule or chemical structure capable of linking two amino acids and/or peptides together. The spacer can vary in length or polarity depending on the application. The spacer attachment can be through an amide- or carboxyl-linkage but other functionalities are possible as well. The spacer can include a chemical compound, a naturally occurring amino acid, or a non-naturally occurring amino acid.

The spacer can provide structural features to the peptide immunogen construct. Structurally, the spacer can provide a physical separation of the Th epitope from the B cell epitope of the DPR fragment. The physical separation by the spacer can disrupt any artificial secondary structures created by joining the Th epitope to the B cell epitope. Additionally, the physical separation of the B cell and Th epitopes by the spacer can eliminate interference between the Th cell and/or B cell responses. Furthermore, the spacer can be designed to create or modify a secondary structure of the peptide immunogen construct. For example, a spacer can be designed to act as a flexible hinge to enhance the separation of the Th epitope and B cell epitope. A flexible hinge spacer can also permit more efficient interactions between the presented peptide immunogen and the appropriate Th cells and B cells to enhance the immune responses to the Th epitope and B cell epitope. Examples of sequences encoding flexible hinges are found in the immunoglobulin heavy chain hinge region, which are often proline rich. One particularly useful flexible hinge that can be used as a spacer is provided by the sequence Pro-Pro-Xaa-Pro-Xaa-Pro (SEQ ID NO: 220), where Xaa is any amino acid, and preferably aspartic acid.

The spacer can also provide functional features to the peptide immunogen construct. For example, the spacer can be designed to change the overall charge of the peptide immunogen construct, which can affect the solubility of the peptide immunogen construct. Additionally, changing the overall charge of the peptide immunogen construct can affect the ability of the peptide immunogen construct to associate with other compounds and reagents. As discussed in further detail below, the peptide immunogen construct can be formed into a stable immunostimulatory complex with a highly charged oligonucleotide, such as CpG oligomers through electrostatic association. The overall charge of the peptide immunogen construct is important for the formation of these stable immunostimulatory complexes.

Chemical compounds that can be used as a spacer include, but are not limited to, (2-aminoethoxy) acetic acid (AEA), 5-aminovaleric acid (AVA), 6-aminocaproic acid (Ahx), 8-amino-3,6-dioxaoctanoic acid (AEEA, mini-PEG1), 12-amino-4,7,10-trioxadodecanoic acid (mini-PEG2), 15-amino-4,7,10,13-tetraoxapenta-decanoic acid (mini-PEG3), trioxatridecan-succinamic acid (Ttds), 12-amino-dodecanoic acid, Fmoc-5-amino-3-oxapentanoic acid (O1Pen), and the like.

Naturally-occurring amino acids that can be used as a spacer include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

Non-naturally occurring amino acids that can be used as a spacer include, but are not limited to, ε-N Lysine, β-alanine, ornithine, norleucine, norvaline, hydroxyproline, thyroxine, γ-amino butyric acid, homoserine, citrulline, aminobenzoic acid, 6-aminocaproic acid (Aca; 6-Aminohexanoic acid), hydroxyproline, mercaptopropionic acid (MPA), 3-nitro-tyrosine, pyroglutamic acid, and the like.

The spacer in the peptide immunogen construct can be covalently linked at the N-terminal end, the C-terminal end, or both ends of the DPR sequence. In some embodiments, the spacer is covalently linked to the C-terminal end of the Th epitope and to the N-terminal end of the DPR. In other embodiments, the spacer is covalently linked to the C-terminal end of the DPR and to the N-terminal end of the Th epitope. In certain embodiments, more than one spacer can be used, for example, when more than one Th epitope is present in the peptide immunogen construct. When more than one spacer is used, each spacer can be the same or different. Additionally, when more than one Th epitope is present consecutively in the peptide immunogen construct, the consecutive Th epitopes can be separated from each other with a spacer that is the same as, or different from, the spacer used to separate the Th epitope from the B cell epitope. There is no limitation in the arrangement of the spacer in relation to the Th epitope or the DPR fragment.

In certain embodiments, the heterologous spacer is a naturally occurring amino acid or a non-naturally occurring amino acid. In other embodiments, the spacer contains more than one naturally occurring or non-naturally occurring amino acid. In specific embodiments, the spacer is Lys-, Gly-, Lys-Lys-Lys-, (α, ε-N)Lys, ε-N-Lys-Lys-Lys-Lys (SEQ ID NO: 221), or Lys-Lys-Lys-ε-N-Lys (SEQ ID NO: 222).

d. Specific Embodiments of the DPR Peptide Immunogen Construct

In certain embodiments, the DPR peptide immunogen construct can be represented by the formula:

{(Th)ₘ-(A)ₙ-(DPR)-(A)ₙ-(Th)ₘ}_{y}-X

wherein

Th is a heterologous T helper epitope;

A is a heterologous spacer;

(DPR) is a B cell epitope having repeats of poly-GA, poly-GP, poly-GR, poly-PR, or poly-PA;

X is an α-COOH or α-CONH₂ of an amino acid;

the (DPR) has between about 4 to about 50 repeats of poly-GA, poly-GP, poly-GR, poly-PR, or poly-PA;

each m is from 0 to about 4;

each n is from 0 to about 10; and

y is from 1 to about 5.

In certain embodiments, the heterologous Th epitope in the peptide immunogen construct has an amino acid sequence selected from any of SEQ ID NOs: 16 to 67, or combinations thereof, shown in Table 2. In specific embodiments, the Th epitope has an amino acid sequence selected from any of SEQ ID NOs: 16 to 46. In certain embodiments, the peptide immunogen construct contains more than one Th epitope.

In certain embodiments, the optional heterologous spacer is selected from any of Lys-, Gly-, Lys-Lys-Lys-, (α, ε-N)Lys, ε-N-Lys-Lys-Lys-Lys (SEQ ID NO: 221), Lys-Lys-Lys-ε-N-Lys (SEQ ID NO: 222), and combinations thereof. In specific embodiments, the heterologous spacer is selected from any of (α, ε-N)Lys, ε-N-Lys-Lys-Lys-Lys (SEQ ID NO: 221), Lys-Lys-Lys-ε-N-Lys (SEQ ID NO: 222), and combinations thereof.

In certain embodiments, the DPR is a B cell epitope having about 10 to about 25 repeats of poly-GA, poly-GP, poly-GR, poly-PR, or poly-PA. In specific embodiments, the DPR contains 10, 15, or 25 repeats of poly-GA (SEQ ID NOs: 1-3), poly-GP (SEQ ID NOs: 4-5), poly-GR (SEQ ID NOs: 7-9), poly-PR (SEQ ID NOs: 10-12), or poly-PA (SEQ ID NOs: 13-15), as shown in Table 1.

In certain embodiments, the peptide immunogen construct contains about 10 to about 25 repeats of poly-GA, covalently linked to one or more Th epitope sequences through an optional spacer, as shown in Table 3. In other embodiments, the peptide immunogen construct contains about 10 to about 25 repeats of poly-GP, covalently linked to one or more Th epitope sequences through an optional spacer, as shown in Table 4. In certain embodiments, the peptide immunogen construct contains about 10 to about 25 repeats of poly-GR, covalently linked to one or more Th epitope sequences through an optional spacer, as shown in Table 5. In certain embodiments, the peptide immunogen construct contains about 10 to about 25 repeats of poly-PR, covalently linked to one or more Th epitope sequences through an optional spacer, as shown in Table 6. In certain embodiments, the peptide immunogen construct contains about 10 to about 25 repeats of poly-PA, covalently linked to one or more Th epitope sequences through an optional spacer, as shown in Table 7.

In specific embodiments, the peptide immunogen constructs have an amino acid sequence selected from the group consisting of SEQ ID NOs: 68, 69, 70, 80, 88, 98, 99, 110, 130, 148, 161, 173, 218, and 219, as shown in Table 9.

The peptide immunogen constructs of the present disclosure are capable of eliciting antibody production directed against the B cell epitope region of the constructs without activating an inflammatory T cell response.

### Compositions

The present disclosure also provides compositions comprising the disclosed peptide immunogen construct.

a. Peptide Compositions

Compositions containing a disclosed peptide immunogen construct can be in liquid or solid form. Liquid compositions can include water, buffers, solvents, salts, and/or any other acceptable reagent that does not alter the structural or functional properties of the peptide immunogen construct. Peptide compositions can contain one or more of the disclosed peptide immunogen constructs.

The composition can contain one peptide immunogen construct containing a single B cell epitope that contains repeats of poly-GA, poly-GP, poly-GR, poly-PR, or poly-PA. For example, in this embodiment, the composition can contain a peptide immunogen construct containing X repeats of either (a) poly-GA, (b) poly-GP, (c) poly-GR, (d) poly-PR, or (e) poly-PA, where X represents a number between 2 to 50.

The composition can also contain more than one peptide immunogen construct, where the peptide immunogen constructs in the composition differ by the length of the DRP, the sequence of the DPR, or both. In some embodiments, the composition can contain peptide immunogen constructs having 2, 3, 4, or 5 different DPR sequences as the B cell epitope i.e., compositions can contain any combination of peptide immunogen constructs containing (a) poly-GA, (b) poly-GP, (c) poly-GR, (d) poly-PR, and/or (e) poly-PA.

Non-limiting examples of compositions include:
a. A composition containing (1) a peptide immunogen construct having X repeats of either (a) poly-GA, (b) poly-GP, (c) poly-GR, (d) poly-PR, or (e) poly-PA together with (2) a separate peptide immunogen construct containing Y repeats of the same DPR, where X and Y represent a number between 2 to 50 and are not the same number.
b. A composition containing (1) a peptide immunogen construct containing X repeats of (a) poly-GA, (b) poly-GP, (c) poly-GR, (d) poly-PR, or (e) poly-PA and (2) a separate peptide immunogen construct containing Y repeats of (a) poly-GA, (b) poly-GP, (c) poly-GR, (d) poly-PR, or (e) poly-PA, where the peptide immunogen construct of (1) and (2) are different and X and Y represent a number between 2 and 50 that can be the same or different number.
c. A composition containing (1) a peptide immunogen construct containing V repeats of poly-GA; (2) a peptide immunogen construct containing W repeats of poly-GP; (3) a peptide immunogen construct containing X repeats of poly-GR; (4) a peptide immunogen construct containing Y repeats of poly-PR; (5) a peptide immunogen construct containing Z repeats of poly-PA, where V, W, X, Y, and Z each represent a number between 2 and 50 that can be the same or different number.

There is no limitation on the combination of peptide immunogen constructs that can be included in a peptide composition.

b. Pharmaceutical Compositions

The present disclosure is also directed to pharmaceutical compositions containing the disclosed peptide immunogen constructs.

Pharmaceutical compositions can contain carriers and/or other additives in a pharmaceutically acceptable delivery system. Accordingly, pharmaceutical compositions can contain a pharmaceutically effective amount of a peptide immunogen construct together with pharmaceutically-acceptable carrier, adjuvant, and/or other excipients such as diluents, additives, stabilizing agents, preservatives, solubilizing agents, buffers, and the like.

Pharmaceutical compositions can contain one or more adjuvant that act(s) to accelerate, prolong, or enhance the immune response to the peptide immunogen construct without having any specific antigenic effect itself. Adjuvants used in the pharmaceutical composition can include oils, aluminum salts, virosomes, aluminum phosphate (e.g. ADJU-PHOS®), aluminum hydroxide (e.g., ALHYDROGEL®), liposyn, saponin, squalene, L121, Emulsigen®, monophosphoryl lipid A (MPL), QS21, ISA 35, ISA 206, ISA50V, ISA51, ISA 720, as well as the other adjuvants and emulsifiers.

In some embodiments, the pharmaceutical composition contains MONTANIDE™ ISA 51 (an oil adjuvant composition comprised of vegetable oil and mannide oleate for production of water-in-oil emulsions), TWEEN® 80 (also known as: Polysorbate 80 or Polyoxyethylene (20) sorbitan monooleate), a CpG oligonucleotide, and/or any combination thereof. In other embodiments, the pharmaceutical composition is a water-in-oil-in-water (i.e., w/o/w) emulsion with EMULSIGEN or EMULSIGEN D as the adjuvant.

Pharmaceutical compositions can be formulated as immediate release or for sustained release formulations. Additionally the pharmaceutical compositions can be formulated for induction of systemic, or localized mucosal, immunity through immunogen entrapment and co-administration with microparticles. Such delivery systems are readily determined by one of ordinary skill in the art.

Pharmaceutical compositions can be prepared as injectables, either as liquid solutions or suspensions. Liquid vehicles containing the peptide immunogen construct can also be prepared prior to injection. The pharmaceutical composition can be administered by any suitable mode of application, for example, i.d., i.v., i.p., i.m., intranasally, orally, subcutaneously, etc. and in any suitable delivery device. In certain embodiments, the pharmaceutical composition is formulated for intravenous, subcutaneous, intradermal, or intramuscular administration. Pharmaceutical compositions suitable for other modes of administration can also be prepared, including oral and intranasal applications.

Pharmaceutical compositions can be formulated as immediate release or for sustained release formulations. Additionally the pharmaceutical compositions can be formulated for induction of systemic, or localized mucosal, immunity through immunogen entrapment and co-administration with microparticles. Such delivery systems are readily determined by one of ordinary skill in the art.

Pharmaceutical compositions can also formulated in a suitable dosage unit form. In some embodiments, the pharmaceutical composition contains from about 0.5 μg to about 1 mg of the peptide immunogen construct per kg body weight. Effective doses of the pharmaceutical compositions vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. When delivered in multiple doses, the pharmaceutical compositions may be conveniently divided into an appropriate amount per dosage unit form. The administered dosage will depend on the age, weight and general health of the subject as is well known in the therapeutic arts.

In some embodiments, the pharmaceutical composition contains more than one peptide immunogen construct. Similar to the peptide compositions described above, pharmaceutical compositions can contain more than one peptide immunogen construct, where the peptide immunogen constructs in the composition differ by the length of the DRP, the sequence of the DPR, or both. A pharmaceutical composition containing a mixture of more than one peptide immunogen construct to allow for synergistic enhancement of the immunoefficacy of the constructs. Pharmaceutical compositions containing more than one peptide immunogen construct can be more effective in a larger genetic population due to a broad MHC class II coverage thus provide an improved immune response to the peptide immunogen constructs.

In some embodiments, the pharmaceutical composition contains a peptide immunogen construct selected from SEQ ID NOs: 68, 69, 70, 80, 88, 98, 99, 110, 130, 148, 161, 173, as well as homologues, analogues and/or combinations thereof. Pharmaceutical compositions containing a peptide immunogen construct can be used to elicit an immune response and produce antibodies in a host upon administration.

c. Immunostimulatory Complexes

The present disclosure is also directed to pharmaceutical compositions containing a peptide immunogen construct in the form of an immunostimulatory complex with a CpG oligonucleotide. Such immunostimulatory complexes are specifically adapted to act as an adjuvant and as a peptide immunogen stabilizer. The immunostimulatory complexes are in the form of a particulate, which can efficiently present the peptide immunogen to the cells of the immune system to produce an immune response. The immunostimulatory complexes may be formulated as a suspension for parenteral administration. The immunostimulatory complexes may also be formulated in the form of w/o emulsions, as a suspension in combination with a mineral salt or with an in-situ gelling polymer for the efficient delivery of the peptide immunogen to the cells of the immune system of a host following parenteral administration.

The stabilized immunostimulatory complex can be formed by complexing a peptide immunogen construct with an anionic molecule, oligonucleotide, polynucleotide, or combinations thereof via electrostatic association. The stabilized immunostimulatory complex may be incorporated into a pharmaceutical composition as an immunogen delivery system.

In certain embodiments, the peptide immunogen construct is designed to contain a cationic portion that is positively charged at a pH in the range of 5.0 to 8.0. The net charge on the cationic portion of the peptide immunogen construct, or mixture of constructs, is calculated by assigning a +1 charge for each lysine (K), arginine (R) or histidine (H), a −1 charge for each aspartic acid (D) or glutamic acid (E) and a charge of 0 for the other amino acid within the sequence. The charges are summed within the cationic portion of the peptide immunogen construct and expressed as the net average charge. A suitable peptide immunogen has a cationic portion with a net average positive charge of +1. Preferably, the peptide immunogen has a net positive charge in the range that is larger than +2. In some embodiments, the cationic portion of the peptide immunogen construct is the heterologous spacer. In certain embodiments, the cationic portion of the peptide immunogen construct has a charge of +4 when the spacer sequence is (α, ε-N)Lys, ε-N-Lys-Lys-Lys-Lys (SEQ ID NO: 221), or Lys-Lys-Lys-ε-N-Lys (SEQ ID NO: 222).

An “anionic molecule” as described herein refers to any molecule that is negatively charged at a pH in the range of 5.0-8.0. In certain embodiments, the anionic molecule is an oligomer or polymer. The net negative charge on the oligomer or polymer is calculated by assigning a −1 charge for each phosphodiester or phosphorothioate group in the oligomer. A suitable anionic oligonucleotide is a single-stranded DNA molecule with 8 to 64 nucleotide bases, with the number of repeats of the CpG motif in the range of 1 to 10. In some embodiments, the CpG immunostimulatory single-stranded DNA molecules contain 18-48 nucleotide bases, with the number of repeats of CpG motif in the range of 3 to 8.

In certain embodiments, the anionic oligonucleotide is represented by the formula: 5′ X¹CGX² 3′ wherein C and G are unmethylated; and X¹ is selected from the group consisting of A (adenine), G (guanine) and T (thymine); and X² is C (cytosine) or T (thymine). Or, the anionic oligonucleotide is represented by the formula: 5′ (X³)₂CG(X⁴)₂ 3′ wherein C and G are unmethylated; and X³ is selected from the group consisting of A, T or G; and X⁴ is C or T. In specific embodiments, the CpG oligonucleotide is selected from a group consisting of 5′ TCG TCG TTT TGT CGT TTT GTC GTT TTG TCG TT 3′ (CpG1) SEQ ID NO: 223, a 32 base length oligomer, and 5′nTC GTC GTT TTG TCG TTT TGT CGT T 3′ (CpG2) SEQ ID NO: 224, a 24 base length oligomer plus an phosphorothioate group (designated as n at the 5′ end).

The resulting immunostimulatory complex is in the form of particles with a size typically in the range from 1-50 microns and is a function of many factors including the relative charge stoichiometry and molecular weight of the interacting species. The particulated immunostimulatory complex has the advantage of providing adjuvantation and upregulation of specific immune responses in vivo. Additionally, the stabilized immunostimulatory complex is suitable for preparing pharmaceutical compositions by various processes including water-in-oil emulsions, mineral salt suspensions and polymeric gels.

### Antibodies

The present disclosure also provides antibodies elicited by the peptide immunogen construct.

The disclosed peptide immunogen constructs, comprising a DPR fragment, heterologous Th epitope, and optional heterologous spacer, are capable of eliciting an immune response and the production of antibodies when administered to a host. The design of the peptide immunogen constructs can break tolerance to self-proteins and elicit the production of site-specific antibodies.

The disclosed antibodies bind with high specificity to the respective DPR without much, if any, directed to the heterologous Th epitopes employed for immunogenicity enhancement, which is in sharp contrast to the conventional protein or other biological carriers used for such peptide antigenicity enhancement.

The disclosed antibodies can be used in assays to detect the presence of DPR proteins in a sample (e.g., cerebral spinal fluid, CSF or a tissue).

### Methods

The present disclosure is also directed to methods for making and using the peptide immunogen constructs, compositions, and pharmaceutical compositions.

a. Methods for Manufacturing the Peptide Immunogen Construct

The peptide immunogen constructs of this disclosure can be made by chemical synthesis methods well known to the ordinarily skilled artisan (see, e.g., Fields et al., Chapter 3 in Synthetic Peptides: A User's Guide, ed. Grant, W. H. Freeman & Co., New York, N.Y., 1992, p. 77). The peptide immunogen constructs can be synthesized using the automated Merrifield techniques of solid phase synthesis with the α-NH₂ protected by either t-Boc or F-moc chemistry using side chain protected amino acids on, for example, an Applied Biosystems Peptide Synthesizer Model 430A or 431. Preparation of peptide immunogen constructs comprising combinatorial library peptides for Th epitopes can be accomplished by providing a mixture of alternative amino acids for coupling at a given variable position.

After complete assembly of the desired peptide immunogen construct, the resin can be treated according to standard procedures to cleave the peptide from the resin and the functional groups on the amino acid side chains can be deblocked. The free peptide can be purified by HPLC and characterized biochemically, for example, by amino acid analysis or by sequencing. Purification and characterization methods for peptides are well known to one of ordinary skill in the art.

The quality of peptides produced by this chemical process can be controlled and defined and, as a result, reproducibility of peptide immunogen constructs, immunogenicity, and yield can be assured. Detailed description of the manufacturing of the peptide immunogen construct through solid phase peptide synthesis is shown in Example 1.

The range in structural variability that allows for retention of an intended immunological activity has been found to be far more accommodating than the range in structural variability allowed for retention of a specific drug activity by a small molecule drug or the desired activities and undesired toxicities found in large molecules that are co-produced with biologically-derived drugs. Thus, peptide analogues, either intentionally designed or inevitably produced by errors of the synthetic process as a mixture of deletion sequence byproducts that have chromatographic and immunologic properties similar to the intended peptide, are frequently as effective as a purified preparation of the desired peptide. Designed analogues and unintended analogue mixtures are effective as long as a discerning QC procedure is developed to monitor both the manufacturing process and the product evaluation process so as to guarantee the reproducibility and efficacy of the final product employing these peptides.

The peptide immunogen constructs can also be made using recombinant DNA technology including nucleic acid molecules, vectors, and/or host cells. As such, nucleic acid molecules encoding the peptide immunogen construct and immunologically functional analogues thereof are also encompassed by the present disclosure as part of the present invention. Similarly, vectors, including expression vectors, comprising nucleic acid molecules as well as host cells containing the vectors are also encompassed by the present disclosure as part of the present invention.

Various exemplary embodiments also encompass methods of producing the peptide immunogen construct and immunologically functional analogues thereof. For example, methods can include a step of incubating a host cell containing an expression vector containing a nucleic acid molecule encoding a peptide immunogen construct and/or immunologically functional analogue thereof under such conditions where the peptide and/or analogue is expressed. The longer synthetic peptide immunogens can be synthesized by well-known recombinant DNA techniques. Such techniques are provided in well-known standard manuals with detailed protocols. To construct a gene encoding a peptide of this invention, the amino acid sequence can be reverse translated to obtain a nucleic acid sequence encoding the amino acid sequence, preferably with codons that are optimum for the organism in which the gene is to be expressed. Next, a synthetic gene is made typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

b. Methods for the Manufacturing of Immunostimulatory Complexes

Various exemplary embodiments also encompass methods of producing the Immunostimulatory complexes comprising the peptide immunogen constructs and CpG oligodeoxynucleotide (ODN) molecule. Stabilized immunostimulatory complexes (ISC) are derived from a cationic portion of the peptide immunogen construct and a polyanionic CpG ODN molecule. The self-assembling system is driven by electrostatic neutralization of charge. Stoichiometry of the molar charge ratio of cationic portion of the peptide immunogen construct to anionic oligomer determines extent of association. The non-covalent electrostatic association of peptide immunogen construct and CpG ODN is a completely reproducible process. The peptide/CpG ODN immunostimulatory complex aggregates, which facilitate presentation to the “professional” antigen-presenting cells (APC) of the immune system thus further enhancing of the immunogenicity of the complexes. These complexes are easily characterized for quality control during manufacturing. The peptide/CpG ISC are well tolerated in vivo. This particulate system comprising a CpG ODN and a peptide immunogen construct is designed to take advantage of the generalized B cell mitogenicity associated with CpG ODN use, yet promote balanced Th-1/Th-2 type responses.

The CpG ODN in the disclosed pharmaceutical compositions is 100% bound to immunogen in a process mediated by electrostatic neutralization of opposing charge, resulting in the formation of micron-sized particulates. The particulate form allows for a significantly reduced dosage of CpG from the conventional use of CpG adjuvants, less potential for adverse innate immune responses, and facilitates alternative immunogen processing pathways including antigen-presenting cells (APC). Consequently, such formulations are novel conceptually and offer potential advantages by promoting the stimulation of immune responses by alternative mechanisms.

c. Methods for Manufacturing Pharmaceutical Compositions

Various exemplary embodiments also encompass pharmaceutical compositions containing peptide immunogen constructs. In certain embodiments, the pharmaceutical compositions employ water in oil emulsions and in suspension with mineral salts.

In order for a pharmaceutical composition to be used by a large population and with the clearance of DPR proteins also being part of the goal for administration, safety becomes another important factor for consideration. Despite the use of water-in-oil emulsions in humans for many formulations in clinical trials, Alum remains the major adjuvant for use in formulations due to its safety. Alum or its mineral salts Aluminum phosphate (ADJUPHOS) are, therefore, frequently used as adjuvants in preparation for clinical applications.

d. Methods Using Pharmaceutical Compositions

The present disclosure also includes methods of using pharmaceutical compositions containing peptide immunogen constructs.

In certain embodiments, the pharmaceutical compositions containing peptide immunogen constructs can be used to clear DPR proteins from a subject. This method comprises administering a pharmaceutical composition comprising a pharmacologically effective amount of a peptide immunogen construct to a host in need thereof.

### Specific Embodiments

Specific embodiments of the present invention include, but are not limited to, the following:
(1) A dipeptide repeat (DPR) peptide immunogen construct comprising:
   a B cell epitope comprising about 10 to about 25 repeats of poly-GA, poly-GP, poly-GR, poly-PR, or poly-PA;
   a heterologous T helper epitope comprising an amino acid sequence selected from the group consisting of SEQ ID NOs: 16 to 67; and
   an optional heterologous spacer selected from the group consisting of an amino acid, Lys-, Gly-, Lys-Lys-Lys-, (α, ε-N)Lys, ε-N-Lys-Lys-Lys-Lys (SEQ ID NO: 221), Lys-Lys-Lys-ε-N-Lys (SEQ ID NO: 222); and
   wherein the B cell epitope is covalently linked to the T helper epitope directly or through the optional heterologous spacer.
(2) The DPR peptide immunogen construct of (1), wherein
   the repeats of poly-GA have an amino acid sequence of SEQ ID NOs: 1, 2, or 3; and
   the repeats of poly-GP have an amino acid sequence of SEQ ID NOs: 4, 5, or 6; and
   the repeats of poly-GR have an amino acid sequence of SEQ ID NOs: 7, 8, or 9; and
   the repeats of poly-PR have an amino acid sequence of SEQ ID NOs: 10, 11, or 12; and
   the repeats of poly-PA have an amino acid sequence of SEQ ID NOs: 13, 14, or 15.
(3) The DPR peptide immunogen construct of (1), wherein the amino acid sequence of the heterologous T helper epitope is selected from the group consisting of SEQ ID NO: 31, 32, and a combination thereof
(4) The DPR peptide immunogen construct of (1), wherein the optional heterologous spacer is (α, ε-N)Lys, ε-N-Lys-Lys-Lys-Lys (SEQ ID NO: 221), or Lys-Lys-Lys-ε-N-Lys (SEQ ID NO: 222).
(5) The DPR peptide immunogen construct of (1) comprising the following formula:

{(Th)ₘ-(A)ₙ-(DPR)-(A)ₙ-(Th)ₘ}_{y}-X
wherein
Th is the heterologous T helper epitope;
A is the heterologous spacer;
(DPR) is a B cell epitope having repeats of poly-GA, poly-GP, poly-GR, poly-PR, or poly-PA;
X is an α-COOH or α-CONH₂ of an amino acid;
each m is from 0 to about 4;
each n is from 0 to about 10; and
y is from 1 to about 5.
(6) The DPR peptide immunogen construct of (5), wherein
   the repeats of poly-GA have an amino acid sequence of SEQ ID NOs: 1, 2, or 3; and
   the repeats of poly-GP have an amino acid sequence of SEQ ID NOs: 4, 5, or 6; and
   the repeats of poly-GR have an amino acid sequence of SEQ ID NOs: 7, 8, or 9; and
   the repeats of poly-PR have an amino acid sequence of SEQ ID NOs: 10, 11, or 12; and
   the repeats of poly-PA have an amino acid sequence of SEQ ID NOs: 13, 14, or 15.
(7) The DPR peptide immunogen construct of (5), wherein the amino acid sequence of the heterologous T helper epitope is selected from the group consisting of SEQ ID NO: 31, 32, and a combination thereof
(8) The DPR peptide immunogen construct of (5), wherein the optional heterologous spacer is (α, ε-N)Lys, ε-N-Lys-Lys-Lys-Lys (SEQ ID NO: 221), or Lys-Lys-Lys-ε-N-Lys (SEQ ID NO: 222).
(9) The DPR peptide immunogen construct of (1) comprising an amino acid sequence selected from the group consisting of SEQ ID NOs: 68 to 219, and any combination thereof
(10) The DPR peptide immunogen construct of (1) comprising an amino acid sequence selected from the group consisting of 68, 69, 70, 80, 88, 98, 99, 110, 130, 148, 161, 173, 218, 219, and any combination thereof
(11) A composition comprising the DPR peptide immunogen construct of (1).
(12) A composition comprising more than one peptide immunogen construct of (1).
(13) The composition of (11), wherein the DPR peptide immunogen construct has amino acid sequence selected from the group consisting of SEQ ID NOs: 68 to 219, and any combination thereof
(14) A pharmaceutical composition comprising the DPR peptide immunogen construct of (1) and a pharmaceutically acceptable delivery vehicle and/or adjuvant.
(15) The pharmaceutical composition of (14), wherein
   a. the DPR peptide immunogen construct is selected from the group consisting of SEQ ID NOs: 68 to 219, and any combination thereof and
   b. the adjuvant is a mineral salt of aluminum selected from the group consisting of Al(OH)₃ or AlPO₄.
(16) The pharmaceutical composition of (14), wherein
   a. the DPR peptide immunogen construct is selected from the group consisting of SEQ ID NOs: 68 to 219, and any combination thereof and
   b. the DPR peptide immunogen construct is mixed with an CpG oligodeoxynucleotide (ODN) to form a stabilized immunostimulatory complex.
(17) An isolated antibody or epitope-binding fragment thereof that specifically binds to the B cell epitope of the DPR peptide immunogen construct of (1).
(18) The isolated antibody or epitope-binding fragment thereof according to (17) bound to the DPR peptide immunogen construct.
(19) An isolated antibody or epitope-biding fragment thereof that specifically binds to the B cell epitope of the DPR peptide immunogen construct of (9).
(20) A composition comprising the isolated antibody or epitope-binding fragment thereof according to (17).
(21) A method for producing antibodies that recognize DPR proteins in a host comprising administering to the host a composition comprising the DPR peptide immunogen construct of (1) and a delivery vehicle and/or adjuvant.
(22) A method for reducing the amount of DPR proteins in an animal comprising administering a pharmacologically effective amount of the DPR peptide immunogen of (1) to the animal.
(23) The method of (22), wherein the animal is a human.
(24) A method for identifying DPR proteins in a biological sample comprising:
   a. exposing the biological sample to the antibody or epitope-binding fragment thereof according to (17) under conditions that allow the antibody or epitope-binding fragment thereof to bind to a DPR protein; and
   b. detecting the amount of the antibody or epitope-binding fragment thereof bound to the DPR protein in the biological sample.

### Example 1

### Synthesis of DPR Peptide Immunogen Constructs and Preparation of Formulations Thereof

a. Synthesis of DPR Peptide Immunogen Constructs

Methods for synthesizing DPR peptide immunogen constructs are described. The peptides were synthesized in small-scale amounts that are useful for serological assays, laboratory pilot and field studies. The peptides can also be produced in large-scale (kilogram) amounts, which are useful for industrial/commercial production of pharmaceutical compositions. DPR peptides containing 10, 15, and/or 25 repeats of poly-GA, poly-GP, poly-GR, or poly-PR were prepared, which sequences are shown in Table 1.

Selected DPR fragments were made into DPR peptide immunogen constructs by synthetically linking the fragments to one or more carefully designed helper T cell (Th) epitope derived from the pathogen protein Measles Virus Fusion protein. Specifically, the DPR fragments were linked to MvF5 Th (UBITh®1, SEQ ID NO: 32) or MvF4 Th (UBITh®3, SEQ ID NO: 31), which sequences are shown in Table 2.

Representative DPR peptide immunogen constructs, selected from over 100 peptide constructs, were prepared and are identified in Table 9 (SEQ ID NOs: 68, 69, 70, 80, 88, 98, 99, 110, 130, 148, 161, 173, 218, and 219). All peptides used for immunogenicity studies or related serological tests for detection and/or measurement of DPR antibodies were synthesized in small scale using F-moc chemistry by peptide synthesizers of Applied BioSystems Models 430A, 431 and/or 433. Each peptide was produced by an independent synthesis on a solid-phase support, with F-moc protection at the N-terminus and side chain protecting groups of trifunctional amino acids. Completed peptides were cleaved from the solid support and side chain protecting groups were removed by 90% trifluoroacetic acid (TFA). Synthetic peptide preparations were evaluated by Matrix-Assisted Laser Desorption/Ionization-Time-Of-Flight (MALDI-TOF) Mass Spectrometry to ensure correct amino acid content. Each synthetic peptide was also evaluated by Reverse Phase HPLC (RP-HPLC) to confirm the synthesis profile and concentration of the preparation. Despite rigorous control of the synthesis process (including stepwise monitoring the coupling efficiency), peptide analogues were also produced due to unintended events during elongation cycles, including amino acid insertion, deletion, substitution, and premature termination. Thus, synthesized preparations typically included multiple peptide analogues along with the targeted peptide. Despite the inclusion of such unintended peptide analogues, the resulting synthesized peptide preparations were nevertheless suitable for use in immunological applications including immunodiagnosis (as antibody capture antigens) and pharmaceutical compositions (as peptide immunogens). Typically, such peptide analogues, either intentionally designed or generated through synthetic process as a mixture of byproducts, are frequently as effective as a purified preparation of the desired peptide, as long as a discerning QC procedure is developed to monitor both the manufacturing process and the product evaluation process to guarantee the reproducibility and efficacy of the final product employing these peptides.

b. Preparation of Compositions Containing DPR Peptide Immunogen Constructs

Formulations employing water in oil emulsions and in suspension with mineral salts were prepared.

Briefly, DPR peptide immunogen constructs were prepared (i) in a water-in-oil emulsion with Seppic Montanide™ ISA 51 as the approved oil for human use, or (ii) mixed with mineral salts ADJUPHOS (Aluminum phosphate) or ALHYDROGEL (Alum), at varying amounts of peptide constructs, as specified. Compositions were typically prepared by dissolving the DPR peptide immunogen constructs in water at about 20 to 800 μg/mL and formulated with Montanide™ ISA 51 into water-in-oil emulsions (1:1 in volume) or with mineral salts or ALHYDROGEL (Alum) (1:1 in volume). The compositions were kept at room temperature for about 30 min and mixed by vortex for about 10 to 15 seconds prior to immunization. Some animals were immunized with 2 to 3 doses of a specific composition, which were administered at time 0 (prime) and 3 week post initial immunization (wpi) (booster), optionally 5 or 6 wpi for a second boost, by intramuscular route. These immunized animals were then tested with selected B cell epitope peptide(s) to evaluate the immunogenicity of the various peptide immunogen constructs present in the formulation as well as their cross-reactivity with related target peptides or proteins.

### Example 2

### Antibody Titers Obtained from Immunizations with DPR Peptide Immunogen Constructs

The immunization and evaluation of various DPR peptide immunogen constructs are described in detail below.

a. Immunizations and Sera Collection

The formulations specified in each of the study groups generally contained all types of designer DPR peptide immunogen constructs with a segment of the DPR B cell epitope peptide linked via different type of spacers (e.g., εLys (εK) or Lysine-lysine-lysine (KKK) to enhance the peptide construct's solubility) and promiscuous helper T cell epitopes including two sets of artificial T helper epitopes derived from Measles virus fusion protein and Hepatitis B surface antigen. The DPR B cell epitope peptides were linked at the N- or C-terminus of the designer peptide constructs. DPR peptide immunogen constructs were initially evaluated in guinea pigs for their relative immunogenicity with the corresponding DPR B cell epitope peptides or peptide immunogens.

Each peptide immunogen was formulated in MONTANIDE™ ISA51 and CpG to immunize guinea pigs at dose at 400 μg/ml as prime immunization and 100 μg/ml as boost dose at 3, 6, 9 weeks post-injection (WPI), 3 guinea pigs per group.

b. Evaluation of Antibody Titers

ELISA assay was performed to evaluate the immunogenicity of the designer DPR peptide immunogen constructs. DPR B cell epitope peptides or peptide immunogen constructs were used to coat the plate wells, which served as targeting peptides. Guinea pig immune serum was diluted from 1:100 to 1:100,000 by 10-fold serial dilutions. The titer of a tested serum, expressed as Log 10, was calculated by linear regression analysis of the A450 nm with the cut off A₄₅₀ set at 0.5. All peptide immunogens induced strong immunogenicity titers against the B epitope peptides coated in the plate wells.

### Example 3

### Serological Assays and Reagents

Serological assays and reagents for evaluating functional immunogenicity of the synthetic peptide constructs and formulations thereof are described in detail below.

a. Peptide-Based ELISA Tests for Antibody Specificity Analysis

ELISA assays for evaluating immune serum samples were developed as described below.

The wells of 96-well plates were coated individually for 1 hour at 37° C. with 100 μL of target peptide DPR fragments or peptide constructs (SEQ ID NOs: 10, 68-70, 88, 98, 99, 130, 148), at 2 μg/mL (unless noted otherwise), in 10 mM NaHCO₃ buffer, pH 9.5 (unless noted otherwise).

b. Assessment of Antibody Reactivity Towards DPRs by ELISA Tests

The peptide-coated wells (SEQ ID NOs: 10, 68-70, 88, 98, 99, 130, and 148) were incubated with 250 μL of 3% by weight of gelatin in PBS in 37° C. for 1 hour to block non-specific protein binding sites, followed by three washes with PBS containing 0.05% by volume of TWEEN® 20 and dried. Sera to be analyzed were diluted 1:20 (unless noted otherwise) with PBS containing 20% by volume normal goat serum, 1% by weight gelatin and 0.05% by volume TWEEN® 20. One hundred microliters (100 μL) of the diluted specimens (e.g., serum, plasma) were added to each of the wells and allowed to react for 60 minutes at 37° C. The wells were then washed six times with 0.05% by volume TWEEN® 20 in PBS in order to remove unbound antibodies. Horseradish peroxidase (HRP)-conjugated species (e.g., mouse, guinea pig, or human) specific goat anti-IgG, was used as a labeled tracer to bind with the antibody/peptide antigen complex formed in positive wells. One hundred microliters of the peroxidase-labeled goat anti-IgG, at a pre-titered optimal dilution and in 1% by volume normal goat serum with 0.05% by volume TWEEN® 20 in PBS, was added to each well and incubated at 37° C. for another 30 minutes. The wells were washed six times with 0.05% by volume TWEEN® 20 in PBS to remove unbound antibody and reacted with 100 μL of the substrate mixture containing 0.04% by weight 3′, 3′, 5′, 5′-Tetramethylbenzidine (TMB) and 0.12% by volume hydrogen peroxide in sodium citrate buffer for another 15 minutes. This substrate mixture was used to detect the peroxidase label by forming a colored product. Reactions were stopped by the addition of 100 μL of 1.0M H₂SO₄ and absorbance at 450 nm (A₄₅₀) determined. For the determination of antibody titers of the immunized animals that received the various DPR derived peptide immunogens, 10-fold serial dilutions of sera from 1:100 to 1:10,000 were tested, and the titer of a tested serum, expressed as Log₁₀, was calculated by linear regression analysis of the A₄₅₀ with the cutoff A₄₅₀ set at 0.5.

c. Immunogenicity Evaluation

Preimmune and immune serum samples from animals were collected according to experimental immunization protocols and heated at 56° C. for 30 minutes to inactivate serum complement factors. Following the administration of the pharmaceutical composition containing a DPR peptide immunogen construct, blood samples were obtained according to protocols and their immunogenicity against specific target site(s) evaluated. Serially diluted sera were tested and positive titers were expressed as Log₁₀ of the reciprocal dilution. Immunogenicity of a particular pharmaceutical composition is assessed by its ability to elicit high titer B cell antibody response directed against the desired epitope specificity within the target antigen while maintaining a low to negligible antibody reactivity towards the “Helper T cell epitopes” employed to provide enhancement of the desired B cell responses.

d. Immunoassay for DPR Level in Mouse Immune Sera

Serum DPR levels in mice receiving DPR derived peptide immunogens were measured by a sandwich ELISA (Cloud-don, SEB222Mu) using anti-DPR antibodies as capture antibody and biotin-labeled anti-DPR antibody as detection antibody. Briefly, the antibody was immobilized on 96-well plates at 100 ng/well in coating buffer (15 mM Na₂CO₃, 35 mM NaHCO₃, pH 9.6) and incubated at 4° C. overnight. Coated wells were blocked with 200 μL/well of assay diluents (0.5% BSA, 0.05% TWEEN®-20, 0.02% ProClin 300 in PBS) at room temperature for 1 hour. Plates were washed 3 times with 200 μL/well of wash buffer (PBS with 0.05% TWEEN®-20). Purified recombinant DPR was used to generate a standard curve (range 156 to 1,250 ng/mL by 2-fold serial dilution) in assay diluent with 5% mouse sera. Fifty microliters (50 μL) of the diluted sera (1:20) and standards were added to coated wells. The incubation was carried out at room temperature for 1 hour. All wells were aspirated and washed 6 times with 200 μL/well of wash buffer. The captured DPR was incubated with 100 μL of detection antibody solution (50 ng/ml of biotin labeled HP6029 in assay diluent) at room temperature for 1 hour. Then, the bound biotin-HP6029 was detected using streptavidin poly-HRP (1:10,000 dilution, Thermo Pierce) for 1 hour (100 μL/well). All wells were aspirated and washed 6 times with 200 μL/well of wash buffer and the reaction was stopped by addition of 100 μL/well of 1M H₂SO₄. The standard curve was created by using the SoftMax Pro software (Molecular Devices) to generate a four parameter logistic curve-fit and used to calculate the concentrations of DPR in all tested samples. Student t tests were used to compare data by using the Prism software.

e. Purification of Anti-DPR Antibodies

Anti-DPR antibodies were purified from sera collected at 3 to 15 weeks post-injection (WPI) of guinea pigs or mice immunized with DPR peptide immunogen constructs containing peptides of different sequences (SEQ ID NOs: 68, 69, 70, 80, 88, 98, 99, 110, 130, 148, 161, and 173) by using an affinity column (Thermo Scientific, Rockford). Briefly, after buffer (0.1 M phosphate and 0.15 M sodium chloride, pH 7.2) equilibration, 400 μL of serum was added into the Nab Protein G Spin column followed by end-over-end mixing for 10 min and centrifugation at 5,800×g for 1 min. The column was washed with binding buffer (400 μL) for three times. Subsequently, elution buffer (400 μL, 0.1 M glycine pH 2.0) was added into the spin column to elute the antibodies after centrifuging at 5,800×g for 1 min. The eluted antibodies were mixed with neutralization buffer (400 μL, 0.1 M Tris pH 8.0) and the concentrations of these purified antibodies were measured by using Nan-Drop at OD280, with BSA (bovine serum albumin) as the standard.

f. Results

The immunogenicity titer against DPR peptides or peptide immunogens from the immunized guinea pig serum was assessed by ELISA.

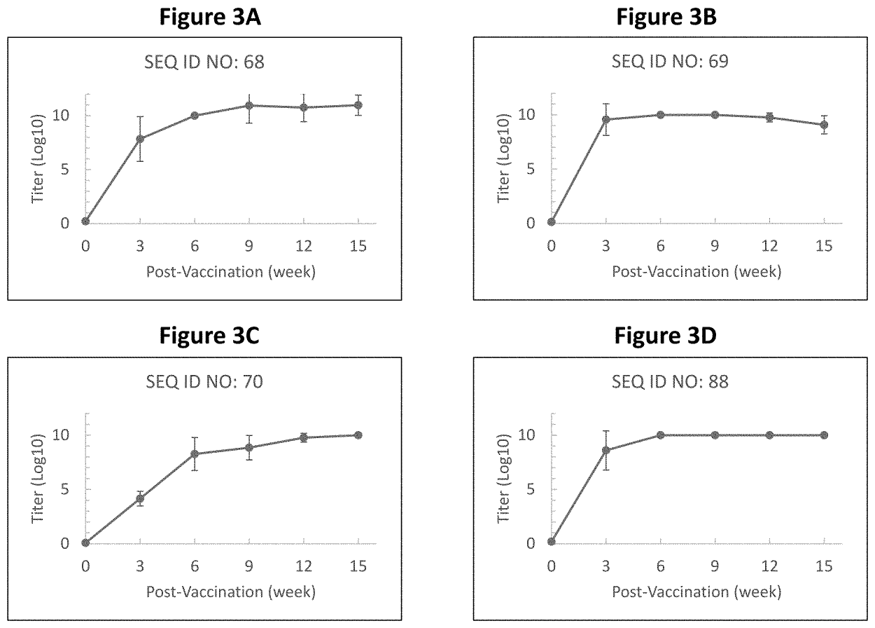

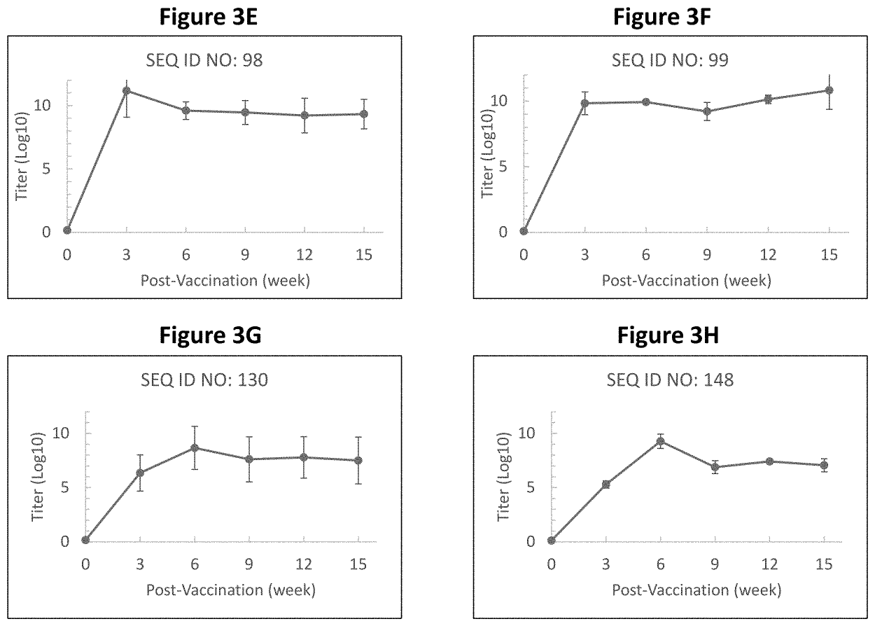

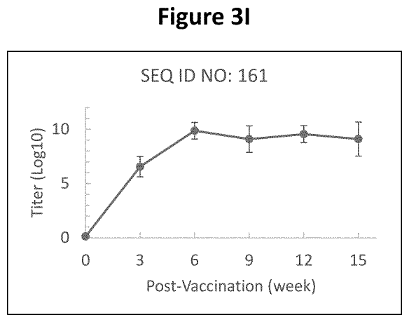

Tables 10-11 and FIGS. 3A-3I show the properties of antisera over a 15-week period in guinea pigs immunized with 12 different DPR peptide immunogen constructs. The guinea pig antisera from 0, 3, 6, 9, 12 and 15 wpi were diluted by a 10-fold serial dilution. ELISA plates were coated with DPR peptides or peptide immunogens. The titer of a tested serum, expressed as Log₁₀, was calculated by linear regression analysis of the A₄₅₀ nm with the cutoff A₄₅₀ set at 0.5.

The ELISA data for DPR peptide immunogen constructs containing poly-GA, poly-GP, and poly-GR peptides (SEQ ID NOs: 68-70, 80, 88, 98, 99, 110, 130, and 148) is shown in Table 10 and the ELISA data for DPR peptide immunogen constructs containing poly-PR (SEQ ID NOs: 161 and 173) is shown in Table 11.

The ELISA data were then plotted as graphs shown in FIGS. 3A-3I, where the same peptide immunogen that was used to immunize the animal was bound to the ELISA plate for analysis. Specifically, the antibody titers obtained after immunization with poly-GA constructs (SEQ ID NOs: 68, 69, 70, and 88) are shown in FIGS. 3A-3D, respectively. Antibody titers obtained after immunization with poly-GP constructs (SEQ ID NOs: 98 and 99) are shown in FIGS. 3E-3F, respectively. Antibody titers obtained after immunization with poly-GR constructs (SEQ ID NOs: 130 and 148) are shown in FIGS. 3G-3H, respectively. Antibody titers obtained after immunization with a poly-GR construct (SEQ ID NO: 161) is shown in FIG. 3I.

All of DPR immunogen constructs, demonstrated high immunogenicity to the corresponding DPR peptides or peptide immunogens. The ELISA results showed no detectable antibody titer was observed in each group prior to immunization at week 0. After three immunizations, the titer peaked in each group at week 6 with lop) titers mostly higher than 12, and remained in plateau through the study termination at week 15 (Tables 10-11). The data show that the length of dipeptide repeats in the DPR peptide immunogen constructs do not appear to have much of effect on antibody titers. For example, poly-GA 10, 15 and 25 repeats constructs (SEQ ID NO: 68-70, 80, and 88) display very similar immunogenicity, as shown in Table 10 and FIGS. 3A-3D.

Interestingly, peptide immunogen constructs containing poly-GA (SEQ ID NOs: 68-70, 80, and 88), poly-GP (SEQ ID NOs: 98, 99, and 110), and poly-GR (SEQ ID NOs: 130 and 148) not only displayed high immunogenicity to their corresponding peptide immunogens, but also were able to elicit a certain extent of cross-reactivity to each of the other peptide immunogen constructs (see Table 10). For example, Table 10 shows that all of the DPR peptide immunogen constructs containing poly-GA (SEQ ID NO: 68-70, 80, 88) generated antibody titers against poly-GP (SEQ ID NO: 98, 99 and 110) and poly-GR constructs (SEQ ID NO: 130 and 148). Also, the DPR peptide immunogen constructs containing poly-GR show similar cross-reactivity to poly-GA and poly-GP. However, the DPR peptide immunogen constructs containing poly-GP (SEQ ID NOs: 98, 99, and 110) had a lower cross-reactivity to poly-GR constructs.

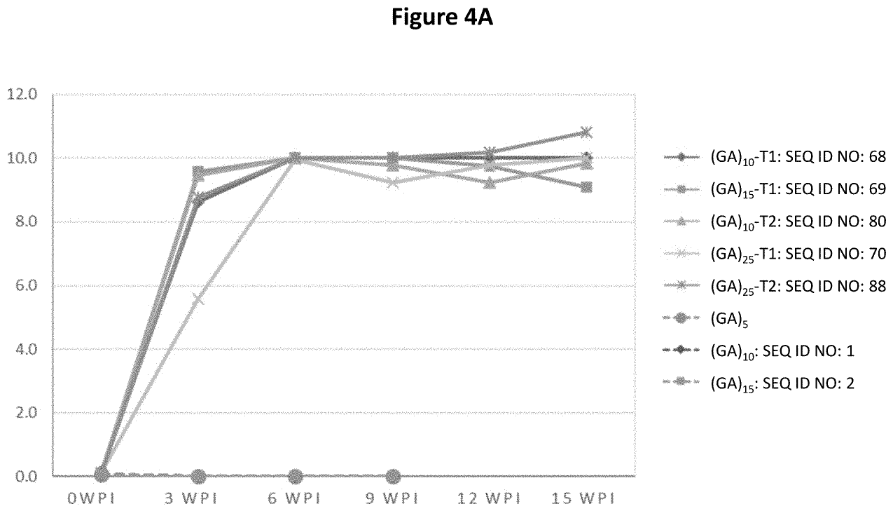

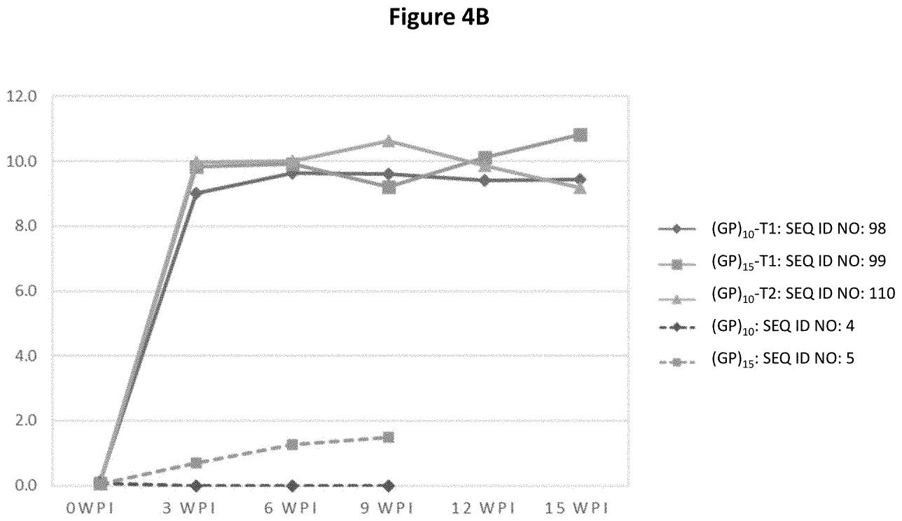

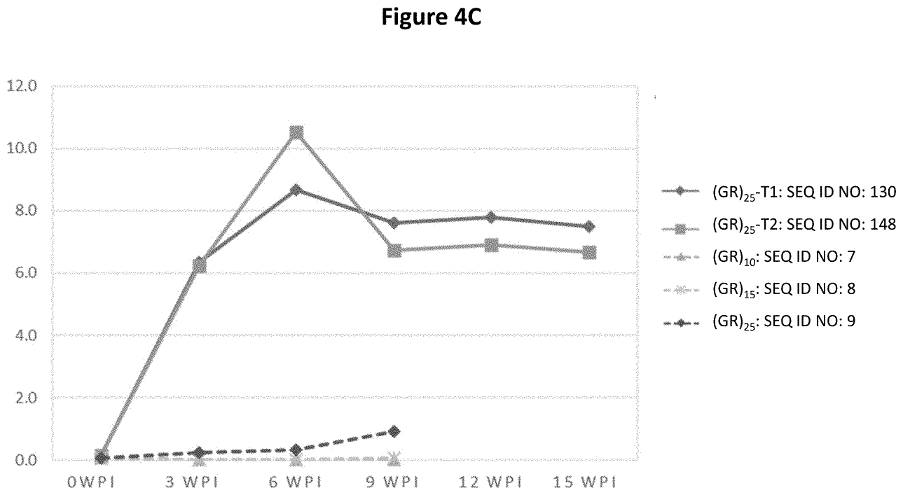

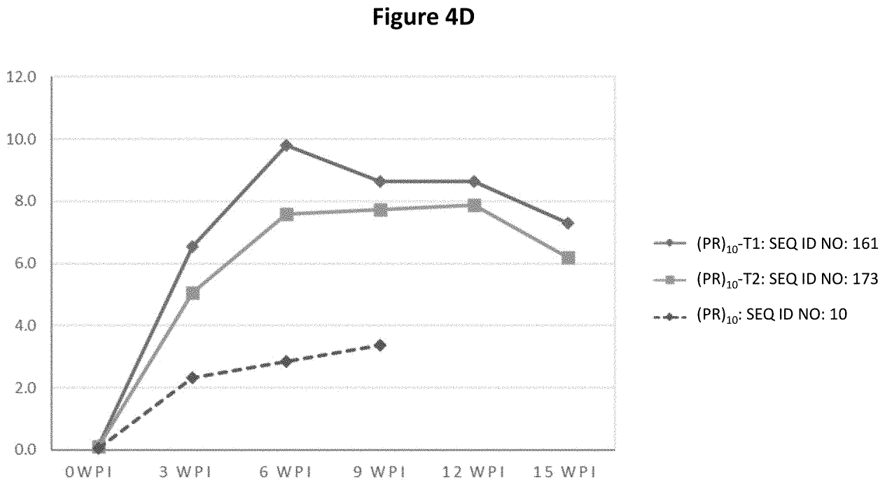

The immunogenicity of the DPR peptide immunogen constructs were also compared with the corresponding target B cell epitope peptides, as shown in FIGS. 4A-4D.

The antibody titers obtained after immunization with poly-GA constructs linked to a Th epitope (SEQ ID NOs: 68, 69, 70, 80 and 88) and poly-GA constructs not linked to a Th epitope (GA)₅ or (GA)₁₀ and (GA)₁₅ (SEQ ID NOs: 1 and 2) were analyzed by ELISA against the (GA)₂₅-KKK-εK-UBITh®1 construct of SEQ ID NO: 70. FIG. 4A shows that the antibody titers obtained after immunization with poly-GA constructs linked to a Th epitope (SEQ ID NOs: 68, 69, 70, 80, and 88) were highly immunogenic and produced high antibody titers; whereas antibody titers obtained after immunization with poly-GA peptides not linked to a Th epitope (GA)₅ or (GA)₁₀ and (GA)₁₅ (SEQ ID NOs: 1 and 2) were not immunogenic.

The antibody titers obtained after immunization with poly-GP constructs linked to a Th epitope (SEQ ID NOs: 98, 99, and 110) and poly-GP constructs not linked to a Th epitope (GP)₁₀ and (GP)₁₅ (SEQ ID NOs: 4 and 5) were analyzed by ELISA against the (GP)₁₅-KKK-εK-UBITh®1 construct of SEQ ID NO: 99. FIG. 4B shows that the antibody titers obtained after immunization with poly-GP constructs linked to a Th epitope (SEQ ID NOs: 98, 99, and 110) were highly immunogenic and produced high antibody titers; whereas antibody titers obtained after immunization with poly-GP peptides not linked to a Th epitope (GP)₁₀ and (GP)₁₅ (SEQ ID NOs: 4 and 5) were not immunogenic.

The antibody titers obtained after immunization with poly-GR constructs linked to a Th epitope (SEQ ID NOs: 130 and 148) and poly-GR constructs not linked to a Th epitope (GR)₁₀, (GR)₁₅, and (GR)₂₅ (SEQ ID NOs: 7, 8, and 9) were analyzed by ELISA against the (GR)₂₅-KKK-εK-UBITh®1 construct of SEQ ID NO: 130. FIG. 4C shows that the antibody titers obtained after immunization with poly-GR constructs linked to a Th epitope (SEQ ID NOs: 130 and 148) were highly immunogenic and produced high antibody titers; whereas antibody titers obtained after immunization with poly-GR peptides not linked to a Th epitope (GR)₁₀, (GR)₁₅, and (GR)₂₅ (SEQ ID NOs: 7, 8, and 9) were not immunogenic.

The antibody titers obtained after immunization with poly-PR constructs linked to a Th epitope (SEQ ID NOs: 161 and 173) and poly-PR constructs not linked to a Th epitope (PR)₁₀ (SEQ ID NO: 10) were analyzed by ELISA against the (PR)₁₀ construct of SEQ ID NO: 10. FIG. 4D shows that the antibody titers obtained after immunization with poly-PR constructs linked to a Th epitope (SEQ ID NOs: 161 and 173) were highly immunogenic and produced high antibody titers; whereas antibody titers obtained after immunization with poly-PR peptide not linked to a Th epitope (PR)₁₀ (SEQ ID NO: 10) was slightly immunogenic above the background level.

It is therefore of great interest and industrial application in that these structurally simple yet conformationally diverse DPR, nonimmunogenic on their own in most of the cases, can be rendered immunogenic to elicit high titer antibodies to the corresponding target B cell epitope peptides, through immunogen designs employing various UBITh® Th peptides by special linkage to the target “B” epitopes, regardless of the lengths of the repeats. Such DPR immunogens, when formulated into vaccines, can be applied for intervention to treat patients with certain neurodegenerative diseases, due to the presence in the brain of such DPRs, for immunotherapy of these diseases.

Such vaccine formulation efficacy can be assessed in a mouse model, such as the one described in Liu, Y, et al., “C9orf72 BAC Mouse Model with Motor Deficits and Neurodegenerative Features of ALS/FTD”, Neuron, 90, 521-534 (2016). For example, such a mouse model could be immunized with one or more of the DPR peptide immunogen constructs described herein to demonstrate that the immunized animals produce anti-DPR antibodies (e.g., anti-GA, anti-GP, etc.), depending on the immunogen. The immunized animals could be further analyzed to determine if the antibodies can be detected in both serum and brain lysates. Then such immunized animals could be analyzed to determine if the anti-DPR specific antibodies co-localize with their respective poly-DPR protein aggregates. These vaccine formulations containing these DPR immunogen constructs could then be tested in cohorts of age-matched, repeat length matched, subjects.

TABLE 1
Amino Acid Sequences of Dipeptide Repeat Protein (DRP) of C9orf72

| SEQ ID | Peptide | |
|---|---|---|
| NO | Description | Sequence |
| 1 | (GA)₁₀ | GAGAGAGAGAGAGAGAGAGA |
| 2 | (GA)₁₅ | GAGAGAGAGAGAGAGAGAGAGAGAGAGAGA |
| 3 | (GA)₂₅ | GAGAGAGAGAGAGAGAGAGAGAGAGAGAGAGAGAGAGAGAGAGAGAGAGA |
| 4 | (GP)₁₀ | GPGPGPGPGPGPGPGPGPGP |
| 5 | (GP)₁₅ | GPGPGPGPGPGPGPGPGPGPGPGPGPGPGP |
| 6 | (GP)₂₅ | GPGPGPGPGPGPGPGPGPGPGPGPGPGPGPGPGPGPGPGPGPGPGPGPGP |
| 7 | (GR)₁₀ | GRGRGRGRGRGRGRGRGRGR |
| 8 | (GR)₁₅ | GRGRGRGRGRGRGRGRGRGRGRGRGRGRGR |
| 9 | (GR)₂₅ | GRGRGRGRGRGRGRGRGRGRGRGRGRGRGRGRGRGRGRGRGRGRGRGRGR |
| 10 | (PR)₁₀ | PRPRPRPRPRPRPRPRPRPR |
| 11 | (PR)₁₅ | PRPRPRPRPRPRPRPRPRPRPRPRPRPRPR |
| 12 | (PR)₂₅ | PRPRPRPRPRPRPRPRPRPRPRPRPRPRPRPRPRPRPRPRPRPRPRPRPR |
| 13 | (PA)₁₀ | PAPAPAPAPAPAPAPAPAPA |
| 14 | (PA)₁₅ | PAPAPAPAPAPAPAPAPAPAPAPAPAPAPA |
| 15 | (PA)₂₅ | PAPAPAPAPAPAPAPAPAPAPAPAPAPAPAPAPAPAPAPAPAPAPAPAPA |

TABLE 2
Amino Acid Sequences of Pathogen Protein Derived Th Epitopes Including
Idealized Artificial Th Epitopes for Employment in the Design of Peptide
Immunogen Constructs

| Description | Sequence | SEQ ID NO |
|---|---|---|
| MvF Th | DLSDLKGLLLHKLDGL | 16 |
| (SSAL1 Th1) | EI EIR III RIE I | 17 |
| | V V VVV V V | 18 |
| | F F FFF F F | 19 |
| | XXSXXXGXXXHXXXGX | 20 |
| MvF1 Th (UBITh ®5) | LSEIKGVIVHRLEGV | 21 |
| MvF2 Th | ISEIKGVIVHKIEGI | 22 |
| MvF3 Th | ISISEIKGVIVHKIEGILF | 23 |
| | T RT TR T | 24 |
| | ISIXEIXXVIVXXIEXILF | 25 |
| | KKKISISEIKGVIVHKIEGILF | 26 |
| KKKMvF3 Th | T RT TR T | 27 |
| | KKKISIXEIXXVIVXXIEXILF | 28 |
| MvF4 Th (UBITh ®3) | ISISEIKGVIVHKIETILF | 29 |
| | T RT TR | 30 |
| | ISIXEIXXVIVXXIETILF | 31 |
| MvF5 Th (UBITh ®1) | ISITEIKGVIVHRIETILF | 32 |
| KKKMvF5 Th (UBITh ®1a) | KKKISITEIKGVIVHRIETILF | 33 |
| HBsAg1 Th | KKKLFLLTKLLTLPQSLD | 34 |
| (SSAL2 Th2) | RRRIKII RII I L IR | 35 |
| | VRVV VV V I V | 36 |
| | F FF FF F V F | 37 |
| | F | 38 |
| | XXXXXXXTXXXTXPXSXX | 39 |
| HBsAg2 Th | KKKIITITRIITIPQSLD | 40 |
| | FFLL L ITTI | 41 |
| | KKKXXXXTRIXTIXXXXD | 42 |
| HBsAg3 Th (UBITh ®2) | KKKIITITRIITIITTID | 43 |
| HBsAg Th (UBITh ®4) | FFLLTRILTIPQSLD | 44 |
| KKK-HBsAg Th | KKKFFLLTRILTIPQSLD | 45 |
| HBsAg Th | FFLLTRILTIPQSL | 46 |
| Bordetella pertussis Th (UBITh ®7) | GAYARCPNGTRALTVAELRGNAEL | 47 |
| Cholera Toxin Th | ALNIWDRFDVFCTLGATTGYLKGNS | 48 |
| Clostridium tetani TT1 Th | QYIKANSKFIGITEL | 49 |
| Clostridium tetani1 Th (UBITh ®6) | KKQYIKANSKFIGITEL | 50 |
| Clostridium tetani TT2 Th | FNNFTVSFWLRVPKVSASHLE | 51 |
| Clostridium tetani TT3 Th | KFIIKRYTPNNEIDSF | 52 |
| Clostridium tetani TT4 Th | VSIDKFRIFCKALNPK | 53 |
| Clostridium tetani2 Th | WVRDIIDDFTNESSQKT | 54 |
| Diphtheria Th | DSETADNLEKTVAALSILPGHGC | 55 |
| EBV BHRF1 Th | AGLTLSLLVICSYLFISRG | 56 |
| EBV EBNA-1 Th | PGPLRESIVCYFMVFLQTHI | 57 |
| EBV CP Th | VPGLYSPCRAFFNKEELL | 58 |
| EBV GP340 Th | TGHGARTSTEPTTDY | 59 |
| EBV BPLF1 Th | KELKRQYEKKLRQ | 60 |
| EBV EBNA-2 | TVFYNIPPMPL | 61 |
| HCMV IE1 Th | DKREMWMACIKELH | 62 |
| Influenza MP1_1 Th | FVFTLTVPSER | 63 |
| Influenza MP1_2 Th | SGPLKAEIAQRLEDV | 64 |
| Influenza NSP1 Th | DRLRRDQKS | 65 |
| Plasmodium falciparum Th | DHEKKHAKMEKASSVFNVVNS | 66 |
| Schistosoma mansoni Th | KWFKTNAPNGVDEKHRH | 67 |

TABLE 10
ELISA

| | | | (SEQ ID NO: 70) | | | | | | (SEQ ID NO: 69) | | | | | |
|---|---|---|---|---|---|---|---|---|---|---|---|---|---|---|
| | | | (GA)₂₅-KKK-εK-UBITh ®1 Titers | | | | | | (GA)₁₅-KKK-εK-UBITh ®1 Titers | | | | | |
| Grp | | GP | 0 | 3 | 6 | 9 | 12 | 15 | 0 | 3 | 6 | 9 | 12 | 15 |
| # | Immunogen | # | wpi | wpi | wpi | wpi | wpi | wpi | wpi | wpi | wpi | wpi | wpi | wpi |
| 1 | (GA)₁₀-KKK-εK- | 6186 | 0.2 | 3.05 | 5.15 | 6.52 | 7.29 | 8.32 | 0.18 | 5.67 | >10 | >10 | >10 | >10 |
| | UBITh ®1 | 6187 | 0.08 | 4.82 | >10 | >10 | >10 | 9.83 | 0.12 | >10 | >10 | >10 | >10 | >10 |
| | SEQ ID NO: 68 | 6186 | 0.08 | 3.95 | 6.43 | 7.64 | 7.39 | 7.27 | 0.13 | 10.2 | >10 | >10 | >10 | >10 |
| 2 | (GA)₁₅-KKK-εK- | 6189 | 0.08 | 4.87 | >10 | >10 | >10 | 7.21 | 0.12 | >10 | >10 | >10 | >16 | 8.84 |
| | UBITh ®1 | 6190 | 0.08 | 4.87 | >10 | >10 | >10 | 9.05 | 0.12 | 7.94 | >10 | >10 | 9.29 | 8.39 |
| | SEQ ID NO: 69 | 6191 | 0.08 | 4.99 | >10 | >10 | >10 | 8.59 | 0.14 | 10.76 | >10 | >10 | >10 | 10.01 |
| 3 | UBITh ®1-εK- | 6192 | 0.09 | 4.65 | >10 | >10 | >10 | >10 | 0.1 | >10 | >10 | >10 | >10 | >10 |
| | KKK-(GA)₁₀-KKK- | 6193 | 0.11 | 4.01 | >10 | >10 | >10 | >10 | 0.14 | 8.43 | >10 | >10 | >10 | >10 |
| | εK-UBITh ®1 | 6194 | 0.19 | 5.21 | >10 | 7.89 | 8.73 | 10.87 | 0.16 | >10 | >10 | 9.37 | 7.66 | 9.48 |
| | SEQ ID NO: 80 | | | | | | | | | | | | | |
| 4 | (GA)₂₅-KKK-εK- | 6195 | 0.08 | 3.49 | 7.18 | 8.8 | >10 | >10 | 0.11 | 5.28 | >10 | >10 | >10 | >10 |
| | UBITh ®1 | 6196 | 0.07 | 4.84 | 7.62 | 7.75 | 9.31 | >10 | 0.13 | 5.71 | 9.85 | 7.69 | 9.32 | >10 |
| | SEQ ID NO: 70 | 6197 | 0.07 | 4.14 | >10 | >10 | >10 | >10 | 0.1 | 5.73 | >10 | >10 | >10 | >10 |
| 5 | UBITh ®3-εK- | 6198 | 0.08 | 4.49 | >10 | >10 | 9.28 | 9.12 | 0.09 | 9.8 | >10 | >10 | 9.83 | 10.12 |
| | KKK-(GA)₂₅-KKK- | 6199 | 0.07 | 4.79 | >10 | >10 | 10.95 | 10 | 0.12 | >10 | >10 | >10 | 11.07 | 11.05 |
| | εK-UBITh ®1 | 6200 | 0.1 | 3.69 | 9.96 | 10.85 | 8.58 | 9.2 | 0.1 | 6.52 | >10 | >10 | 9.65 | 11.29 |
| | SEQ ID NO: 88 | | | | | | | | | | | | | |
| 6 | (GP)₁₀-KKK-εK- | 6201 | 0.1 | 0 | 3.38 | 3.21 | 3.11 | 4.22 | 0.12 | 2.96 | 4.76 | 4.12 | 3.73 | 4.73 |
| | UBITh ®1 | 6202 | 0.19 | 0 | 3.3 | 3.27 | 3.16 | 3.23 | 0.14 | 2.63 | 4.59 | 4.28 | 3.51 | 3.6 |
| | SEQ ID NO: 98 | 6203 | 0.08 | 0 | 0.24 | 2.79 | 2.81 | 4.18 | 0.13 | 0 | 3.23 | 3.56 | 3.3 | 4.69 |
| 7 | (GP)₁₅-KKK-εK- | 6204 | 0.08 | 0 | 2.95 | 3.2 | 3.3 | 3.21 | 0.12 | 2.38 | 3.91 | 4.5 | 4.38 | 3.87 |
| | UBITh ®1 | 6205 | 0.08 | 0 | 2.82 | 2.62 | 2.41 | 3.07 | 0.13 | 3.47 | 4.32 | 3.65 | 3.39 | 3.54 |
| | SEQ ID NO: 99 | 6206 | 0.07 | 0 | 3.18 | 3.95 | 4.06 | 4.14 | 0.11 | 3.25 | 3.63 | 4.89 | 4.24 | 4.64 |
| 8 | UBITh ®1-εK- | 6207 | 0.07 | 0 | 2.08 | 2.9 | 2.92 | 3.9 | 0.14 | 2.67 | 3.3 | 3.47 | 3.43 | 4.76 |
| | KKK-(GP)₁₀-KKK- | 6208 | 0.1 | 2.26 | 4.71 | 4.81 | 4.5 | 4.19 | 0.11 | 3.19 | 4.96 | 4.75 | 4.58 | 4.49 |
| | εK-UBITh ®1 | 6209 | 0.11 | 0 | 3.4 | 3.36 | 3.61 | 3.49 | 0.14 | 3.56 | 4.84 | 4.57 | 4.15 | 4.19 |
| | SEQ ID NO: 110 | | | | | | | | | | | | | |
| 9 | (GR)₂₅-KKK-εK- | 6210 | 0.19 | 0 | 4.46 | 4.94 | 4.92 | 4.86 | 0.15 | 3.11 | 5.09 | 5.22 | 5.2 | 5.11 |
| | UBITh ®1 | 6211 | 0.08 | 0 | 3.64 | 4.57 | 4.49 | 4.21 | 0.12 | 3.85 | 4.81 | 5.04 | 4.95 | 4.8 |
| | SEQ ID NO: 130 | 6212 | 0.08 | 0 | 4.52 | 4.34 | 3.9 | 3.61 | 0.11 | 3.38 | 4.98 | 4.86 | 4.66 | 4.6 |
| 10 | UBITh ®3-εK- | 6213 | 0.07 | 1.18 | 5 | 4.96 | 4.85 | 4.8 | 0.12 | 4.68 | 5.59 | 5.37 | 5.33 | 5.16 |
| | KKK-(GR)₂₅-KKK- | 6214 | 0.08 | 3.18 | 4.75 | 4.84 | 4.86 | 4.68 | 0.11 | 4.7 | 5.09 | 5.52 | 4.96 | 4.89 |
| | εK-UBITh ®1 | 6215 | 0.08 | 2.02 | 5.81 | 4.93 | 4.91 | 4.85 | 0.11 | 4.56 | 5.36 | 5.37 | 5.2 | 5.05 |
| | SEQ ID NO: 148 | | | | | | | | | | | | | |

| | | | (SEQ ID NO. 88) | | | | | | (SEQ ID NO: 68) | | | | | |
|---|---|---|---|---|---|---|---|---|---|---|---|---|---|---|
| | | | UBITh ®3-εK-KKK-(GA)₂₅-KKK-εK-UBITh ®1 Titers | | | | | | (GA)₁₀-KKK-εK-UBITh ®1 Titers | | | | | |
| Grp | | GP | 0 | 3 | 6 | 9 | 12 | 15 | 0 | 3 | 6 | 9 | 12 | 15 |
| # | Immunogen | # | wpi | wpi | wpi | wpi | wpi | wpi | wpi | wpi | wpi | wpi | wpi | wpi |
| 1 | (GA)₁₀-KKK-εK- | 6186 | 0.25 | 5.28 | 5.59 | 5.58 | 6.01 | 6.1 | 0.34 | 5.53 | >10 | 12.81 | 12.26 | 11.88 |
| | UBITh ®1 | 6187 | 0.19 | >10 | >10 | >10 | >10 | 8.28 | 0.17 | 9.52 | >10 | >10 | >10 | >10 |
| | SEQ ID NO: 68 | 6188 | 0.17 | 7.21 | 6.76 | 6.91 | 5.97 | 6.07 | 0.15 | 8.48 | >10 | >10 | >10 | 11.05 |
| 2 | (GA)₁₅-KKK-εK- | 6189 | 0.18 | 7.32 | >10 | 9.86 | 7.92 | 6.63 | 0.14 | 13.65 | >10 | >10 | >10 | 7.77 |
| | UBITh ®1 | 6190 | 0.17 | 7.95 | >10 | >10 | >10 | 9.45 | 0.12 | 7.12 | >10 | >10 | 8.87 | 8.69 |
| | SEQ ID NO: 69 | 6191 | 0.2 | 10.16 | >10 | >10 | >10 | 10.04 | 0.13 | 8.65 | >10 | >10 | >10 | 9.5 |
| 3 | UBITh ®1-εK- | 6192 | 0.19 | 11.51 | >10 | >10 | >10 | >10 | 0.12 | 9.16 | >10 | >10 | >10 | >10 |
| | KKK-(GA)₁₀-KKK- | 6193 | 0.25 | 7.64 | >10 | >10 | >10 | >10 | 0.15 | 6.59 | >10 | >10 | >10 | >10 |
| | εK-UBITh ®1 | 6194 | 0.25 | >10 | >10 | 9.95 | 10.88 | >10 | 0.3 | >10 | >10 | 8.58 | 8.05 | 10.5 |
| | SEQ ID NO: 80 | | | | | | | | | | | | | |
| 4 | (GA)₂₅-KKK-εK- | 6195 | 0.2 | 5.22 | >10 | >10 | >10 | >10 | 0.2 | 5.08 | 6.89 | 8.36 | 8.63 | >10 |
| | UBITh ®1 | 6196 | 0.16 | 5.61 | 8.92 | 10.49 | >10 | >10 | 0.18 | 5.27 | 7.45 | 7.08 | 7.28 | >10 |
| | SEQ ID NO: 70 | 6197 | 0.2 | 5.59 | >10 | >10 | >10 | >10 | 0.16 | 5.35 | >10 | >10 | >10 | >10 |
| 5 | UBITh ®3-εK- | 6198 | 0.17 | 9.24 | >10 | >10 | >10 | >10 | 0.19 | 7.4 | >10 | >10 | >10 | 10.8 |
| | KKK-(GA)₂₅-KKK- | 6199 | 0.19 | >10 | >10 | >10 | >10 | >10 | 0.16 | 8.7 | >10 | >10 | >10 | 11.4 |
| | εK-UBITh ®1 | 6200 | 0.2 | 6.56 | >19 | >10 | >10 | >10 | 0.18 | 5.76 | >10 | >10 | 9.75 | 11.44 |
| | SEQ ID NO: 88 | | | | | | | | | | | | | |
| 6 | (GP)₁₀-KKK-εK- | 6201 | 0.3 | 2.94 | 4.63 | 4.17 | 3.9 | 4.61 | 0.17 | 2.77 | 4.59 | 4.32 | 3.64 | 4.47 |
| | UBITh ®1 | 6202 | 0.19 | 2.46 | 4.62 | 4.32 | 3.75 | 3.82 | 0.16 | 2.23 | 4.18 | 3.82 | 3.37 | 3.47 |
| | SEQ ID NO: 98 | 6203 | 0.18 | 5.84 | 3.17 | 3.5 | 3.29 | 4.63 | 0.17 | 0 | 3.22 | 3.43 | 3.2 | 4.56 |
| 7 | (GP)₁₅-KKK-εK- | 6204 | 0.18 | 2.2 | 3.91 | 4.47 | 4.53 | 3.98 | 0.18 | 1.99 | 3.71 | 4.65 | 4.23 | 3.74 |
| | UBITh ®1 | 6205 | 0.16 | 2.81 | 3.26 | 3.21 | 3.02 | 3.32 | 0.15 | 3.47 | 4.42 | 3.77 | 3.38 | 3.55 |
| | SEQ ID NO: 99 | 6206 | 0.13 | 3.46 | 3.68 | 4.86 | 4.55 | 4.68 | 0.13 | 3.26 | 3.55 | 4.23 | 4.2 | 4.51 |
| 8 | UBITh ®1-εK- | 6207 | 0.19 | 2.4 | 3.2 | 3.56 | 3.45 | 4.56 | 0.17 | 1.62 | 3.17 | 3.38 | 3.25 | 4.36 |
| | KKK-(GP)₁₀-KKK- | 6208 | 0.18 | 3.15 | 4.91 | 5.07 | 4.67 | 4.53 | 0.11 | 2.99 | 4.89 | 4.63 | 4.51 | 4.47 |
| | εK-UBITh ®1 | 6209 | 0.22 | 3.21 | 4.2 | 4.19 | 4.49 | 4.53 | 0.17 | 3.21 | 4.42 | 4.8 | 4.06 | 4.1 |
| | SEQ ID NO: 110 | | | | | | | | | | | | | |
| 9 | (GR)₂₅-KKK-εK- | 6210 | 0.19 | 2.92 | 5.08 | 5.05 | 5.1 | 5.06 | 0.19 | 2.9 | 5.09 | 5.28 | 5.18 | 5.15 |
| | UBITh ®1 | 6211 | 0.16 | 3.64 | 4.8 | 4.98 | 4.86 | 4.81 | 0.14 | 3.54 | 4.8 | 4.96 | 4.87 | 4.74 |
| | SEQ ID NO: 130 | 6212 | 0.13 | 3.3 | 4.87 | 4.7 | 4.61 | 4.46 | 0.14 | 3.18 | 4.99 | 4.8 | 4.65 | 4.56 |
| 10 | UBITh ®3-εK- | 6213 | 0.15 | 4.57 | 5.39 | 5.21 | 5.08 | 5.01 | 0.12 | 4.65 | 5.52 | 5.34 | 5.34 | 5.2 |
| | KKK-(GR)₂₅-KKK- | 6214 | 0.13 | 4.67 | 5.06 | 5 | 5.01 | 4.98 | 0.11 | 4.68 | 5.03 | 5.25 | 4.92 | 4.92 |
| | εK-UBITh ®1 | 6215 | 0.15 | 4.5 | 6.94 | 5.15 | 5.12 | 5.07 | 0.14 | 4.33 | 5.44 | 5.4 | 5.12 | 5.01 |
| | SEQ ID NO: 148 | | | | | | | | | | | | | |

| | | | (SEQ ID NO: 99) | | | | | | (SEQ ID NO: 98) | | | | | |
|---|---|---|---|---|---|---|---|---|---|---|---|---|---|---|
| | | | (GP)₁₅-KKK-εK-UBITh ®1 Titers | | | | | | (GP)₁₀-KKK-εK-UBITh ®1 Titers | | | | | |
| Grp | | GP | 0 | 3 | 6 | 9 | 12 | 15 | 0 | 3 | 6 | 9 | 12 | 15 |
| # | Immunogen | # | wpi | wpi | wpi | wpi | wpi | wpi | wpi | wpi | wpi | wpi | wpi | wpi |
| 1 | (GA)₁₀-KKK-εK- | 6186 | 0.28 | 2.23 | 3.32 | 3.93 | 3.57 | 4.6 | 0.12 | 3.87 | 3.23 | 4.07 | 3.45 | 4.1 |
| | UBITh ®1 | 6187 | 0.11 | 3.11 | 4.88 | 9.67 | 10.2 | 9.58 | 0.11 | 3.92 | 4.8 | 7.03 | 8.14 | 7.48 |
| | SEQ ID NO: 68 | 6188 | 0.1 | 1.52 | 3 | 3.55 | 3.51 | 3.52 | 0.09 | 2.77 | 2.81 | 3.53 | 3.5 | 3.51 |
| 2 | (GA)₁₅-KKK-εK- | 6189 | 0.1 | 3.18 | 3.67 | 4.85 | 4.11 | 4.89 | 0.1 | 3.09 | 3.29 | 4.83 | 4.01 | 4.69 |
| | UBITh ®1 | 6190 | 0.09 | 3.07 | 4.13 | 4.75 | 4.62 | 4.56 | 0.09 | 3.27 | 4.1 | 4.82 | 4.75 | 4.58 |
| | SEQ ID NO: 69 | 6191 | 0.09 | 3.48 | 5 | 5.08 | 5.34 | 5.14 | 0.1 | 4.49 | 4.82 | 4.96 | 5.2 | 4.98 |
| 3 | UBITh ®1-εK- | 6192 | 0.11 | 1.35 | 3.23 | 3.4 | 4.73 | 4.71 | 0.12 | 1.96 | 3.23 | 3.33 | 4.87 | 4.71 |
| | KKK-(GA)₁₀-KKK- | 6193 | 0.12 | 1.79 | 4.77 | 4.32 | 4.73 | 4.72 | 0.1 | 5.46 | 1.95 | 3.49 | 3.63 | 4.13 |
| | εK-UBITh ®1 | 6194 | 0.24 | 3.13 | 3.69 | 3.56 | 4.63 | 4.73 | 0.16 | 3.43 | 3.84 | 3.66 | 4.66 | 4.68 |
| | SEQ ID NO: 80 | | | | | | | | | | | | | |
| 4 | (GA)₂₅-KKK-εK- | 6195 | 0.09 | 3.78 | 5.73 | 5.03 | 5.44 | 5.15 | 0.12 | 0 | 5 | 5.2 | 5.01 | 5 |
| | UBITh ®1 | 6196 | 0.08 | 4.84 | 5.67 | 5.26 | 5.37 | 5.49 | 0.1 | 4.9 | 5.29 | 5.32 | 5.33 | 5.26 |
| | SEQ ID NO: 70 | 6197 | 0.08 | 3.98 | 5.98 | 5.5 | 5.93 | 6.23 | 0.11 | 3.88 | 5.32 | 5.79 | 5.58 | 5.72 |
| 5 | UBITh ®3-εK- | 6198 | 0.07 | 1.19 | 3.43 | 4.72 | 4.83 | 4.88 | 0.09 | 3.03 | 3.23 | 4.97 | 4.54 | 4.74 |
| | KKK-(GA)₂₅-KKK- | 6199 | 0.07 | 3.09 | 4.52 | 4.89 | 4.89 | 5.02 | 0.11 | 3.58 | 4.3 | 5.24 | 4.66 | 4.87 |
| | εK-UBITh ®1 | 6200 | 0.1 | 1.56 | 3.35 | 3.86 | 3.85 | 4.7 | 0.12 | 2.19 | 2.52 | 3.42 | 3.32 | 4.29 |
| | SEQ ID NO: 88 | | | | | | | | | | | | | |
| 6 | (GP)₁₀-KKK-εK- | 6201 | 0.1 | 8.66 | >10 | >10 | >10 | >10 | 0.19 | 10.51 | >10 | >10 | >10 | >10 |
| | UBITh ®1 | 6202 | 0.22 | 11.6 | >10 | >10 | >10 | >10 | 0.13 | 13.5 | >10 | >10 | >10 | >10 |
| | SEQ ID NO: 98 | 6203 | 0.09 | 6.75 | 8.9 | 8.84 | 8.25 | 8.35 | 0.14 | 9.49 | 8.8 | 8.37 | 7.64 | 7.98 |
| 7 | (GP)₁₅-KKK-εK- | 6204 | 0.09 | 10.6 | >10 | >10 | >10 | 12.49 | 0.11 | >10 | >10 | 10.23 | 11.58 | 12.33 |
| | UBITh ®1 | 6205 | 0.08 | 8.9 | >10 | 8.74 | 9.9 | 9.97 | 0.1 | 10.66 | >10 | 9.13 | 9.35 | 9.56 |
| | SEQ ID NO: 99 | 6206 | 0.08 | >10 | 9.78 | 8.89 | 10.5 | 9.99 | 0.09 | >10 | 9.21 | 9.57 | 9.33 | 9.15 |
| 8 | UBITh ®1-εK- | 6207 | 0.07 | 9.95 | >10 | 10.06 | 9.62 | 8.73 | 0.14 | >10 | >10 | 11.7 | 9.52 | 8.9 |
| | KKK-(GP)₁₀-KKK- | 6208 | 0.1 | >10 | >10 | 11 | 10.01 | 8.8 | 0.11 | >10 | >10 | 11.99 | 10.16 | 8.76 |
| | εK-UBITh ®1 | 6209 | 0.09 | >10 | >10 | 10.8 | >10 | >10 | 0.11 | >10 | >10 | >10 | >10 | >10 |
| | SEQ ID NO: 110 | | | | | | | | | | | | | |
| 9 | (GR)₂₅-KKK-εK- | 6210 | 0.2 | 2.7 | 3.76 | 4.68 | 4.87 | 4.87 | 0.12 | 2.08 | 3.68 | 3.48 | 4.8 | 4.88 |
| | UBITh ®1 | 6211 | 0.08 | 3.3 | 3.62 | 4.57 | 4.75 | 4.79 | 0.15 | 3.26 | 3.42 | 4.27 | 4.61 | 4.88 |
| | SEQ ID NO: 130 | 6212 | 0.07 | 3.28 | 4.03 | 4.27 | 4.17 | 4.68 | 0.11 | 3.24 | 3.71 | 4.07 | 4.03 | 4.66 |
| 10 | UBITh ®3-εK- | 6213 | 0.08 | 2.43 | 3.28 | 4.25 | 5.08 | 4.74 | 0.11 | 2.27 | 3.28 | 3.88 | 4.37 | 4.69 |
| | KKK-(GR)₂₅-KKK- | 6214 | 0.08 | 3.61 | 4.46 | 4.44 | 4.11 | 4.48 | 0.11 | 3.57 | 4.13 | 4.23 | 4.02 | 4.71 |
| | εK-UBITh ®1 | 6215 | 0.09 | 3.31 | 4.32 | 6 | 5.05 | 4.96 | 0.12 | 3.13 | 3.71 | 4.86 | 4.9 | 4.81 |
| | SEQ ID NO: 148 | | | | | | | | | | | | | |

| | | | (SEQ ID NO: 148) | | | | | | (SEQ ID NO: 130) | | | | | |
|---|---|---|---|---|---|---|---|---|---|---|---|---|---|---|
| | | | UBITh ®3-εK-KKK-(GR)₂₅-KKK-εK-UBITh ®1 Titers | | | | | | (GR)₂₅-KKK-εK-UBITh ®1 Titers | | | | | |
| Grp | | GP | 0 | 3 | 6 | 9 | 12 | 15 | 0 | 3 | 6 | 9 | 12 | 15 |
| # | Immunogen | # | wpi | wpi | wpi | wpi | wpi | wpi | wpi | wpi | wpi | wpi | wpi | wpi |
| 1 | (GA)₁₀-KKK-εK- | 6186 | 0.16 | 0 | 2.79 | 3.11 | 3.44 | 4.12 | 0.18 | 0 | 3.01 | 3.26 | 3.77 | 4.37 |
| | UBITh ®1 | 6187 | 0.11 | 2.99 | 4.65 | 4.93 | 4.97 | 4.94 | 0.15 | 3.85 | 4.75 | 4.9 | 4.97 | 4.89 |
| | SEQ ID NO: 68 | 6188 | 0.1 | 0 | 1.89 | 3.09 | 3.33 | 3.33 | 0.14 | 0 | 3.09 | 3.4 | 3.59 | 3.54 |
| 2 | (GA)₁₅-KKK-εK- | 6189 | 0.1 | 3.67 | 4.81 | 4.97 | 4.93 | 5.04 | 0.15 | 3.51 | 4.77 | 4.85 | 4.86 | 4.97 |
| | UBITh ®1 | 6190 | 0.09 | 3 | 3.68 | 3.89 | 4.34 | 4.54 | 0.15 | 2.71 | 3.39 | 3.58 | 4 | 4.23 |
| | SEQ ID NO: 69 | 6191 | 0.1 | 4.26 | 5.06 | 5.3 | 5.52 | 5.28 | 0.16 | 4.18 | 5.01 | 5.14 | 5.34 | 5.16 |
| 3 | UBITh ®1-εK- | 6192 | 0.15 | 0 | 3.06 | 3.91 | 4.78 | 4.71 | 0.16 | 1.5 | 3.01 | 3.24 | 4.28 | 4.05 |
| | KKK-(GA)₁₀-KKK- | 6193 | 0.14 | 0 | 0.3 | 2.76 | 3.14 | 4.09 | 0.18 | 0 | 0.54 | 2.6 | 3.1 | 3.65 |
| | εK-UBITh ®1 | 6194 | 0.2 | 3.1 | 4.18 | 4.13 | 4.77 | 5.04 | 0.18 | 3.36 | 4.14 | 3.56 | 4.71 | 4.86 |
| | SEQ ID NO: 80 | | | | | | | | | | | | | |
| 4 | (GA)₂₅-KKK-εK- | 6195 | 0.11 | 3.59 | 5.36 | 5.75 | 6.25 | 6.12 | 0.14 | 4.04 | 5.15 | 5.29 | 5.56 | 5.41 |
| | UBITh ®1 | 6196 | 0.1 | 4.37 | 5.7 | 5.75 | 8.96 | 10.76 | 0.15 | 3.88 | 5.26 | 5.29 | 6.36 | 7.63 |
| | SEQ ID NO: 70 | 6197 | 0.1 | 3.6 | 6.31 | 6.25 | 7.57 | 7.72 | 0.15 | 3.4 | 5.41 | 5.53 | 6.11 | 6.27 |
| 5 | UBITh ®3-εK- | 6198 | 0.11 | 2.19 | 3.35 | 4.74 | 4.82 | 4.92 | 0.13 | 1.76 | 3.27 | 4.61 | 4.74 | 4.79 |
| | KKK-(GA)₂₅-KKK- | 6199 | 0.1 | 2.94 | 4.53 | 4.82 | 4.95 | 5.08 | 0.15 | 3.24 | 3.88 | 4.57 | 4.8 | 4.94 |
| | εK-UBITh ®1 | 6200 | 0.13 | 1.79 | 4.54 | 4.7 | 4.62 | 4.95 | 0.16 | 1.42 | 3.84 | 3.78 | 3.68 | 4.25 |
| | SEQ ID NO: 88 | | | | | | | | | | | | | |
| 6 | (GP)₁₀-KKK-εK- | 6201 | 0.12 | 0 | 0.31 | 0 | 0 | 3.21 | 0.2 | 0 | 0.76 | 0 | 0 | 3.1 |
| | UBITh ®1 | 6202 | 0.17 | 0 | 0 | 1.27 | 2.76 | 2.9 | 0.2 | 0 | 0 | 1.9 | 3.03 | 3 |
| | SEQ ID NO: 98 | 6203 | 0.12 | 0 | 8.83 | 0 | 0 | 2.95 | 0.14 | 0 | 0 | 0 | 0 | 2.63 |
| 7 | (GP)₁₅-KKK-εK- | 6204 | 0.11 | 0 | 0 | 0.99 | 3.08 | 3.14 | 0.14 | 0 | 0 | 0.5 | 3.07 | 3.12 |
| | UBITh ®1 | 6205 | 0.1 | 0 | 0 | 0 | 0 | 2.84 | 0.14 | 0 | 0 | 0 | 0 | 2.63 |
| | SEQ ID NO: 99 | 6206 | 0.1 | 0 | 0 | 1.9 | 2.68 | 3.02 | 0.14 | 0 | 0 | 1.99 | 2.75 | 3.04 |
| 8 | UBITh ®1-εK- | 6207 | 0.1 | 0 | 0 | 0 | 1.48 | 2.81 | 0.15 | 0 | 0 | 0 | 1.76 | 2.53 |
| | KKK-(GP)₁₀-KKK- | 6208 | 0.17 | 0 | 0 | 0.2 | 3.1 | 2.97 | 0.15 | 0 | 0 | 0 | 3.04 | 2.85 |
| | εK-UBITh ®1 | 6209 | 0.13 | 0 | 0 | 3.2 | 4.19 | 4.21 | 0.19 | 0.77 | 2.81 | 3.28 | 4.21 | 3.97 |
| | SEQ ID NO: 110 | | | | | | | | | | | | | |
| 9 | (GR)₂₅-KKK-εK- | 6210 | 0.18 | 5.11 | 7.42 | 6.7 | 6.85 | 6.2 | 0.2 | 5.19 | 7.29 | 6.19 | 6.67 | 6.13 |
| | UBITh ®1 | 6211 | 0.16 | 5.75 | >10 | >10 | >10 | >10 | 0.15 | 8.27 | 10.95 | >10 | >10 | >10 |
| | SEQ ID NO: 130 | 6212 | 0.13 | 5.41 | 7.43 | 6.57 | 7.08 | 6.9 | 0.14 | 5.61 | 7.76 | 6.65 | 6.7 | 6.39 |
| 10 | UBITh ®3-εK- | 6213 | 0.13 | 4.93 | 8.7 | 7.03 | 7.42 | 6.71 | 0.14 | 5.23 | 8.4 | 7.01 | 7.16 | 6.43 |
| | KKK-(GR)₂₅-KKK- | 6214 | 0.13 | 5.59 | 9.14 | 6.23 | 7.38 | 7.76 | 0.15 | 6.62 | 8.06 | 6.12 | 6.75 | 7.09 |
| | εK-UBITh ®1 | 6215 | 0.12 | 5.36 | >10 | 7.4 | 7.45 | 6.72 | 0.17 | 6.87 | 15.08 | 7.06 | 6.8 | 6.54 |
| | SEQ ID NO: 148 | | | | | | | | | | | | | |

TABLE 11
ELISA

| | | | (SEQ ID NO: 10) | | | | | |
|---|---|---|---|---|---|---|---|---|
| (A₄₅₀ at 1/100) | | | (PR)₁₀-εK-UBITh ®1 Titers | | | | | |
| Group | | GP | 0 | 3 | 6 | 9 | 12 | 15 |
| # | Immunogen | # | wpi | wpi | wpi | wpi | wpi | wpi |
| 1 | (PR)₁₀-εK-UBITh ®1 | 6300 | 0.124 | 7.372 | 10.56 | 9.564 | >10 | >10 |
| | SEQ ID NO: 161 | 6301 | 0.134 | 6.75 | 9.043 | 7.708 | 8.651 | 7.287 |
| | | 6302 | 0.115 | 5.537 | >10 | >10 | >10 | >10 |
| 2 | UBITh ®1-εK-(PR)₁₀-εK-UBITh ®1 | 6303 | 0.121 | 5.065 | 8.057 | 7.091 | 5.692 | 5.825 |
| | SEQ ID NO: 173 | 6304 | 0.106 | 5.052 | >10 | >10 | 11.227 | >10 |
| | | 6305 | 0.077 | 5.045 | 7.125 | 8.356 | 6.723 | 6.537 |

1. A dipeptide repeat (DPR) peptide immunogen construct comprising:
a B cell epitope comprising about 10 to about 25 repeats of poly-GA, poly-GP, poly-GR, poly-PR, or poly-PA;
a heterologous T helper epitope comprising an amino acid sequence selected from the group consisting of SEQ ID NOs: 16 to 67; and
an optional heterologous spacer selected from the group consisting of an amino acid, Lys-, Gly-, Lys-Lys-Lys-, (α, ε-N)Lys, ε-N-Lys-Lys-Lys-Lys (SEQ ID NO: 221), Lys-Lys-Lys-ε-N-Lys (SEQ ID NO: 222); and
wherein the B cell epitope is covalently linked to the T helper epitope directly or through the optional heterologous spacer.

2. The DPR peptide immunogen construct of claim 1, wherein
the repeats of poly-GA have an amino acid sequence of SEQ ID NOs: 1, 2, or 3; and
the repeats of poly-GP have an amino acid sequence of SEQ ID NOs: 4, 5, or 6; and
the repeats of poly-GR have an amino acid sequence of SEQ ID NOs: 7, 8, or 9; and
the repeats of poly-PR have an amino acid sequence of SEQ ID NOs: 10, 11, or 12; and
the repeats of poly-PA have an amino acid sequence of SEQ ID NOs: 13, 14, or 15.

3. The DPR peptide immunogen construct of claim 1, wherein the amino acid sequence of the heterologous T helper epitope is selected from the group consisting of SEQ ID NO: 31, 32, and a combination thereof.

4. The DPR peptide immunogen construct of claim 1, wherein the optional heterologous spacer is (α, ε-N)Lys, ε-N-Lys-Lys-Lys-Lys (SEQ ID NO: 221), or Lys-Lys-Lys-ε-N-Lys (SEQ ID NO: 222).

5. The DPR peptide immunogen construct of claim 1 comprising the following formula:
{(Th)ₘ-(A)ₙ-(DPR)-(A)ₙ-(Th)ₘ}_{y}-X
wherein
Th is the heterologous T helper epitope;
A is the heterologous spacer;
(DPR) is a B cell epitope having repeats of poly-GA, poly-GP, poly-GR, poly-PR, or poly-PA;
X is an α-COOH or α-CONH₂ of an amino acid;
each m is from 0 to about 4;
each n is from 0 to about 10; and
y is from 1 to about 5.

6. The DPR peptide immunogen construct of claim 5, wherein
the repeats of poly-GA have an amino acid sequence of SEQ ID NOs: 1, 2, or 3; and
the repeats of poly-GP have an amino acid sequence of SEQ ID NOs: 4, 5, or 6; and
the repeats of poly-GR have an amino acid sequence of SEQ ID NOs: 7, 8, or 9; and
the repeats of poly-PR have an amino acid sequence of SEQ ID NOs: 10, 11, or 12; and
the repeats of poly-PA have an amino acid sequence of SEQ ID NOs: 13, 14, or 15.

7. The DPR peptide immunogen construct of claim 5, wherein the amino acid sequence of the heterologous T helper epitope is selected from the group consisting of SEQ ID NO: 31, 32, and a combination thereof.

8. The DPR peptide immunogen construct of claim 5, wherein the optional heterologous spacer is (α, ε-N)Lys, ε-N-Lys-Lys-Lys-Lys (SEQ ID NO: 221), or Lys-Lys-Lys-ε-N-Lys (SEQ ID NO: 222).

9. The DPR peptide immunogen construct of claim 1 comprising an amino acid sequence selected from the group consisting of SEQ ID NOs: 68 to 219, and any combination thereof.

10. The DPR peptide immunogen construct of claim 1 comprising an amino acid sequence selected from the group consisting of 68, 69, 70, 80, 88, 98, 99, 110, 130, 148, 161, 173, 218, 219, and any combination thereof.

11. A composition comprising the DPR peptide immunogen construct of claim 1.

12. A composition comprising more than one peptide immunogen construct of claim 1.

13. The composition of claim 11, wherein the DPR peptide immunogen construct has amino acid sequence selected from the group consisting of SEQ ID NOs: 68 to 219, and any combination thereof.

14. A pharmaceutical composition comprising the DPR peptide immunogen construct of claim 1 and a pharmaceutically acceptable delivery vehicle and/or adjuvant.

15. The pharmaceutical composition of claim 14, wherein
a. the DPR peptide immunogen construct is selected from the group consisting of SEQ ID NOs: 68 to 219, and any combination thereof; and
b. the adjuvant is a mineral salt of aluminum selected from the group consisting of Al(OH)₃ or AlPO₄.

16. The pharmaceutical composition of claim 14, wherein
a. the DPR peptide immunogen construct is selected from the group consisting of SEQ ID NOs: 68 to 219, and any combination thereof; and
b. the DPR peptide immunogen construct is mixed with an CpG oligodeoxynucleotide (ODN) to form a stabilized immunostimulatory complex.

17. An isolated antibody or epitope-binding fragment thereof that specifically binds to the B cell epitope of the DPR peptide immunogen construct of claim 1.

18. The isolated antibody or epitope-binding fragment thereof according to claim 17 bound to the DPR peptide immunogen construct.

19. An isolated antibody or epitope-biding fragment thereof that specifically binds to the B cell epitope of the DPR peptide immunogen construct of claim 9.

20. A composition comprising the isolated antibody or epitope-binding fragment thereof according to claim 17.

21. A method for producing antibodies that recognize DPR proteins in a host comprising administering to the host a composition comprising the DPR peptide immunogen construct of claim 1 and a delivery vehicle and/or adjuvant.

22. A method for reducing the amount of DPR proteins in an animal comprising administering a pharmacologically effective amount of the DPR peptide immunogen of claim 1 to the animal.

23. The method of claim 22, wherein the animal is a human.

24. A method for identifying DPR proteins in a biological sample comprising:
a. exposing the biological sample to the antibody or epitope-binding fragment thereof according to claim 17 under conditions that allow the antibody or epitope-binding fragment thereof to bind to a DPR protein; and
b. detecting the amount of the antibody or epitope-binding fragment thereof bound to the DPR protein in the biological sample.


SEQUENCE LISTING (232 sequences)

SEQ ID NO: 1 - protein, 20 aa, Homo sapiens
  PEPTIDE (1)..(20): (GA)10
GAGAGAGAGAGAGAGAGAGA

SEQ ID NO: 2 - protein, 30 aa, Rattus norvegicus
  PEPTIDE (1)..(30): (GA)15
GAGAGAGAGAGAGAGAGAGAGAGAGAGAGA

SEQ ID NO: 3 - protein, 70 aa, Homo sapiens
  PEPTIDE (1)..(50): (GA)25
GAGAGAGAGAGAGAGAGAGAGAGAGAGAGAGAGAGAGAGAGAGAGAGAGAGPGPGPGPGPGPGPGPGPGP

SEQ ID NO: 4 - protein, 20 aa, Homo sapiens
  PEPTIDE (1)..(20): (GP)10
GPGPGPGPGPGPGPGPGPGP

SEQ ID NO: 5 - protein, 30 aa, Homo sapiens
  PEPTIDE (1)..(30): (GP)15
GPGPGPGPGPGPGPGPGPGPGPGPGPGPGP

SEQ ID NO: 6 - protein, 50 aa, Homo sapiens
  PEPTIDE (1)..(50): (GP)25
GPGPGPGPGPGPGPGPGPGPGPGPGPGPGPGPGPGPGPGPGPGPGPGPGP

SEQ ID NO: 7 - protein, 20 aa, Homo sapiens
  PEPTIDE (1)..(20): (GR)10
GRGRGRGRGRGRGRGRGRGR

SEQ ID NO: 8 - protein, 30 aa, Homo sapiens
  PEPTIDE (1)..(30): (GR)15
GRGRGRGRGRGRGRGRGRGRGRGRGRGRGR

SEQ ID NO: 9 - protein, 50 aa, Homo sapiens
  PEPTIDE (1)..(50): (GR)25
GRGRGRGRGRGRGRGRGRGRGRGRGRGRGRGRGRGRGRGRGRGRGRGRGR

SEQ ID NO: 10 - protein, 20 aa, Homo sapiens
  PEPTIDE (1)..(20): (PR)10
PRPRPRPRPRPRPRPRPRPR

SEQ ID NO: 11 - protein, 30 aa, Homo sapiens
  PEPTIDE (1)..(30): (PR)15
PRPRPRPRPRPRPRPRPRPRPRPRPRPRPR

SEQ ID NO: 12 - protein, 50 aa, Homo sapiens
  PEPTIDE (1)..(50): (PR)25
PRPRPRPRPRPRPRPRPRPRPRPRPRPRPRPRPRPRPRPRPRPRPRPRPR

SEQ ID NO: 13 - protein, 20 aa, Homo sapiens
  PEPTIDE (1)..(20): (PA)10
PAPAPAPAPAPAPAPAPAPA

SEQ ID NO: 14 - protein, 30 aa, Homo sapiens
  PEPTIDE (1)..(30): (PA)15
PAPAPAPAPAPAPAPAPAPAPAPAPAPAPA

SEQ ID NO: 15 - protein, 50 aa, Homo sapiens
  PEPTIDE (1)..(50): (PA)25
PAPAPAPAPAPAPAPAPAPAPAPAPAPAPAPAPAPAPAPAPAPAPAPAPA

SEQ ID NO: 16 - protein, 16 aa, Measles virus
  PEPTIDE (1)..(16): MvF Th (SSAL1 Th1)
DLSDLKGLLLHKLDGL

SEQ ID NO: 17 - protein, 16 aa, Measles virus
  PEPTIDE (1)..(16): MvF Th (SSAL1 Th1)
EISEIRGIIIHRIEGI

SEQ ID NO: 18 - protein, 16 aa, Measles virus
  PEPTIDE (1)..(16): MvF Th (SSAL1 Th1)
DVSDVKGVVVHKVDGV

SEQ ID NO: 19 - protein, 16 aa, Measles virus
  PEPTIDE (1)..(16): MvF Th (SSAL1 Th1)
DFSDFKGFFFHKFDGF

SEQ ID NO: 20 - protein, 16 aa, Measles virus
  PEPTIDE (1)..(16): MvF Th (SSAL1 Th1)
  SITE (1)..(1): D or E
  SITE (2)..(2): L or I or V or F
  SITE (4)..(4): D or E
  SITE (5)..(5): L or I or V or F
  SITE (6)..(6): K or R
  SITE (8)..(8): L or I or V or F
  SITE (9)..(9): L or I or V or F
  SITE (10)..(10): L or I or V or F
  SITE (12)..(12): K or R
  SITE (13)..(13): L or I or V or F
  SITE (14)..(14): D or E
  SITE (16)..(16): L or I or V or F
XXSXXXGXXXHXXXGX

SEQ ID NO: 21 - protein, 15 aa, Measles virus
  PEPTIDE (1)..(15): MvF1 Th (UBITh5)
LSEIKGVIVHRLEGV

SEQ ID NO: 22 - protein, 15 aa, Measles virus
  PEPTIDE (1)..(15): MvF 2 Th
ISEIKGVIVHKIEGI

SEQ ID NO: 23 - protein, 19 aa, Measles virus
  PEPTIDE (1)..(19): MvF3 Th
ISISEIKGVIVHKIEGILF

SEQ ID NO: 24 - protein, 19 aa, Measles virus
  PEPTIDE (1)..(19): MvF3 Th
ISITEIRTVIVTRIETILF

SEQ ID NO: 25 - protein, 19 aa, Measles virus
  PEPTIDE (1)..(19): MvF 3 Th
  SITE (4)..(4): S or T
  SITE (7)..(7): K or R
  SITE (8)..(8): G or T
  SITE (12)..(12): H or T
  SITE (13)..(13): K or R
  SITE (16)..(16): G or T
ISIXEIXXVIVXXIEXILF

SEQ ID NO: 26 - protein, 22 aa, Measles virus
  PEPTIDE (1)..(22): KKKMvF3 Th
KKKISISEIKGVIVHKIEGILF

SEQ ID NO: 27 - protein, 22 aa, Measles virus
  PEPTIDE (1)..(22): KKKMvF3 Th
KKKISITEIRTVIVTRIETILF

SEQ ID NO: 28 - protein, 22 aa, Measles virus
  PEPTIDE (1)..(22): KKKMvF 3 Th
  SITE (7)..(7): S or T
  SITE (10)..(10): K or R
  SITE (11)..(11): G or T
  SITE (15)..(15): H or T
  SITE (16)..(16): K or R
  SITE (19)..(19): G or T
KKKISIXEIXXVIVXXIEXILF

SEQ ID NO: 29 - protein, 19 aa, Measles virus
  PEPTIDE (1)..(19): MvF4 Th  (UBITh3)
ISISEIKGVIVHKIETILF

SEQ ID NO: 30 - protein, 19 aa, Measles virus
  PEPTIDE (1)..(19): MvF4 Th  (UBITh3)
ISITEIRTVIVTRIETILF

SEQ ID NO: 31 - protein, 19 aa, Measles virus
  PEPTIDE (1)..(19): MvF4 Th  (UBITh3)
  SITE (4)..(4): S or T
  SITE (7)..(7): K or R
  SITE (8)..(8): G or T
  SITE (12)..(12): H or T
  SITE (13)..(13): K or R
ISIXEIXXVIVXXIETILF

SEQ ID NO: 32 - protein, 19 aa, Measles virus
  PEPTIDE (1)..(19): MvF5 Th (UBITh1)
ISITEIKGVIVHRIETILF

SEQ ID NO: 33 - protein, 22 aa, Measles virus
  PEPTIDE (1)..(22): KKKMvF5 Th (UBITh1a)
KKKISITEIKGVIVHRIETILF

SEQ ID NO: 34 - protein, 18 aa, Hepatitis B virus
  PEPTIDE (1)..(18): HBsAg1 Th (SSAL2 Th2)
KKKLFLLTKLLTLPQSLD

SEQ ID NO: 35 - protein, 18 aa, Hepatitis B virus
  PEPTIDE (1)..(18): HBsAg1 Th (SSAL2 Th2)
RRRIKIITRIITIPLSIR

SEQ ID NO: 36 - protein, 18 aa, Hepatitis B virus
  PEPTIDE (1)..(18): HBsAg1 Th (SSAL2 Th2)
KKKVRVVTKVVTVPISVD

SEQ ID NO: 37 - protein, 18 aa, Hepatitis B virus
  PEPTIDE (1)..(18): HBsAg1 Th (SSAL2 Th2)
KKKFFFFTKFFTFPVSFD

SEQ ID NO: 38 - protein, 18 aa, Hepatitis B virus
  PEPTIDE (1)..(18): HBsAg1 Th (SSAL2 Th2)
KKKLFLLTKLLTLPFSLD

SEQ ID NO: 39 - protein, 18 aa, Hepatitis B virus
  PEPTIDE (1)..(18): HBsAg1 Th (SSAL2 Th2)
  SITE (1)..(1): K or R
  SITE (2)..(2): K or R
  SITE (3)..(3): K or R
  SITE (4)..(4): L or I or V or F
  SITE (5)..(5): F or K or R
  SITE (6)..(6): L or I or V or F
  SITE (7)..(7): L or I or V or F
  SITE (9)..(9): K or R
  SITE (10)..(10): L or I or V or F
  SITE (11)..(11): L or I or V or F
  SITE (13)..(13): L or I or V or F
  SITE (15)..(15): Q or L or I or V or F
  SITE (17)..(17): L or I or V or F
  SITE (18)..(18): D or R
XXXXXXXTXXXTXPXSXX

SEQ ID NO: 40 - protein, 18 aa, Hepatitis B virus
  PEPTIDE (1)..(18): HBsAg2 Th
KKKIITITRIITIPQSLD

SEQ ID NO: 41 - protein, 18 aa, Hepatitis B virus
  PEPTIDE (1)..(18): HBsAg2 Th
KKKFFLLTRILTIITTID

SEQ ID NO: 42 - protein, 18 aa, Hepatitis B virus
  PEPTIDE (1)..(18): HBsAg 2 Th
  SITE (4)..(4): I or F
  SITE (5)..(5): I or F
  SITE (6)..(6): T or L
  SITE (7)..(7): I or L
  SITE (11)..(11): I or L
  SITE (14)..(14): P or I
  SITE (15)..(15): Q or T
  SITE (16)..(16): S or T
  SITE (17)..(17): L or I
KKKXXXXTRIXTIXXXXD

SEQ ID NO: 43 - protein, 18 aa, Hepatitis B virus
  PEPTIDE (1)..(18): HBsAg 3 Th
KKKIITITRIITIITTID

SEQ ID NO: 44 - protein, 15 aa, Hepatitis B virus
  PEPTIDE (1)..(15): HBsAg4 Th (UBITh4)
FFLLTRILTIPQSLD

SEQ ID NO: 45 - protein, 18 aa, Hepatitis B virus
  PEPTIDE (1)..(18): KKK-HBsAg4 Th
KKKFFLLTRILTIPQSLD

SEQ ID NO: 46 - protein, 14 aa, Hepatitis B virus
  PEPTIDE (1)..(14): HBsAg Th
FFLLTRILTIPQSL

SEQ ID NO: 47 - protein, 24 aa, Bordetella pertussis
  PEPTIDE (1)..(24): Bordetella pertussis Th (UBITh7)
GAYARCPNGTRALTVAELRGNAEL

SEQ ID NO: 48 - protein, 25 aa, Cholera Toxin
  PEPTIDE (1)..(25): Cholera Toxin Th
ALNIWDRFDVFCTLGATTGYLKGNS

SEQ ID NO: 49 - protein, 15 aa, Clostridium tetani
  PEPTIDE (1)..(15): Clostridium tetani TT1 Th
QYIKANSKFIGITEL

SEQ ID NO: 50 - protein, 17 aa, Clostridium tetani
  PEPTIDE (1)..(17): Clostridium tetani1 Th (UBITh6)
KKQYIKANSKFIGITEL

SEQ ID NO: 51 - protein, 21 aa, Clostridium tetani
  PEPTIDE (1)..(21): Clostridium tetani  TT2 Th
FNNFTVSFWLRVPKVSASHLE

SEQ ID NO: 52 - protein, 16 aa, Clostridium tetani
  PEPTIDE (1)..(16): Clostridium tetani TT3 Th
KFIIKRYTPNNEIDSF

SEQ ID NO: 53 - protein, 16 aa, Clostridium tetani
  PEPTIDE (1)..(16): Clostridium tetani  TT4 Th
VSIDKFRIFCKALNPK

SEQ ID NO: 54 - protein, 17 aa, Clostridium tetani
  PEPTIDE (1)..(17): Clostridium tetani 2 Th
WVRDIIDDFTNESSQKT

SEQ ID NO: 55 - protein, 23 aa, diphtheria bacilli
  PEPTIDE (1)..(23): Diphtheria Th
DSETADNLEKTVAALSILPGHGC

SEQ ID NO: 56 - protein, 19 aa, Epstein-Barr virus
  PEPTIDE (1)..(19): EBV BHRF1 Th
AGLTLSLLVICSYLFISRG

SEQ ID NO: 57 - protein, 20 aa, Epstein-Barr virus
  PEPTIDE (1)..(20): EBV EBNA-1 Th
PGPLRESIVCYFMVFLQTHI

SEQ ID NO: 58 - protein, 18 aa, Epstein-Barr virus
  PEPTIDE (1)..(18): EBV CP Th
VPGLYSPCRAFFNKEELL

SEQ ID NO: 59 - protein, 15 aa, Epstein-Barr virus
  PEPTIDE (1)..(15): EBV GP340 Th
TGHGARTSTEPTTDY

SEQ ID NO: 60 - protein, 13 aa, Epstein-Barr virus
  PEPTIDE (1)..(13): EBV BPLF1 Th
KELKRQYEKKLRQ

SEQ ID NO: 61 - protein, 11 aa, Epstein-Barr virus
  PEPTIDE (1)..(11): EBV EBNA-2 Th
TVFYNIPPMPL

SEQ ID NO: 62 - protein, 14 aa, Human cytomegalovirus
  PEPTIDE (1)..(14): HCMV IE1 Th
DKREMWMACIKELH

SEQ ID NO: 63 - protein, 11 aa, Influenza virus
  PEPTIDE (1)..(11): Influenza Matrix protein 1_1 Th
FVFTLTVPSER

SEQ ID NO: 64 - protein, 15 aa, Influenza virus
  PEPTIDE (1)..(15): Influenza Matrix protein 1_2 Th
SGPLKAEIAQRLEDV

SEQ ID NO: 65 - protein, 9 aa, Influenza virus
  PEPTIDE (1)..(9): Influenza Non-structural protein 1 Th
DRLRRDQKS

SEQ ID NO: 66 - protein, 21 aa, Plasmodium falciparum
  PEPTIDE (1)..(21): Plasmodium falciparum Th
DHEKKHAKMEKASSVFNVVNS

SEQ ID NO: 67 - protein, 17 aa, Schistosoma mansoni
  PEPTIDE (1)..(17): Schistosoma mansoni Th
KWFKTNAPNGVDEKHRH

SEQ ID NO: 68 - protein, 43 aa, Artificial Sequence
  Synthetic Peptide
  PEPTIDE (1)..(20): (GA)10
  PEPTIDE (21)..(24): KKK-epsilon K as a spacer
  SITE (24)..(24): epsilon K
  PEPTIDE (25)..(43): MvF5 Th (UBITh1)
GAGAGAGAGAGAGAGAGAGAKKKKISITEIKGVIVHRIETILF

SEQ ID NO: 69 - protein, 53 aa, Artificial Sequence
  Synthetic Peptide
  PEPTIDE (1)..(30): (GA)15
  PEPTIDE (31)..(34): KKK-epsilon K as a spacer
  SITE (34)..(34): epsilon K
  PEPTIDE (35)..(53): MvF5 Th (UBITh1)
GAGAGAGAGAGAGAGAGAGAGAGAGAGAGAKKKKISITEIKGVIVHRIETILF

SEQ ID NO: 70 - protein, 73 aa, Artificial Sequence
  Synthetic Peptide
  PEPTIDE (1)..(50): (GA)25
  PEPTIDE (51)..(54): KKK-epsilon K as a spacer
  SITE (54)..(54): epsilon K
  PEPTIDE (55)..(73): MvF5 Th (UBITh1)
GAGAGAGAGAGAGAGAGAGAGAGAGAGAGAGAGAGAGAGAGAGAGAGAGAKKKKISITEIKGVIVHRIETILF

SEQ ID NO: 71 - protein, 40 aa, Artificial Sequence
  Synthetic Peptide
  PEPTIDE (1)..(20): (GA)10
  SITE (21)..(21): epsilon K
  PEPTIDE (22)..(40): MvF5 Th (UBITh1)
GAGAGAGAGAGAGAGAGAGAKISITEIKGVIVHRIETILF

SEQ ID NO: 72 - protein, 50 aa, Artificial Sequence
  Synthetic Peptide
  PEPTIDE (1)..(30): (GA)15
  SITE (31)..(31): epsilon K as a spacer
  PEPTIDE (32)..(50): MvF5 Th (UBITh1)
GAGAGAGAGAGAGAGAGAGAGAGAGAGAGAKISITEIKGVIVHRIETILF

SEQ ID NO: 73 - protein, 70 aa, Artificial Sequence
  Synthetic Peptide
  PEPTIDE (1)..(50): (GA)25
  SITE (51)..(51): epsilon K as a spacer
  PEPTIDE (52)..(70): MvF5 Th (UBITh1)
GAGAGAGAGAGAGAGAGAGAGAGAGAGAGAGAGAGAGAGAGAGAGAGAGAKISITEIKGVIVHRIETILF

SEQ ID NO: 74 - protein, 43 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): (GA)10
ISITEIKGVIVHRIETILFKKKKGAGAGAGAGAGAGAGAGAGA

SEQ ID NO: 75 - protein, 53 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(53): (GA)15
ISITEIKGVIVHRIETILFKKKKGAGAGAGAGAGAGAGAGAGAGAGAGAGAGA

SEQ ID NO: 76 - protein, 73 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(73): (GA)25
ISITEIKGVIVHRIETILFKKKKGAGAGAGAGAGAGAGAGAGAGAGAGAGAGAGAGAGAGAGAGAGAGAGAGA

SEQ ID NO: 77 - protein, 40 aa, Artificial Sequence
  Synthetic Peptide
  PEPTIDE (1)..(19): MvF5 Th (UBITh1)
  SITE (20)..(20): epsilon-K
  PEPTIDE (21)..(40): (GA)10
ISITEIKGVIVHRIETILFKGAGAGAGAGAGAGAGAGAGA

SEQ ID NO: 78 - protein, 50 aa, Artificial Sequence
  Synthetic Peptide
  PEPTIDE (1)..(19): MvF5 Th (UBITh1)
  SITE (20)..(20): epsilon-K
  PEPTIDE (21)..(50): (GA)15
ISITEIKGVIVHRIETILFKGAGAGAGAGAGAGAGAGAGAGAGAGAGAGA

SEQ ID NO: 79 - protein, 70 aa, Artificial Sequence
  Synthetic Peptide
  PEPTIDE (1)..(19): MvF5 Th (UBITh1)
  SITE (20)..(20): epsilon-K
  PEPTIDE (21)..(70): (GA)25
ISITEIKGVIVHRIETILFKGAGAGAGAGAGAGAGAGAGAGAGAGAGAGAGAGAGAGAGAGAGAGAGAGA

SEQ ID NO: 80 - protein, 66 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): (GA)10
  PEPTIDE (44)..(47): epsilon K-KKK as a spacer
  SITE (47)..(47): epsilon-K
  PEPTIDE (48)..(66): MvF5 Th (UBITh1)
ISITEIKGVIVHRIETILFKKKKGAGAGAGAGAGAGAGAGAGAKKKKISITEIKGVIVHRIETILF

SEQ ID NO: 81 - protein, 76 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(53): (GA)15
  PEPTIDE (54)..(57): epsilon K-KKK as a spacer
  SITE (57)..(57): epsilon-K
  PEPTIDE (58)..(76): MvF5 Th (UBITh1)
ISITEIKGVIVHRIETILFKKKKGAGAGAGAGAGAGAGAGAGAGAGAGAGAGAKKKKISITEIKGVIVHRIETILF

SEQ ID NO: 82 - protein, 96 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(73): (GA)25
  PEPTIDE (74)..(77): epsilon K-KKK as a spacer
  SITE (77)..(77): epsilon-K
  PEPTIDE (78)..(96): MvF5 Th (UBITh1)
ISITEIKGVIVHRIETILFKKKKGAGAGAGAGAGAGAGAGAGAGAGAGAGAGAGAGAGAGAGAGAGAGAGAGAKKKKISITEIKGVIVHRIETILF

SEQ ID NO: 83 - protein, 60 aa, Artificial Sequence
  Synthetic Peptide
  PEPTIDE (1)..(19): MvF5 Th (UBITh1)
  SITE (20)..(20): epsilon-K as a spacer
  PEPTIDE (21)..(40): (GA)10
  SITE (41)..(41): epsilon-K
  PEPTIDE (42)..(60): MvF5 Th (UBITh1)
ISITEIKGVIVHRIETILFKGAGAGAGAGAGAGAGAGAGAKISITEIKGVIVHRIETILF

SEQ ID NO: 84 - protein, 70 aa, Artificial Sequence
  Synthetic Peptide
  PEPTIDE (1)..(19): MvF5 Th (UBITh1)
  SITE (20)..(20): epsilon K as a spacer
  PEPTIDE (21)..(50): (GA)15
  SITE (51)..(51): epsilon K as a spacer
  PEPTIDE (52)..(70): MvF5 Th (UBITh1)
ISITEIKGVIVHRIETILFKGAGAGAGAGAGAGAGAGAGAGAGAGAGAGAKISITEIKGVIVHRIETILF

SEQ ID NO: 85 - protein, 90 aa, Artificial Sequence
  Synthetic Peptide
  PEPTIDE (1)..(19): MvF5 Th (UBITh1)
  SITE (20)..(20): epsilon K as a spacer
  PEPTIDE (21)..(70): (GA)25
  SITE (71)..(71): epsilon K as a spacer
  PEPTIDE (72)..(90): MvF5 Th (UBITh1)
ISITEIKGVIVHRIETILFKGAGAGAGAGAGAGAGAGAGAGAGAGAGAGAGAGAGAGAGAGAGAGAGAGAKISITEIKGVIVHRIETILF

SEQ ID NO: 86 - protein, 66 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(43): (GA)10
  PEPTIDE (44)..(47): epsilon K-KKK as a spacer
  SITE (47)..(47): epsilon-K
  PEPTIDE (48)..(66): MvF5 Th (UBITh1)
ISIXEIXXVIVXXIETILFKKKKGAGAGAGAGAGAGAGAGAGAKKKKISITEIKGVIVHRIETILF

SEQ ID NO: 87 - protein, 76 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(53): (GA)15
  PEPTIDE (54)..(57): epsilon K-KKK as a spacer
  SITE (57)..(57): epsilon-K
  PEPTIDE (58)..(76): MvF5 Th (UBITh1)
ISIXEIXXVIVXXIETILFKKKKGAGAGAGAGAGAGAGAGAGAGAGAGAGAGAKKKKISITEIKGVIVHRIETILF

SEQ ID NO: 88 - protein, 96 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(73): (GA)25
  PEPTIDE (74)..(77): epsilon K-KKK as a spacer
  SITE (77)..(77): epsilon-K
  PEPTIDE (78)..(96): MvF5 Th (UBITh1)
ISIXEIXXVIVXXIETILFKKKKGAGAGAGAGAGAGAGAGAGAGAGAGAGAGAGAGAGAGAGAGAGAGAGAGAKKKKISITEIKGVIVHRIETILF

SEQ ID NO: 89 - protein, 60 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(40): (GA)10
  SITE (41)..(41): epsilon-K as a spacer
  PEPTIDE (42)..(60): MvF5 Th (UBITh1)
ISIXEIXXVIVXXIETILFKGAGAGAGAGAGAGAGAGAGAKISITEIKGVIVHRIETILF

SEQ ID NO: 90 - protein, 70 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(50): (GA)15
  SITE (51)..(51): epsilon-K as a spacer
  PEPTIDE (52)..(70): MvF5 Th (UBITh1)
ISIXEIXXVIVXXIETILFKGAGAGAGAGAGAGAGAGAGAGAGAGAGAGAKISITEIKGVIVHRIETILF

SEQ ID NO: 91 - protein, 90 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(70): (GA)25
  SITE (71)..(71): epsilon-K as a spacer
  PEPTIDE (72)..(90): MvF5 Th (UBITh1)
ISIXEIXXVIVXXIETILFKGAGAGAGAGAGAGAGAGAGAGAGAGAGAGAGAGAGAGAGAGAGAGAGAGAKISITEIKGVIVHRIETILF

SEQ ID NO: 92 - protein, 66 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): (GA)10
  PEPTIDE (44)..(47): epsilon K-KKK as a spacer
  SITE (47)..(47): epsilon-K
  PEPTIDE (48)..(66): UBITh3
  SITE (51)..(51): S or T
  SITE (54)..(54): K or R
  SITE (55)..(55): G or T
  SITE (59)..(59): H or T
  SITE (60)..(60): K or R
ISITEIKGVIVHRIETILFKKKKGAGAGAGAGAGAGAGAGAGAKKKKISIXEIXXVIVXXIETILF

SEQ ID NO: 93 - protein, 76 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(53): (GA)15
  PEPTIDE (54)..(57): epsilon K-KKK as a spacer
  SITE (57)..(57): epsilon-K
  PEPTIDE (58)..(76): UBITh3
  SITE (61)..(61): S or T
  SITE (64)..(64): K or R
  SITE (65)..(65): G or T
  SITE (69)..(69): H or T
  SITE (70)..(70): K or R
ISITEIKGVIVHRIETILFKKKKGAGAGAGAGAGAGAGAGAGAGAGAGAGAGAKKKKISIXEIXXVIVXXIETILF

SEQ ID NO: 94 - protein, 96 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(73): (GA)25
  PEPTIDE (74)..(77): epsilon K-KKK as a spacer
  SITE (77)..(77): epsilon-K
  PEPTIDE (78)..(96): UBITh3
  SITE (81)..(81): S or T
  SITE (84)..(84): K or R
  SITE (85)..(85): G or T
  SITE (89)..(89): H or T
  SITE (90)..(90): K or R
ISITEIKGVIVHRIETILFKKKKGAGAGAGAGAGAGAGAGAGAGAGAGAGAGAGAGAGAGAGAGAGAGAGAGAKKKKISIXEIXXVIVXXIETILF

SEQ ID NO: 95 - protein, 60 aa, Artificial Sequence
  Synthetic Peptide
  PEPTIDE (1)..(19): MvF5 Th (UBITh1)
  SITE (20)..(20): epsilon K as a spacer
  PEPTIDE (21)..(40): (GA)10
  SITE (41)..(41): epsilon-K as a spacer
  PEPTIDE (42)..(60): UBITh3
  SITE (45)..(45): S or T
  SITE (48)..(48): K or R
  SITE (49)..(49): G or T
  SITE (53)..(53): H or T
  SITE (54)..(54): K or R
ISITEIKGVIVHRIETILFKGAGAGAGAGAGAGAGAGAGAKISIXEIXXVIVXXIETILF

SEQ ID NO: 96 - protein, 70 aa, Artificial Sequence
  Synthetic Peptide
  PEPTIDE (1)..(19): MvF5 Th (UBITh1)
  SITE (20)..(20): epsilon-K as a spacer
  PEPTIDE (21)..(50): (GA)15
  SITE (51)..(51): epsilon-K as a spacer
  PEPTIDE (52)..(70): UBITh3
  SITE (55)..(55): S or T
  SITE (58)..(58): K or R
  SITE (59)..(59): G or T
  SITE (63)..(63): H or T
  SITE (64)..(64): K or R
ISITEIKGVIVHRIETILFKGAGAGAGAGAGAGAGAGAGAGAGAGAGAGAKISIXEIXXVIVXXIETILF

SEQ ID NO: 97 - protein, 90 aa, Artificial Sequence
  Synthetic Peptide
  PEPTIDE (1)..(19): MvF5 Th (UBITh1)
  SITE (20)..(20): epsilon-K as a spacer
  PEPTIDE (21)..(70): (GA)25
  SITE (71)..(71): epsilon-K as a spacer
  PEPTIDE (72)..(90): UBITh3
  SITE (75)..(75): S or T
  SITE (78)..(78): K or R
  SITE (79)..(79): G or T
  SITE (83)..(83): H or T
  SITE (84)..(84): K or R
ISITEIKGVIVHRIETILFKGAGAGAGAGAGAGAGAGAGAGAGAGAGAGAGAGAGAGAGAGAGAGAGAGAKISIXEIXXVIVXXIETILF

SEQ ID NO: 98 - protein, 43 aa, Artificial Sequence
  Synthetic Peptide
  PEPTIDE (1)..(20): (GP)10
  PEPTIDE (21)..(24): KKK-epsilon K as a spacer
  SITE (24)..(24): epsilon K
  PEPTIDE (25)..(43): MvF5 Th (UBITh1)
GPGPGPGPGPGPGPGPGPGPKKKKISITEIKGVIVHRIETILF

SEQ ID NO: 99 - protein, 53 aa, Artificial Sequence
  Synthetic Peptide
  PEPTIDE (1)..(30): (GP)15
  PEPTIDE (31)..(34): KKK-epsilon K as a spacer
  SITE (34)..(34): epsilon K
  PEPTIDE (35)..(53): MvF5 Th (UBITh1)
GPGPGPGPGPGPGPGPGPGPGPGPGPGPGPKKKKISITEIKGVIVHRIETILF

SEQ ID NO: 100 - protein, 73 aa, Artificial Sequence
  Synthetic Peptide
  PEPTIDE (1)..(50): (GP)25
  PEPTIDE (51)..(54): KKK-epsilon K as a spacer
  SITE (54)..(54): epsilon K
  PEPTIDE (55)..(73): MvF5 Th (UBITh1)
GPGPGPGPGPGPGPGPGPGPGPGPGPGPGPGPGPGPGPGPGPGPGPGPGPKKKKISITEIKGVIVHRIETILF

SEQ ID NO: 101 - protein, 40 aa, Artificial Sequence
  Synthetic Peptide
  PEPTIDE (1)..(20): (GP)10
  SITE (21)..(21): epsilon K
  PEPTIDE (22)..(40): MvF5 Th (UBITh1)
GPGPGPGPGPGPGPGPGPGPKISITEIKGVIVHRIETILF

SEQ ID NO: 102 - protein, 50 aa, Artificial Sequence
  Synthetic Peptide
  PEPTIDE (1)..(30): (GP)15
  SITE (31)..(31): epsilon K as a spacer
  PEPTIDE (32)..(50): MvF5 Th (UBITh1)
GPGPGPGPGPGPGPGPGPGPGPGPGPGPGPKISITEIKGVIVHRIETILF

SEQ ID NO: 103 - protein, 70 aa, Artificial Sequence
  Synthetic Peptide
  PEPTIDE (1)..(50): (GP)25
  SITE (51)..(51): epsilon K as a spacer
  PEPTIDE (52)..(70): MvF5 Th (UBITh1)
GPGPGPGPGPGPGPGPGPGPGPGPGPGPGPGPGPGPGPGPGPGPGPGPGPKISITEIKGVIVHRIETILF

SEQ ID NO: 104 - protein, 43 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): (GP)10
ISITEIKGVIVHRIETILFKKKKGPGPGPGPGPGPGPGPGPGP

SEQ ID NO: 105 - protein, 53 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(53): (GP)15
ISITEIKGVIVHRIETILFKKKKGPGPGPGPGPGPGPGPGPGPGPGPGPGPGP

SEQ ID NO: 106 - protein, 73 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(73): (GP)25
ISITEIKGVIVHRIETILFKKKKGPGPGPGPGPGPGPGPGPGPGPGPGPGPGPGPGPGPGPGPGPGPGPGPGP

SEQ ID NO: 107 - protein, 40 aa, Artificial Sequence
  Synthetic Peptide
  PEPTIDE (1)..(19): MvF5 Th (UBITh1)
  SITE (20)..(20): epsilon-K
  PEPTIDE (21)..(40): (GP)10
ISITEIKGVIVHRIETILFKGPGPGPGPGPGPGPGPGPGP

SEQ ID NO: 108 - protein, 50 aa, Artificial Sequence
  Synthetic Peptide
  PEPTIDE (1)..(19): MvF5 Th (UBITh1)
  SITE (20)..(20): epsilon-K
  PEPTIDE (21)..(50): (GP)15
ISITEIKGVIVHRIETILFKGPGPGPGPGPGPGPGPGPGPGPGPGPGPGP

SEQ ID NO: 109 - protein, 70 aa, Artificial Sequence
  Synthetic Peptide
  PEPTIDE (1)..(19): MvF5 Th (UBITh1)
  SITE (20)..(20): epsilon-K
  PEPTIDE (21)..(70): (GP)25
ISITEIKGVIVHRIETILFKGPGPGPGPGPGPGPGPGPGPGPGPGPGPGPGPGPGPGPGPGPGPGPGPGP

SEQ ID NO: 110 - protein, 66 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): (GP)10
  PEPTIDE (44)..(47): epsilon K-KKK as a spacer
  SITE (47)..(47): epsilon-K
  PEPTIDE (48)..(66): MvF5 Th (UBITh1)
ISITEIKGVIVHRIETILFKKKKGPGPGPGPGPGPGPGPGPGPKKKKISITEIKGVIVHRIETILF

SEQ ID NO: 111 - protein, 76 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(53): (GP)15
  PEPTIDE (54)..(57): epsilon K-KKK as a spacer
  SITE (57)..(57): epsilon-K
  PEPTIDE (58)..(76): MvF5 Th (UBITh1)
ISITEIKGVIVHRIETILFKKKKGPGPGPGPGPGPGPGPGPGPGPGPGPGPGPKKKKISITEIKGVIVHRIETILF

SEQ ID NO: 112 - protein, 96 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(73): (GP)25
  PEPTIDE (74)..(77): epsilon K-KKK as a spacer
  SITE (77)..(77): epsilon-K
  PEPTIDE (78)..(96): MvF5 Th (UBITh1)
ISITEIKGVIVHRIETILFKKKKGPGPGPGPGPGPGPGPGPGPGPGPGPGPGPGPGPGPGPGPGPGPGPGPGPKKKKISITEIKGVIVHRIETILF

SEQ ID NO: 113 - protein, 60 aa, Artificial Sequence
  Synthetic Peptide
  PEPTIDE (1)..(19): MvF5 Th (UBITh1)
  SITE (20)..(20): epsilon-K as a spacer
  PEPTIDE (21)..(40): (GP)10
  SITE (41)..(41): epsilon-K
  PEPTIDE (42)..(60): MvF5 Th (UBITh1)
ISITEIKGVIVHRIETILFKGPGPGPGPGPGPGPGPGPGPKISITEIKGVIVHRIETILF

SEQ ID NO: 114 - protein, 70 aa, Artificial Sequence
  Synthetic Peptide
  PEPTIDE (1)..(19): MvF5 Th (UBITh1)
  SITE (20)..(20): epsilon K as a spacer
  PEPTIDE (21)..(50): GP)15
  SITE (51)..(51): epsilon K as a spacer
  PEPTIDE (52)..(70): MvF5 Th (UBITh1)
ISITEIKGVIVHRIETILFKGPGPGPGPGPGPGPGPGPGPGPGPGPGPGPKISITEIKGVIVHRIETILF

SEQ ID NO: 115 - protein, 90 aa, Artificial Sequence
  Synthetic Peptide
  PEPTIDE (1)..(19): MvF5 Th (UBITh1)
  SITE (20)..(20): epsilon K as a spacer
  PEPTIDE (21)..(70): (GP)25
  SITE (71)..(71): epsilon K as a spacer
  PEPTIDE (72)..(90): MvF5 Th (UBITh1)
ISITEIKGVIVHRIETILFKGPGPGPGPGPGPGPGPGPGPGPGPGPGPGPGPGPGPGPGPGPGPGPGPGPKISITEIKGVIVHRIETILF

SEQ ID NO: 116 - protein, 66 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(43): (GP)10
  PEPTIDE (44)..(47): epsilon K-KKK as a spacer
  SITE (47)..(47): epsilon-K
  PEPTIDE (48)..(66): MvF5 Th (UBITh1)
ISIXEIXXVIVXXIETILFKKKKGPGPGPGPGPGPGPGPGPGPKKKKISITEIKGVIVHRIETILF

SEQ ID NO: 117 - protein, 76 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(53): (GP)15
  PEPTIDE (54)..(57): epsilon K-KKK as a spacer
  SITE (57)..(57): epsilon-K
  PEPTIDE (58)..(76): MvF5 Th (UBITh1)
ISIXEIXXVIVXXIETILFKKKKGPGPGPGPGPGPGPGPGPGPGPGPGPGPGPKKKKISITEIKGVIVHRIETILF

SEQ ID NO: 118 - protein, 96 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(73): (GP)25
  PEPTIDE (74)..(77): epsilon K-KKK as a spacer
  SITE (77)..(77): epsilon-K
  PEPTIDE (78)..(96): MvF5 Th (UBITh1)
ISIXEIXXVIVXXIETILFKKKKGPGPGPGPGPGPGPGPGPGPGPGPGPGPGPGPGPGPGPGPGPGPGPGPGPKKKKISITEIKGVIVHRIETILF

SEQ ID NO: 119 - protein, 60 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(40): (GP)10
  SITE (41)..(41): epsilon-K as a spacer
  PEPTIDE (42)..(60): MvF5 Th (UBITh1)
ISIXEIXXVIVXXIETILFKGPGPGPGPGPGPGPGPGPGPKISITEIKGVIVHRIETILF

SEQ ID NO: 120 - protein, 70 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(50): (GP)15
  SITE (51)..(51): epsilon-K as a spacer
  PEPTIDE (52)..(70): MvF5 Th (UBITh1)
ISIXEIXXVIVXXIETILFKGPGPGPGPGPGPGPGPGPGPGPGPGPGPGPKISITEIKGVIVHRIETILF

SEQ ID NO: 121 - protein, 90 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(70): (GP)25
  SITE (71)..(71): epsilon-K as a spacer
  PEPTIDE (72)..(90): MvF5 Th (UBITh1)
ISIXEIXXVIVXXIETILFKGPGPGPGPGPGPGPGPGPGPGPGPGPGPGPGPGPGPGPGPGPGPGPGPGPKISITEIKGVIVHRIETILF

SEQ ID NO: 122 - protein, 66 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): (GP)10
  PEPTIDE (44)..(47): epsilon K-KKK as a spacer
  SITE (47)..(47): epsilon-K
  PEPTIDE (48)..(66): UBITh3
  SITE (51)..(51): S or T
  SITE (54)..(54): K or R
  SITE (55)..(55): G or T
  SITE (59)..(59): H or T
  SITE (60)..(60): K or R
ISITEIKGVIVHRIETILFKKKKGPGPGPGPGPGPGPGPGPGPKKKKISIXEIXXVIVXXIETILF

SEQ ID NO: 123 - protein, 76 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(53): (GP)15
  PEPTIDE (54)..(57): epsilon K-KKK as a spacer
  SITE (57)..(57): epsilon-K
  PEPTIDE (58)..(76): UBITh3
  SITE (61)..(61): S or T
  SITE (64)..(64): K or R
  SITE (65)..(65): G or T
  SITE (69)..(69): H or T
  SITE (70)..(70): K or R
ISITEIKGVIVHRIETILFKKKKGPGPGPGPGPGPGPGPGPGPGPGPGPGPGPKKKKISIXEIXXVIVXXIETILF

SEQ ID NO: 124 - protein, 96 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(73): (GP)25
  PEPTIDE (74)..(77): epsilon K-KKK as a spacer
  SITE (77)..(77): epsilon-K
  PEPTIDE (78)..(96): UBITh3
  SITE (81)..(81): S or T
  SITE (84)..(84): K or R
  SITE (85)..(85): G or T
  SITE (89)..(89): H or T
  SITE (90)..(90): K or R
ISITEIKGVIVHRIETILFKKKKGPGPGPGPGPGPGPGPGPGPGPGPGPGPGPGPGPGPGPGPGPGPGPGPGPKKKKISIXEIXXVIVXXIETILF

SEQ ID NO: 125 - protein, 60 aa, Artificial Sequence
  Synthetic Peptide
  PEPTIDE (1)..(19): MvF5 Th (UBITh1)
  SITE (20)..(20): epsilon K as a spacer
  PEPTIDE (21)..(40): (GP)10
  SITE (41)..(41): epsilon-K as a spacer
  PEPTIDE (42)..(60): UBITh3
  SITE (45)..(45): S or T
  SITE (48)..(48): K or R
  SITE (49)..(49): G or T
  SITE (53)..(53): H or T
  SITE (54)..(54): K or R
ISITEIKGVIVHRIETILFKGPGPGPGPGPGPGPGPGPGPKISIXEIXXVIVXXIETILF

SEQ ID NO: 126 - protein, 70 aa, Artificial Sequence
  Synthetic Peptide
  PEPTIDE (1)..(19): MvF5 Th (UBITh1)
  SITE (20)..(20): epsilon-K as a spacer
  PEPTIDE (21)..(50): (GP)15
  SITE (51)..(51): epsilon-K as a spacer
  PEPTIDE (52)..(70): UBITh3
  SITE (55)..(55): S or T
  SITE (58)..(58): K or R
  SITE (59)..(59): G or T
  SITE (63)..(63): H or T
  SITE (64)..(64): K or R
ISITEIKGVIVHRIETILFKGPGPGPGPGPGPGPGPGPGPGPGPGPGPGPKISIXEIXXVIVXXIETILF

SEQ ID NO: 127 - protein, 90 aa, Artificial Sequence
  Synthetic Peptide
  PEPTIDE (1)..(19): MvF5 Th (UBITh1)
  SITE (20)..(20): epsilon-K as a spacer
  PEPTIDE (21)..(70): (GP)25
  SITE (71)..(71): epsilon-K as a spacer
  PEPTIDE (72)..(90): UBITh3
  SITE (75)..(75): S or T
  SITE (78)..(78): K or R
  SITE (79)..(79): G or T
  SITE (83)..(83): H or T
  SITE (84)..(84): K or R
ISITEIKGVIVHRIETILFKGPGPGPGPGPGPGPGPGPGPGPGPGPGPGPGPGPGPGPGPGPGPGPGPGPKISIXEIXXVIVXXIETILF

SEQ ID NO: 128 - protein, 43 aa, Artificial Sequence
  Synthetic Peptide
  PEPTIDE (1)..(20): (GR)10
  PEPTIDE (21)..(24): KKK-epsilon K as a spacer
  SITE (24)..(24): epsilon K
  PEPTIDE (25)..(43): MvF5 Th (UBITh1)
GRGRGRGRGRGRGRGRGRGRKKKKISITEIKGVIVHRIETILF

SEQ ID NO: 129 - protein, 53 aa, Artificial Sequence
  Synthetic Peptide
  PEPTIDE (1)..(30): (GR)15
  PEPTIDE (31)..(34): KKK-epsilon K as a spacer
  SITE (34)..(34): epsilon K
  PEPTIDE (35)..(53): MvF5 Th (UBITh1)
GRGRGRGRGRGRGRGRGRGRGRGRGRGRGRKKKKISITEIKGVIVHRIETILF

SEQ ID NO: 130 - protein, 73 aa, Artificial Sequence
  Synthetic Peptide
  PEPTIDE (1)..(50): (GR)25
  PEPTIDE (51)..(54): KKK-epsilon K as a spacer
  SITE (54)..(54): epsilon K
  PEPTIDE (55)..(73): MvF5 Th (UBITh1)
GRGRGRGRGRGRGRGRGRGRGRGRGRGRGRGRGRGRGRGRGRGRGRGRGRKKKKISITEIKGVIVHRIETILF

SEQ ID NO: 131 - protein, 40 aa, Artificial Sequence
  Synthetic Peptide
  PEPTIDE (1)..(20): (GR)10
  SITE (21)..(21): epsilon K
  PEPTIDE (22)..(40): MvF5 Th (UBITh1)
GRGRGRGRGRGRGRGRGRGRKISITEIKGVIVHRIETILF

SEQ ID NO: 132 - protein, 50 aa, Artificial Sequence
  Synthetic Peptide
  PEPTIDE (1)..(30): (GR)15
  SITE (31)..(31): epsilon K as a spacer
  PEPTIDE (32)..(50): MvF5 Th (UBITh1)
GRGRGRGRGRGRGRGRGRGRGRGRGRGRGRKISITEIKGVIVHRIETILF

SEQ ID NO: 133 - protein, 70 aa, Artificial Sequence
  Synthetic Peptide
  PEPTIDE (1)..(50): (GR)25
  SITE (51)..(51): epsilon K as a spacer
  PEPTIDE (52)..(70): MvF5 Th (UBITh1)
GRGRGRGRGRGRGRGRGRGRGRGRGRGRGRGRGRGRGRGRGRGRGRGRGRKISITEIKGVIVHRIETILF

SEQ ID NO: 134 - protein, 43 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): (GR)10
ISITEIKGVIVHRIETILFKKKKGRGRGRGRGRGRGRGRGRGR

SEQ ID NO: 135 - protein, 53 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(53): (GR)15
ISITEIKGVIVHRIETILFKKKKGRGRGRGRGRGRGRGRGRGRGRGRGRGRGR

SEQ ID NO: 136 - protein, 73 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(73): (GR)25
ISITEIKGVIVHRIETILFKKKKGRGRGRGRGRGRGRGRGRGRGRGRGRGRGRGRGRGRGRGRGRGRGRGRGR

SEQ ID NO: 137 - protein, 40 aa, Artificial Sequence
  Synthetic Peptide
  PEPTIDE (1)..(19): MvF5 Th (UBITh1)
  SITE (20)..(20): epsilon-K
  PEPTIDE (21)..(40): (GA)10
ISITEIKGVIVHRIETILFKGRGRGRGRGRGRGRGRGRGR

SEQ ID NO: 138 - protein, 50 aa, Artificial Sequence
  Synthetic Peptide
  PEPTIDE (1)..(19): MvF5 Th (UBITh1)
  SITE (20)..(20): epsilon-K
  PEPTIDE (21)..(50): (GA)15
ISITEIKGVIVHRIETILFKGRGRGRGRGRGRGRGRGRGRGRGRGRGRGR

SEQ ID NO: 139 - protein, 70 aa, Artificial Sequence
  Synthetic Peptide
  PEPTIDE (1)..(19): MvF5 Th (UBITh1)
  SITE (20)..(20): epsilon-K
  PEPTIDE (21)..(70): (GR)25
ISITEIKGVIVHRIETILFKGRGRGRGRGRGRGRGRGRGRGRGRGRGRGRGRGRGRGRGRGRGRGRGRGR

SEQ ID NO: 140 - protein, 66 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): (GR)10
  PEPTIDE (44)..(47): epsilon K-KKK as a spacer
  SITE (47)..(47): epsilon-K
  PEPTIDE (48)..(66): MvF5 Th (UBITh1)
ISITEIKGVIVHRIETILFKKKKGRGRGRGRGRGRGRGRGRGRKKKKISITEIKGVIVHRIETILF

SEQ ID NO: 141 - protein, 76 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(53): (GR)15
  PEPTIDE (54)..(57): epsilon K-KKK as a spacer
  SITE (57)..(57): epsilon-K
  PEPTIDE (58)..(76): MvF5 Th (UBITh1)
ISITEIKGVIVHRIETILFKKKKGRGRGRGRGRGRGRGRGRGRGRGRGRGRGRKKKKISITEIKGVIVHRIETILF

SEQ ID NO: 142 - protein, 96 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(73): (GR)25
  PEPTIDE (74)..(77): epsilon K-KKK as a spacer
  SITE (77)..(77): epsilon-K
  PEPTIDE (78)..(96): MvF5 Th (UBITh1)
ISITEIKGVIVHRIETILFKKKKGRGRGRGRGRGRGRGRGRGRGRGRGRGRGRGRGRGRGRGRGRGRGRGRGRKKKKISITEIKGVIVHRIETILF

SEQ ID NO: 143 - protein, 60 aa, Artificial Sequence
  Synthetic Peptide
  PEPTIDE (1)..(19): MvF5 Th (UBITh1)
  SITE (20)..(20): epsilon-K as a spacer
  PEPTIDE (21)..(40): (GR)10
  SITE (41)..(41): epsilon-K
  PEPTIDE (42)..(60): MvF5 Th (UBITh1)
ISITEIKGVIVHRIETILFKGRGRGRGRGRGRGRGRGRGRKISITEIKGVIVHRIETILF

SEQ ID NO: 144 - protein, 70 aa, Artificial Sequence
  Synthetic Peptide
  PEPTIDE (1)..(19): MvF5 Th (UBITh1)
  SITE (20)..(20): epsilon K as a spacer
  PEPTIDE (21)..(50): (GR)15
  SITE (51)..(51): epsilon K as a spacer
  PEPTIDE (52)..(70): MvF5 Th (UBITh1)
ISITEIKGVIVHRIETILFKGRGRGRGRGRGRGRGRGRGRGRGRGRGRGRKISITEIKGVIVHRIETILF

SEQ ID NO: 145 - protein, 90 aa, Artificial Sequence
  Synthetic Peptide
  PEPTIDE (1)..(19): MvF5 Th (UBITh1)
  SITE (20)..(20): epsilon K as a spacer
  PEPTIDE (21)..(70): (GR)25
  SITE (71)..(71): epsilon K as a spacer
  PEPTIDE (72)..(90): MvF5 Th (UBITh1)
ISITEIKGVIVHRIETILFKGRGRGRGRGRGRGRGRGRGRGRGRGRGRGRGRGRGRGRGRGRGRGRGRGRKISITEIKGVIVHRIETILF

SEQ ID NO: 146 - protein, 66 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(43): (GR)10
  PEPTIDE (44)..(47): epsilon K-KKK as a spacer
  SITE (47)..(47): epsilon-K
  PEPTIDE (48)..(66): MvF5 Th (UBITh1)
ISIXEIXXVIVXXIETILFKKKKGRGRGRGRGRGRGRGRGRGRKKKKISITEIKGVIVHRIETILF

SEQ ID NO: 147 - protein, 76 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(53): (GR)15
  PEPTIDE (54)..(57): epsilon K-KKK as a spacer
  SITE (57)..(57): epsilon-K
  PEPTIDE (58)..(76): MvF5 Th (UBITh1)
ISIXEIXXVIVXXIETILFKKKKGRGRGRGRGRGRGRGRGRGRGRGRGRGRGRKKKKISITEIKGVIVHRIETILF

SEQ ID NO: 148 - protein, 96 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(73): (GR)25
  PEPTIDE (74)..(77): epsilon K-KKK as a spacer
  SITE (77)..(77): epsilon-K
  PEPTIDE (78)..(96): MvF5 Th (UBITh1)
ISIXEIXXVIVXXIETILFKKKKGRGRGRGRGRGRGRGRGRGRGRGRGRGRGRGRGRGRGRGRGRGRGRGRGRKKKKISITEIKGVIVHRIETILF

SEQ ID NO: 149 - protein, 60 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(40): (GR)10
  SITE (41)..(41): epsilon-K as a spacer
  PEPTIDE (42)..(60): MvF5 Th (UBITh1)
ISIXEIXXVIVXXIETILFKGRGRGRGRGRGRGRGRGRGRKISITEIKGVIVHRIETILF

SEQ ID NO: 150 - protein, 70 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(50): (GR)15
  SITE (51)..(51): epsilon-K as a spacer
  PEPTIDE (52)..(70): MvF5 Th (UBITh1)
ISIXEIXXVIVXXIETILFKGRGRGRGRGRGRGRGRGRGRGRGRGRGRGRKISITEIKGVIVHRIETILF

SEQ ID NO: 151 - protein, 90 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(70): (GR)25
  SITE (71)..(71): epsilon-K as a spacer
  PEPTIDE (72)..(90): MvF5 Th (UBITh1)
ISIXEIXXVIVXXIETILFKGRGRGRGRGRGRGRGRGRGRGRGRGRGRGRGRGRGRGRGRGRGRGRGRGRKISITEIKGVIVHRIETILF

SEQ ID NO: 152 - protein, 66 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): (GR)10
  PEPTIDE (44)..(47): epsilon K-KKK as a spacer
  SITE (47)..(47): epsilon-K
  PEPTIDE (48)..(66): UBITh3
  SITE (51)..(51): S or T
  SITE (54)..(54): K or R
  SITE (55)..(55): G or T
  SITE (59)..(59): H or T
  SITE (60)..(60): K or R
ISITEIKGVIVHRIETILFKKKKGRGRGRGRGRGRGRGRGRGRKKKKISIXEIXXVIVXXIETILF

SEQ ID NO: 153 - protein, 76 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(53): (GR)15
  PEPTIDE (54)..(57): epsilon K-KKK as a spacer
  SITE (57)..(57): epsilon-K
  PEPTIDE (58)..(76): UBITh3
  SITE (61)..(61): S or T
  SITE (64)..(64): K or R
  SITE (65)..(65): G or T
  SITE (69)..(69): H or T
  SITE (70)..(70): K or R
ISITEIKGVIVHRIETILFKKKKGRGRGRGRGRGRGRGRGRGRGRGRGRGRGRKKKKISIXEIXXVIVXXIETILF

SEQ ID NO: 154 - protein, 96 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(73): (GR)25
  PEPTIDE (74)..(77): epsilon K-KKK as a spacer
  SITE (77)..(77): epsilon-K
  PEPTIDE (78)..(96): UBITh3
  SITE (81)..(81): S or T
  SITE (84)..(84): K or R
  SITE (85)..(85): G or T
  SITE (89)..(89): H or T
  SITE (90)..(90): K or R
ISITEIKGVIVHRIETILFKKKKGRGRGRGRGRGRGRGRGRGRGRGRGRGRGRGRGRGRGRGRGRGRGRGRGRKKKKISIXEIXXVIVXXIETILF

SEQ ID NO: 155 - protein, 60 aa, Artificial Sequence
  Synthetic Peptide
  PEPTIDE (1)..(19): MvF5 Th (UBITh1)
  SITE (20)..(20): epsilon K as a spacer
  PEPTIDE (21)..(40): (GR)10
  SITE (41)..(41): epsilon-K as a spacer
  PEPTIDE (42)..(60): UBITh3
  SITE (45)..(45): S or T
  SITE (48)..(48): K or R
  SITE (49)..(49): G or T
  SITE (53)..(53): H or T
  SITE (54)..(54): K or R
ISITEIKGVIVHRIETILFKGRGRGRGRGRGRGRGRGRGRKISIXEIXXVIVXXIETILF

SEQ ID NO: 156 - protein, 70 aa, Artificial Sequence
  Synthetic Peptide
  PEPTIDE (1)..(19): MvF5 Th (UBITh1)
  SITE (20)..(20): epsilon-K as a spacer
  PEPTIDE (21)..(50): (GR)15
  SITE (51)..(51): epsilon-K as a spacer
  PEPTIDE (52)..(70): UBITh3
  SITE (55)..(55): S or T
  SITE (58)..(58): K or R
  SITE (59)..(59): G or T
  SITE (63)..(63): H or T
  SITE (64)..(64): K or R
ISITEIKGVIVHRIETILFKGRGRGRGRGRGRGRGRGRGRGRGRGRGRGRKISIXEIXXVIVXXIETILF

SEQ ID NO: 157 - protein, 90 aa, Artificial Sequence
  Synthetic Peptide
  PEPTIDE (1)..(19): MvF5 Th (UBITh1)
  SITE (20)..(20): epsilon-K as a spacer
  PEPTIDE (21)..(70): (GR)25
  SITE (71)..(71): epsilon-K as a spacer
  PEPTIDE (72)..(90): UBITh3
  SITE (75)..(75): S or T
  SITE (78)..(78): K or R
  SITE (79)..(79): G or T
  SITE (83)..(83): H or T
  SITE (84)..(84): K or R
ISITEIKGVIVHRIETILFKGRGRGRGRGRGRGRGRGRGRGRGRGRGRGRGRGRGRGRGRGRGRGRGRGRKISIXEIXXVIVXXIETILF

SEQ ID NO: 158 - protein, 43 aa, Artificial Sequence
  Synthetic Peptide
  PEPTIDE (1)..(20): (PR)10
  PEPTIDE (21)..(24): KKK-epsilon K as a spacer
  SITE (24)..(24): epsilon K
  PEPTIDE (25)..(43): MvF5 Th (UBITh1)
PRPRPRPRPRPRPRPRPRPRKKKKISITEIKGVIVHRIETILF

SEQ ID NO: 159 - protein, 53 aa, Artificial Sequence
  Synthetic Peptide
  PEPTIDE (1)..(30): (PR)15
  PEPTIDE (31)..(34): KKK-epsilon K as a spacer
  SITE (34)..(34): epsilon K
  PEPTIDE (35)..(53): MvF5 Th (UBITh1)
PRPRPRPRPRPRPRPRPRPRPRPRPRPRPRKKKKISITEIKGVIVHRIETILF

SEQ ID NO: 160 - protein, 73 aa, Artificial Sequence
  Synthetic Peptide
  PEPTIDE (1)..(50): (PR)25
  PEPTIDE (51)..(54): KKK-epsilon K as a spacer
  SITE (54)..(54): epsilon K
  PEPTIDE (55)..(73): MvF5 Th (UBITh1)
PRPRPRPRPRPRPRPRPRPRPRPRPRPRPRPRPRPRPRPRPRPRPRPRPRKKKKISITEIKGVIVHRIETILF

SEQ ID NO: 161 - protein, 40 aa, Artificial Sequence
  Synthetic Peptide
  PEPTIDE (1)..(20): (PR)10
  SITE (21)..(21): epsilon K
  PEPTIDE (22)..(40): MvF5 Th (UBITh1)
PRPRPRPRPRPRPRPRPRPRKISITEIKGVIVHRIETILF

SEQ ID NO: 162 - protein, 50 aa, Artificial Sequence
  Synthetic Peptide
  PEPTIDE (1)..(30): (PR)15
  SITE (31)..(31): epsilon K as a spacer
  PEPTIDE (32)..(50): MvF5 Th (UBITh1)
PRPRPRPRPRPRPRPRPRPRPRPRPRPRPRKISITEIKGVIVHRIETILF

SEQ ID NO: 163 - protein, 70 aa, Artificial Sequence
  Synthetic Peptide
  PEPTIDE (1)..(50): (PR)25
  SITE (51)..(51): epsilon K as a spacer
  PEPTIDE (52)..(70): MvF5 Th (UBITh1)
PRPRPRPRPRPRPRPRPRPRPRPRPRPRPRPRPRPRPRPRPRPRPRPRPRKISITEIKGVIVHRIETILF

SEQ ID NO: 164 - protein, 43 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): (PR)10
ISITEIKGVIVHRIETILFKKKKPRPRPRPRPRPRPRPRPRPR

SEQ ID NO: 165 - protein, 53 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(53): (PR)15
ISITEIKGVIVHRIETILFKKKKPRPRPRPRPRPRPRPRPRPRPRPRPRPRPR

SEQ ID NO: 166 - protein, 73 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(73): (PR)25
ISITEIKGVIVHRIETILFKKKKPRPRPRPRPRPRPRPRPRPRPRPRPRPRPRPRPRPRPRPRPRPRPRPRPR

SEQ ID NO: 167 - protein, 40 aa, Artificial Sequence
  Synthetic Peptide
  PEPTIDE (1)..(19): MvF5 Th (UBITh1)
  SITE (20)..(20): epsilon-K
  PEPTIDE (21)..(40): (PR)10
ISITEIKGVIVHRIETILFKPRPRPRPRPRPRPRPRPRPR

SEQ ID NO: 168 - protein, 50 aa, Artificial Sequence
  Synthetic Peptide
  PEPTIDE (1)..(19): MvF5 Th (UBITh1)
  SITE (20)..(20): epsilon-K
  PEPTIDE (21)..(50): (PR)15
ISITEIKGVIVHRIETILFKPRPRPRPRPRPRPRPRPRPRPRPRPRPRPR

SEQ ID NO: 169 - protein, 70 aa, Artificial Sequence
  Synthetic Peptide
  PEPTIDE (1)..(19): MvF5 Th (UBITh1)
  SITE (20)..(20): epsilon-K
  PEPTIDE (21)..(70): (PR)25
ISITEIKGVIVHRIETILFKPRPRPRPRPRPRPRPRPRPRPRPRPRPRPRPRPRPRPRPRPRPRPRPRPR

SEQ ID NO: 170 - protein, 66 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): (PR)10
  PEPTIDE (44)..(47): epsilon K-KKK as a spacer
  SITE (47)..(47): epsilon-K
  PEPTIDE (48)..(66): MvF5 Th (UBITh1)
ISITEIKGVIVHRIETILFKKKKPRPRPRPRPRPRPRPRPRPRKKKKISITEIKGVIVHRIETILF

SEQ ID NO: 171 - protein, 76 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(53): (PR)15
  PEPTIDE (54)..(57): epsilon K-KKK as a spacer
  SITE (57)..(57): epsilon-K
  PEPTIDE (58)..(76): MvF5 Th (UBITh1)
ISITEIKGVIVHRIETILFKKKKPRPRPRPRPRPRPRPRPRPRPRPRPRPRPRKKKKISITEIKGVIVHRIETILF

SEQ ID NO: 172 - protein, 96 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(73): (PR)25
  PEPTIDE (74)..(77): epsilon K-KKK as a spacer
  SITE (77)..(77): epsilon-K
  PEPTIDE (78)..(96): MvF5 Th (UBITh1)
ISITEIKGVIVHRIETILFKKKKPRPRPRPRPRPRPRPRPRPRPRPRPRPRPRPRPRPRPRPRPRPRPRPRPRKKKKISITEIKGVIVHRIETILF

SEQ ID NO: 173 - protein, 60 aa, Artificial Sequence
  Synthetic Peptide
  PEPTIDE (1)..(19): MvF5 Th (UBITh1)
  SITE (20)..(20): epsilon-K as a spacer
  PEPTIDE (21)..(40): (PR)10
  SITE (41)..(41): epsilon-K
  PEPTIDE (42)..(60): MvF5 Th (UBITh1)
ISITEIKGVIVHRIETILFKPRPRPRPRPRPRPRPRPRPRKISITEIKGVIVHRIETILF

SEQ ID NO: 174 - protein, 70 aa, Artificial Sequence
  Synthetic Peptide
  PEPTIDE (1)..(19): MvF5 Th (UBITh1)
  SITE (20)..(20): epsilon K as a spacer
  PEPTIDE (21)..(50): (PR)15
  SITE (51)..(51): epsilon K as a spacer
  PEPTIDE (52)..(70): MvF5 Th (UBITh1)
ISITEIKGVIVHRIETILFKPRPRPRPRPRPRPRPRPRPRPRPRPRPRPRKISITEIKGVIVHRIETILF

SEQ ID NO: 175 - protein, 90 aa, Artificial Sequence
  Synthetic Peptide
  PEPTIDE (1)..(19): MvF5 Th (UBITh1)
  SITE (20)..(20): epsilon K as a spacer
  PEPTIDE (21)..(70): (PR)25
  SITE (71)..(71): epsilon K as a spacer
  PEPTIDE (72)..(90): MvF5 Th (UBITh1)
ISITEIKGVIVHRIETILFKPRPRPRPRPRPRPRPRPRPRPRPRPRPRPRPRPRPRPRPRPRPRPRPRPRKISITEIKGVIVHRIETILF

SEQ ID NO: 176 - protein, 66 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(43): (PR)10
  PEPTIDE (44)..(47): epsilon K-KKK as a spacer
  SITE (47)..(47): epsilon-K
  PEPTIDE (48)..(66): MvF5 Th (UBITh1)
ISIXEIXXVIVXXIETILFKKKKPRPRPRPRPRPRPRPRPRPRKKKKISITEIKGVIVHRIETILF

SEQ ID NO: 177 - protein, 76 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(53): PR)15
  PEPTIDE (54)..(57): epsilon K-KKK as a spacer
  SITE (57)..(57): epsilon-K
  PEPTIDE (58)..(76): MvF5 Th (UBITh1)
ISIXEIXXVIVXXIETILFKKKKPRPRPRPRPRPRPRPRPRPRPRPRPRPRPRKKKKISITEIKGVIVHRIETILF

SEQ ID NO: 178 - protein, 96 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(73): (PR)25
  PEPTIDE (74)..(77): epsilon K-KKK as a spacer
  SITE (77)..(77): epsilon-K
  PEPTIDE (78)..(96): MvF5 Th (UBITh1)
ISIXEIXXVIVXXIETILFKKKKPRPRPRPRPRPRPRPRPRPRPRPRPRPRPRPRPRPRPRPRPRPRPRPRPRKKKKISITEIKGVIVHRIETILF

SEQ ID NO: 179 - protein, 60 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(40): (PR)10
  SITE (41)..(41): epsilon-K as a spacer
  PEPTIDE (42)..(60): MvF5 Th (UBITh1)
ISIXEIXXVIVXXIETILFKPRPRPRPRPRPRPRPRPRPRKISITEIKGVIVHRIETILF

SEQ ID NO: 180 - protein, 70 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(50): (PR)15
  SITE (51)..(51): epsilon-K as a spacer
  PEPTIDE (52)..(70): MvF5 Th (UBITh1)
ISIXEIXXVIVXXIETILFKPRPRPRPRPRPRPRPRPRPRPRPRPRPRPRKISITEIKGVIVHRIETILF

SEQ ID NO: 181 - protein, 90 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(70): (PR)25
  SITE (71)..(71): epsilon-K as a spacer
  PEPTIDE (72)..(90): MvF5 Th (UBITh1)
ISIXEIXXVIVXXIETILFKPRPRPRPRPRPRPRPRPRPRPRPRPRPRPRPRPRPRPRPRPRPRPRPRPRKISITEIKGVIVHRIETILF

SEQ ID NO: 182 - protein, 66 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): (PR)10
  PEPTIDE (44)..(47): epsilon K-KKK as a spacer
  SITE (47)..(47): epsilon-K
  PEPTIDE (48)..(66): UBITh3
  SITE (51)..(51): S or T
  SITE (54)..(54): K or R
  SITE (55)..(55): G or T
  SITE (59)..(59): H or T
  SITE (60)..(60): K or R
ISITEIKGVIVHRIETILFKKKKPRPRPRPRPRPRPRPRPRPRKKKKISIXEIXXVIVXXIETILF

SEQ ID NO: 183 - protein, 76 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(53): (PR)15
  PEPTIDE (54)..(57): epsilon K-KKK as a spacer
  SITE (57)..(57): epsilon-K
  PEPTIDE (58)..(76): UBITh3
  SITE (61)..(61): S or T
  SITE (64)..(64): K or R
  SITE (65)..(65): G or T
  SITE (69)..(69): H or T
  SITE (70)..(70): K or R
ISITEIKGVIVHRIETILFKKKKPRPRPRPRPRPRPRPRPRPRPRPRPRPRPRKKKKISIXEIXXVIVXXIETILF

SEQ ID NO: 184 - protein, 96 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(73): (PR)25
  PEPTIDE (74)..(77): epsilon K-KKK as a spacer
  SITE (77)..(77): epsilon-K
  PEPTIDE (78)..(96): UBITh3
  SITE (81)..(81): S or T
  SITE (84)..(84): K or R
  SITE (85)..(85): G or T
  SITE (89)..(89): H or T
  SITE (90)..(90): K or R
ISITEIKGVIVHRIETILFKKKKPRPRPRPRPRPRPRPRPRPRPRPRPRPRPRPRPRPRPRPRPRPRPRPRPRKKKKISIXEIXXVIVXXIETILF

SEQ ID NO: 185 - protein, 60 aa, Artificial Sequence
  Synthetic Peptide
  PEPTIDE (1)..(19): MvF5 Th (UBITh1)
  SITE (20)..(20): epsilon K as a spacer
  PEPTIDE (21)..(40): (PR)10
  SITE (41)..(41): epsilon-K as a spacer
  PEPTIDE (42)..(60): UBITh3
  SITE (45)..(45): S or T
  SITE (48)..(48): K or R
  SITE (49)..(49): G or T
  SITE (53)..(53): H or T
  SITE (54)..(54): K or R
ISITEIKGVIVHRIETILFKPRPRPRPRPRPRPRPRPRPRKISIXEIXXVIVXXIETILF

SEQ ID NO: 186 - protein, 70 aa, Artificial Sequence
  Synthetic Peptide
  PEPTIDE (1)..(19): MvF5 Th (UBITh1)
  SITE (20)..(20): epsilon-K as a spacer
  PEPTIDE (21)..(50): (PR)15
  SITE (51)..(51): epsilon-K as a spacer
  PEPTIDE (52)..(70): UBITh3
  SITE (55)..(55): S or T
  SITE (58)..(58): K or R
  SITE (59)..(59): G or T
  SITE (63)..(63): H or T
  SITE (64)..(64): K or R
ISITEIKGVIVHRIETILFKPRPRPRPRPRPRPRPRPRPRPRPRPRPRPRKISIXEIXXVIVXXIETILF

SEQ ID NO: 187 - protein, 90 aa, Artificial Sequence
  Synthetic Peptide
  PEPTIDE (1)..(19): MvF5 Th (UBITh1)
  SITE (20)..(20): epsilon-K as a spacer
  PEPTIDE (21)..(70): (PR)25
  SITE (71)..(71): epsilon-K as a spacer
  PEPTIDE (72)..(90): UBITh3
  SITE (75)..(75): S or T
  SITE (78)..(78): K or R
  SITE (79)..(79): G or T
  SITE (83)..(83): H or T
  SITE (84)..(84): K or R
ISITEIKGVIVHRIETILFKPRPRPRPRPRPRPRPRPRPRPRPRPRPRPRPRPRPRPRPRPRPRPRPRPRKISIXEIXXVIVXXIETILF

SEQ ID NO: 188 - protein, 43 aa, Artificial Sequence
  Synthetic Peptide
  PEPTIDE (1)..(20): (PA)10
  PEPTIDE (21)..(24): KKK-epsilon K as a spacer
  SITE (24)..(24): epsilon K
  PEPTIDE (25)..(43): MvF5 Th (UBITh1)
PAPAPAPAPAPAPAPAPAPAKKKKISITEIKGVIVHRIETILF

SEQ ID NO: 189 - protein, 53 aa, Artificial Sequence
  Synthetic Peptide
  PEPTIDE (1)..(30): (PA)15
  PEPTIDE (31)..(34): KKK-epsilon K as a spacer
  SITE (34)..(34): epsilon K
  PEPTIDE (35)..(53): MvF5 Th (UBITh1)
PAPAPAPAPAPAPAPAPAPAPAPAPAPAPAKKKKISITEIKGVIVHRIETILF

SEQ ID NO: 190 - protein, 73 aa, Artificial Sequence
  Synthetic Peptide
  PEPTIDE (1)..(50): (PA)25
  PEPTIDE (51)..(54): KKK-epsilon K as a spacer
  SITE (54)..(54): epsilon K
  PEPTIDE (55)..(73): MvF5 Th (UBITh1)
PAPAPAPAPAPAPAPAPAPAPAPAPAPAPAPAPAPAPAPAPAPAPAPAPAKKKKISITEIKGVIVHRIETILF

SEQ ID NO: 191 - protein, 40 aa, Artificial Sequence
  Synthetic Peptide
  PEPTIDE (1)..(20): (PA)10
  SITE (21)..(21): epsilon K
  PEPTIDE (22)..(40): MvF5 Th (UBITh1)
PAPAPAPAPAPAPAPAPAPAKISITEIKGVIVHRIETILF

SEQ ID NO: 192 - protein, 50 aa, Artificial Sequence
  Synthetic Peptide
  PEPTIDE (1)..(30): (PA)15
  SITE (31)..(31): epsilon K as a spacer
  PEPTIDE (32)..(50): MvF5 Th (UBITh1)
PAPAPAPAPAPAPAPAPAPAPAPAPAPAPAKISITEIKGVIVHRIETILF

SEQ ID NO: 193 - protein, 70 aa, Artificial Sequence
  Synthetic Peptide
  PEPTIDE (1)..(50): (PA)25
  SITE (51)..(51): epsilon K as a spacer
  PEPTIDE (52)..(70): MvF5 Th (UBITh1)
PAPAPAPAPAPAPAPAPAPAPAPAPAPAPAPAPAPAPAPAPAPAPAPAPAKISITEIKGVIVHRIETILF

SEQ ID NO: 194 - protein, 43 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): (PA)10
ISITEIKGVIVHRIETILFKKKKPAPAPAPAPAPAPAPAPAPA

SEQ ID NO: 195 - protein, 53 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(53): (PA)15
ISITEIKGVIVHRIETILFKKKKPAPAPAPAPAPAPAPAPAPAPAPAPAPAPA

SEQ ID NO: 196 - protein, 73 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(73): (PA)25
ISITEIKGVIVHRIETILFKKKKPAPAPAPAPAPAPAPAPAPAPAPAPAPAPAPAPAPAPAPAPAPAPAPAPA

SEQ ID NO: 197 - protein, 40 aa, Artificial Sequence
  Synthetic Peptide
  PEPTIDE (1)..(19): MvF5 Th (UBITh1)
  SITE (20)..(20): epsilon-K
  PEPTIDE (21)..(40): (PA)10
ISITEIKGVIVHRIETILFKPAPAPAPAPAPAPAPAPAPA

SEQ ID NO: 198 - protein, 50 aa, Artificial Sequence
  Synthetic Peptide
  PEPTIDE (1)..(19): MvF5 Th (UBITh1)
  SITE (20)..(20): epsilon-K
  PEPTIDE (21)..(50): (PA)15
ISITEIKGVIVHRIETILFKPAPAPAPAPAPAPAPAPAPAPAPAPAPAPA

SEQ ID NO: 199 - protein, 70 aa, Artificial Sequence
  Synthetic Peptide
  PEPTIDE (1)..(19): MvF5 Th (UBITh1)
  SITE (20)..(20): epsilon-K
  PEPTIDE (21)..(70): (PA)25
ISITEIKGVIVHRIETILFKPAPAPAPAPAPAPAPAPAPAPAPAPAPAPAPAPAPAPAPAPAPAPAPAPA

SEQ ID NO: 200 - protein, 66 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): (PA)10
  PEPTIDE (44)..(47): epsilon K-KKK as a spacer
  SITE (47)..(47): epsilon-K
  PEPTIDE (48)..(66): MvF5 Th (UBITh1)
ISITEIKGVIVHRIETILFKKKKPAPAPAPAPAPAPAPAPAPAKKKKISITEIKGVIVHRIETILF

SEQ ID NO: 201 - protein, 76 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(53): (PA)15
  PEPTIDE (54)..(57): epsilon K-KKK as a spacer
  SITE (57)..(57): epsilon-K
  PEPTIDE (58)..(76): MvF5 Th (UBITh1)
ISITEIKGVIVHRIETILFKKKKPAPAPAPAPAPAPAPAPAPAPAPAPAPAPAKKKKISITEIKGVIVHRIETILF

SEQ ID NO: 202 - protein, 96 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(73): (PA)25
  PEPTIDE (74)..(77): epsilon K-KKK as a spacer
  SITE (77)..(77): epsilon-K
  PEPTIDE (78)..(96): MvF5 Th (UBITh1)
ISITEIKGVIVHRIETILFKKKKPAPAPAPAPAPAPAPAPAPAPAPAPAPAPAPAPAPAPAPAPAPAPAPAPAKKKKISITEIKGVIVHRIETILF

SEQ ID NO: 203 - protein, 60 aa, Artificial Sequence
  Synthetic Peptide
  PEPTIDE (1)..(19): MvF5 Th (UBITh1)
  SITE (20)..(20): epsilon-K as a spacer
  PEPTIDE (21)..(40): (PA)10
  SITE (41)..(41): epsilon-K
  PEPTIDE (42)..(60): MvF5 Th (UBITh1)
ISITEIKGVIVHRIETILFKPAPAPAPAPAPAPAPAPAPAKISITEIKGVIVHRIETILF

SEQ ID NO: 204 - protein, 70 aa, Artificial Sequence
  Synthetic Peptide
  PEPTIDE (1)..(19): MvF5 Th (UBITh1)
  SITE (20)..(20): epsilon K as a spacer
  PEPTIDE (21)..(50): (PA)15
  SITE (51)..(51): epsilon K as a spacer
  PEPTIDE (52)..(70): MvF5 Th (UBITh1)
ISITEIKGVIVHRIETILFKPAPAPAPAPAPAPAPAPAPAPAPAPAPAPAKISITEIKGVIVHRIETILF

SEQ ID NO: 205 - protein, 90 aa, Artificial Sequence
  Synthetic Peptide
  PEPTIDE (1)..(19): MvF5 Th (UBITh1)
  SITE (20)..(20): epsilon K as a spacer
  PEPTIDE (21)..(70): (PA)25
  SITE (71)..(71): epsilon K as a spacer
  PEPTIDE (72)..(90): MvF5 Th (UBITh1)
ISITEIKGVIVHRIETILFKPAPAPAPAPAPAPAPAPAPAPAPAPAPAPAPAPAPAPAPAPAPAPAPAPAKISITEIKGVIVHRIETILF

SEQ ID NO: 206 - protein, 66 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(43): (PA)10
  PEPTIDE (44)..(47): epsilon K-KKK as a spacer
  SITE (47)..(47): epsilon-K
  PEPTIDE (48)..(66): MvF5 Th (UBITh1)
ISIXEIXXVIVXXIETILFKKKKPAPAPAPAPAPAPAPAPAPAKKKKISITEIKGVIVHRIETILF

SEQ ID NO: 207 - protein, 76 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(53): (PA)15
  PEPTIDE (54)..(57): epsilon K-KKK as a spacer
  SITE (57)..(57): epsilon-K
  PEPTIDE (58)..(76): MvF5 Th (UBITh1)
ISIXEIXXVIVXXIETILFKKKKPAPAPAPAPAPAPAPAPAPAPAPAPAPAPAKKKKISITEIKGVIVHRIETILF

SEQ ID NO: 208 - protein, 96 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(73): (PA)25
  PEPTIDE (74)..(77): epsilon K-KKK as a spacer
  SITE (77)..(77): epsilon-K
  PEPTIDE (78)..(96): MvF5 Th (UBITh1)
ISIXEIXXVIVXXIETILFKKKKPAPAPAPAPAPAPAPAPAPAPAPAPAPAPAPAPAPAPAPAPAPAPAPAPAKKKKISITEIKGVIVHRIETILF

SEQ ID NO: 209 - protein, 60 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(40): (PA)10
  SITE (41)..(41): epsilon-K as a spacer
  PEPTIDE (42)..(60): MvF5 Th (UBITh1)
ISIXEIXXVIVXXIETILFKPAPAPAPAPAPAPAPAPAPAKISITEIKGVIVHRIETILF

SEQ ID NO: 210 - protein, 70 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(50): (PA)15
  SITE (51)..(51): epsilon-K as a spacer
  PEPTIDE (52)..(70): MvF5 Th (UBITh1)
ISIXEIXXVIVXXIETILFKPAPAPAPAPAPAPAPAPAPAPAPAPAPAPAKISITEIKGVIVHRIETILF

SEQ ID NO: 211 - protein, 90 aa, Artificial Sequence
  Synthetic Peptide
  PEPTIDE (1)..(19): UBITh3
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(70): (PA)25
  SITE (71)..(71): epsilon-K as a spacer
  PEPTIDE (72)..(90): MvF5 Th (UBITh1)
ISIXEIXXVIVXXIETILFKPAPAPAPAPAPAPAPAPAPAPAPAPAPAPAPAPAPAPAPAPAPAPAPAPAKISITEIKGVIVHRIETILF

SEQ ID NO: 212 - protein, 66 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): (PA)10
  PEPTIDE (44)..(47): epsilon K-KKK as a spacer
  SITE (47)..(47): epsilon-K
  PEPTIDE (48)..(66): UBITh3
  SITE (51)..(51): S or T
  SITE (54)..(54): K or R
  SITE (55)..(55): G or T
  SITE (59)..(59): H or T
  SITE (60)..(60): K or R
ISITEIKGVIVHRIETILFKKKKPAPAPAPAPAPAPAPAPAPAKKKKISIXEIXXVIVXXIETILF

SEQ ID NO: 213 - protein, 76 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(53): (PA)15
  PEPTIDE (54)..(57): epsilon K-KKK as a spacer
  SITE (57)..(57): epsilon-K
  PEPTIDE (58)..(76): UBITh3
  SITE (61)..(61): S or T
  SITE (64)..(64): K or R
  SITE (65)..(65): G or T
  SITE (69)..(69): H or T
  SITE (70)..(70): K or R
ISITEIKGVIVHRIETILFKKKKPAPAPAPAPAPAPAPAPAPAPAPAPAPAPAKKKKISIXEIXXVIVXXIETILF

SEQ ID NO: 214 - protein, 96 aa, Artificial Sequence
  Synthetic Peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(73): (PA)25
  PEPTIDE (74)..(77): epsilon K-KKK as a spacer
  SITE (77)..(77): epsilon-K
  PEPTIDE (78)..(96): UBITh3
  SITE (81)..(81): S or T
  SITE (84)..(84): K or R
  SITE (85)..(85): G or T
  SITE (89)..(89): H or T
  SITE (90)..(90): K or R
ISITEIKGVIVHRIETILFKKKKPAPAPAPAPAPAPAPAPAPAPAPAPAPAPAPAPAPAPAPAPAPAPAPAPAKKKKISIXEIXXVIVXXIETILF

SEQ ID NO: 215 - protein, 60 aa, Artificial Sequence
  Synthetic Peptide
  PEPTIDE (1)..(19): MvF5 Th (UBITh1)
  SITE (20)..(20): epsilon K as a spacer
  PEPTIDE (21)..(40): (PA)10
  SITE (41)..(41): epsilon-K as a spacer
  PEPTIDE (42)..(60): UBITh3
  SITE (45)..(45): S or T
  SITE (48)..(48): K or R
  SITE (49)..(49): G or T
  SITE (53)..(53): H or T
  SITE (54)..(54): K or R
ISITEIKGVIVHRIETILFKPAPAPAPAPAPAPAPAPAPAKISIXEIXXVIVXXIETILF

SEQ ID NO: 216 - protein, 70 aa, Artificial Sequence
  Synthetic Peptide
  PEPTIDE (1)..(19): MvF5 Th (UBITh1)
  SITE (20)..(20): epsilon-K as a spacer
  PEPTIDE (21)..(50): (PA)15
  SITE (51)..(51): epsilon-K as a spacer
  PEPTIDE (52)..(70): UBITh3
  SITE (55)..(55): S or T
  SITE (58)..(58): K or R
  SITE (59)..(59): G or T
  SITE (63)..(63): H or T
  SITE (64)..(64): K or R
ISITEIKGVIVHRIETILFKPAPAPAPAPAPAPAPAPAPAPAPAPAPAPAKISIXEIXXVIVXXIETILF

SEQ ID NO: 217 - protein, 90 aa, Artificial Sequence
  Synthetic Peptide
  PEPTIDE (1)..(19): MvF5 Th (UBITh1)
  SITE (20)..(20): epsilon-K as a spacer
  PEPTIDE (21)..(70): (PA)25
  SITE (71)..(71): epsilon-K as a spacer
  PEPTIDE (72)..(90): UBITh3
  SITE (75)..(75): S or T
  SITE (78)..(78): K or R
  SITE (79)..(79): G or T
  SITE (83)..(83): H or T
  SITE (84)..(84): K or R
ISITEIKGVIVHRIETILFKPAPAPAPAPAPAPAPAPAPAPAPAPAPAPAPAPAPAPAPAPAPAPAPAPAKISIXEIXXVIVXXIETILF

SEQ ID NO: 218 - protein, 43 aa, Artificial Sequence; identical to SEQ ID NO: 188
  Synthetic Peptide
  PEPTIDE (1)..(20): (PA)10
  SITE (21)..(21): epsilon K
  PEPTIDE (21)..(24): KKK-epsilon K as a spacer
  PEPTIDE (25)..(43): MvF5 Th (UBITh1)

SEQ ID NO: 219 - protein, 53 aa, Artificial Sequence; identical to SEQ ID NO: 189
  Synthetic Peptide
  PEPTIDE (1)..(30): (PA)15
  SITE (31)..(31): epsilon K
  PEPTIDE (31)..(34): KKK-epsilon K as a spacer
  PEPTIDE (35)..(53): MvF5 Th (UBITh1)

SEQ ID NO: 220 - protein, 6 aa, Artificial Sequence
  Synthetic Peptide
  PEPTIDE (1)..(6): Flexible Hinge Spacer
PPXPXP

SEQ ID NO: 221 - protein, 4 aa, Artificial Sequence
  Synthetic Peptide
  SITE (1)..(1): epsilon-K
  PEPTIDE (1)..(4): epsilon-K-KKK as a spacer
KKKK

SEQ ID NO: 222 - protein, 4 aa, Artificial Sequence
  Synthetic Peptide
  PEPTIDE (1)..(4): epsilon-K-KKK as a spacer
  SITE (4)..(4): epsilon-K
KKKK

SEQ ID NO: 223 - DNA, 32 bp, Artificial sequence
  synthetic oligonucleotide
  misc_feature (1)..(32): CpG1
tcgtcgttttgtcgttttgtcgttttgtcgtt

SEQ ID NO: 224 - DNA, 24 bp, Artificial sequence
  synthetic oligonucleotide
  misc_feature (1)..(24): CpG2
tcgtcgttttgtcgttttgtcgtt

SEQ ID NO: 225 - RNA, 24 nt, Homo sapiens
  Intron (1)..(24): C9ORF72 RNA Sense
ggggccggggccggggccggggcc

SEQ ID NO: 226 - protein, 7 aa, Homo sapiens
  PEPTIDE (1)..(7): Poly-GR
GRGRGRG

SEQ ID NO: 227 - protein, 7 aa, Homo sapiens
  PEPTIDE (1)..(7): Poly-GP
GPGPGPG

SEQ ID NO: 228 - protein, 8 aa, Homo sapiens
  PEPTIDE (1)..(8): Poly-GA
GAGAGAGA

SEQ ID NO: 229 - RNA, 24 nt, Homo sapiens
  Intron (1)..(24): C9ORF72 RNA Anti-Sense
ccccggccccggccccggccccgg

SEQ ID NO: 230 - protein, 8 aa, Homo sapiens
  PEPTIDE (1)..(8): Poly-GP
PGPGPGPG

SEQ ID NO: 231 - protein, 7 aa, Homo sapiens
  PEPTIDE (1)..(7): Poly-PR
PRPRPRP

SEQ ID NO: 232 - protein, 7 aa, Homo sapiens
  PEPTIDE (1)..(7): Poly-PA
APAPAPA